(19) United States
(12) Patent Application Publication
(10) Pub. No.: US 2026/0356799 A1
(43) Pub. Date: Nov. 12, 2026

(54) Process and Intermediates for Preparing Ibuzatrelvir

(71) Applicant: Pfizer Inc., New York, NY (US)

(72) Inventors: Christophe Philippe Allais, Ledyard, CT (US); Stéphane Caron, Stonington, CT (US); María González Esguevillas, Groton, CT (US); Samir Kulkarni, East Lyme, CT (US); Philipp Roosen, East Lyme, CT (US)

(73) Assignee: Pfizer Inc., New York, NY (US)

(21) Appl. No.: 19/491,109

(22) PCT Filed: Jun. 7, 2024
(86) PCT No.: PCT/IB2024/055596

§ 371 Date: Dec. 8, 2025

Related U.S. Application Data
(60) Provisional application No. 63/507,347, filed on Jun. 9, 2023.
(60) Provisional application No. 63/571,500, filed on Mar. 29, 2024.
(60) Provisional application No. 63/646,170, filed on May 13, 2024.

(51) Int. Cl. C07D 207/26; C07D 207/16

(52) U.S. Cl. CPC C07D 207/26; C07D 207/16

(57) ABSTRACT

The present disclosure is directed to processes for preparing N-(Methoxycarbonyl)-3-methyl-L-valyl-(4R)-N-{(1S)-1-cyano-2-[(3S)-2-oxopyrrolidin-3-yl]ethyl}-4-(trifluoromethyl)-L-prolinanmide, I, and forms thereof such as the Form I polymorph thereof, a cyclopentyl methyl ether solvate thereof, the isopropyl acetate solvate thereof, the ethyl acetate solvate thereof and a mixture of the Form 1 polymorph thereof and microcrystalline cellulose (I) and to compositions comprising the solid forms and to intermediates used to prepare Compound I.

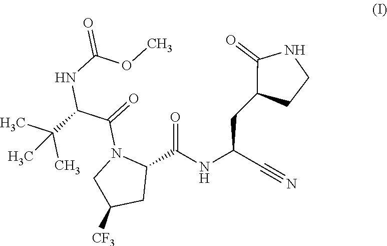 [SMILES: COC(=O)N[C@H](C(=O)N1C[C@H](C(F)(F)F)C[C@H]1C(=O)N[C@H](C#N)C[C@@H]1CCNC1=O)C(C)(C)C]

The present invention is directed to intermediates and an efficient process for preparing Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidin-1-yl]-3,3-dimethyl-1-oxobutan-2-yl}carbamate, solvates thereof and intermediates useful in the preparation of those compounds. Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidin-1-yl]-3,3-dimethyl-1-oxobutan-2-yl}carbamate (also referred hereafter as “Compound I”) is an antiviral compound with potent inhibitory activity against coronavirus 3CL proteases and is an active ingredient under investigation as a potential therapy for the treatment of SARS-CoV-2 (COVID-19) infections. Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate and processes for its preparation have been disclosed in PCT International Patent Application WO 2021/250648, US Patent Application Publication 2022/0062232 A1 and U.S. Pat. Nos. 11,452,711, 11,351,149 and 11,541,034 and U.S. Provisional Patent Application No. 63/507,347. The contents of each of the foregoing references are incorporated by reference in their entirety.

### SUMMARY OF THE INVENTION

The present invention provides intermediates and synthetic processes for preparing Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidin-1-yl]-3,3-dimethyl-1-oxobutan-2-yl}carbamate, solvates thereof and intermediates used in their preparation, which is depicted in Reaction Scheme 1, and which contains several process modifications compared to previously disclosed processes. The product of Steps 1 and 2 in Reaction Scheme 1 is potassium (2S,4R)-1-{(2S)-2-[(methoxycarbonyl)amino]-3,3-dimethylbutanoyl}-4-(trifluoromethyl)pyrrolidine-2-carboxylate (Compound 6). Compound 6 is a new intermediate that has been prepared by a highly diastereoselective process starting from tert-Butyl (2R,4R)-1-{(2S)-2-[(methoxycarbonyl) amino]-3,3-dimethylbutanoyl}-4-(trifluoromethyl)pyrrolidine-2-carboxylate (Compound 4) and proceeding through non-isolated intermediate tert-Butyl (2S,4R)-1-{(2S)-2-[(methoxycarbonyl)amino]-3,3-dimethylbutanoyl}-4-(trifluoromethyl) pyrrolidine-2-carboxylate (Compound 5). Compound 4 is prepared as described hereinbelow from a coupling reaction of tert-Butyl (2R,4R)-4-(trifluoromethyl)pyrrolidine-2-carboxylate hydrochloride (1:1) (Compound 3) with commercially available (2S)-2-[(Methoxycarbonyl)amino]-3,3-dimethylbutanoic acid (Compound 2). Compound 3 is prepared starting from commercially available Di-tert-butyl (2R)-4-oxopyrrolidine-1,2-dicarboxylate (Compound 15) and proceeding through intermediate Compounds 16-18 (Di-tert-butyl (2R)-4-hydroxy-4-(trifluoromethyl)pyrrolidine-1,2-dicarboxylate, Di-tert-butyl (2R)-4-(trifluoromethyl)-2,5-dihydro-1H-pyrrole-1,2-dicarboxylate and Di-tert-butyl (2R,4R)-4-(trifluoromethyl)pyrrolidine-1,2-dicarboxylate, respectively) as described hereinbelow. Compound 6 is then coupled with (2S)-2-Amino-3-[(3S)-2-oxopyrrolidin-3-yl]propanamide hydrochloride (1:1) (Compound 7) to provide methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate (Compound 8). Compound 8 is typically not isolated as a solid and a solution of Compound 8 is carried forward and used in the dehydration step. Compound 8 is dehydrated to form Compound I which can be isolated as a crystalline solvate such as an ethyl acetate (EtOAc), isopropyl acetate (IPAc) or cyclopentyl methyl ether (CPME) solvate. In the Reaction Scheme 1 Compound 6 is a potassium salt and Compound 7 is a hydrochloride salt. It is to be understood that in certain embodiments of the invention an alternative salt other than the Compound 6 potassium salt (such as Compound 6a wherein M⁺ is Na⁺ or Li⁺) or the free acid form of Compound 6 which is (2S,4R)-1-{(2S)-2-[(methoxycarbonyl)amino]-3,3-dimethylbutanoyl}-4-(trifluoromethyl)pyrrolidine-2-carboxylic acid (Compound 6′) can be employed in a similar manner. Likewise the free base form of Compound 7 which is (2S)-2-Amino-3-[(3S)-2-oxopyrrolidin-3-yl]propanamide (Compound 7′) can also be employed in a similar manner.

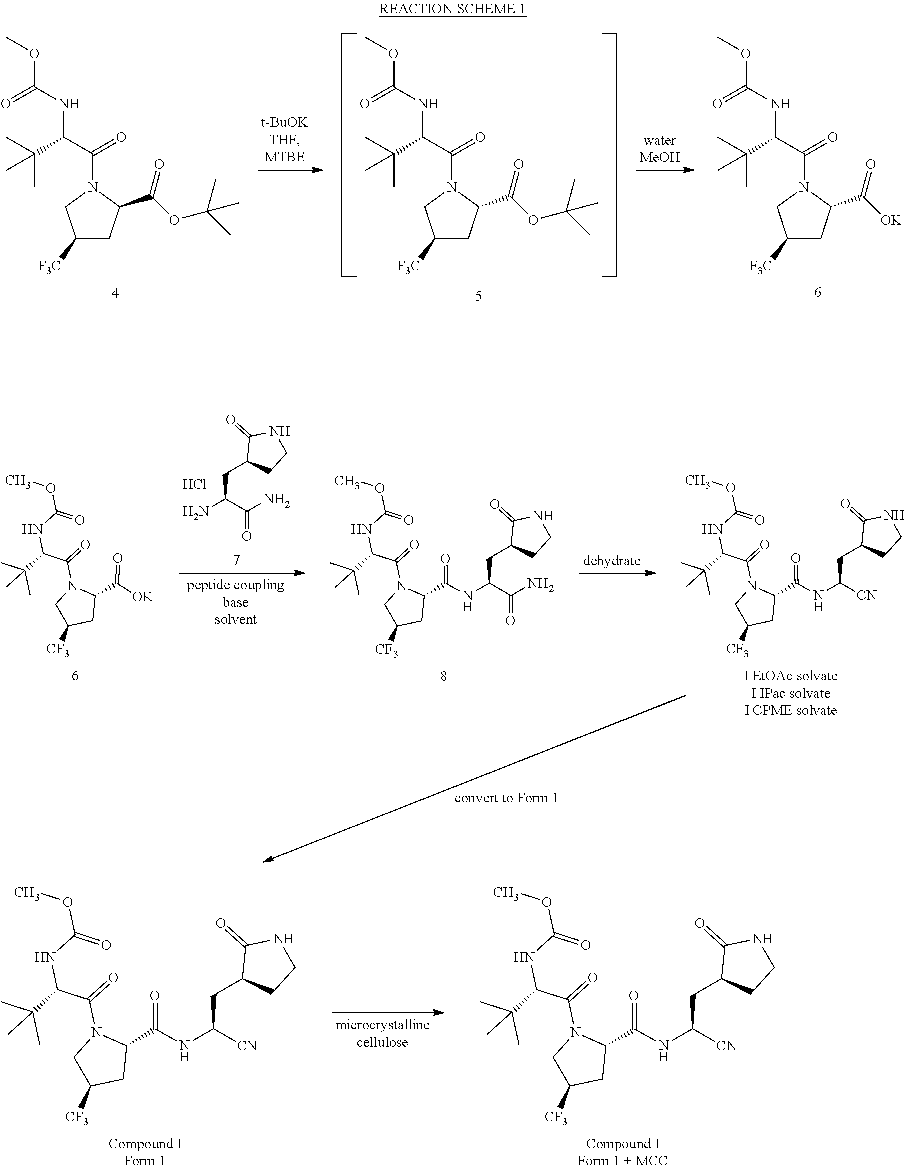

After dehydration of Compound 8 isolation of Compound I in the form of its solvate, such as the ethyl acetate, isopropyl acetate or cyclopentyl methyl ether solvate, is advantageous as it provides a purge point for impurities which allows generation of Compound I Form 1 in high purity. The Compound I solvate is then converted to Compound I Form 1 polymorph. The Compound I Form 1 can then be dissolved and crystallized in the presence of microcrystalline cellulose (MCC) to arrive at Compound I Form 1+MCC in which the Compound I Form 1 has good particle size distribution control. The Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate molecule has a propensity to form solvates with most common solvents and the final form (Compound I Form 1 polymorph) can be achieved by desolvation of solvate. The resulting Compound I solvates are unbound solvates which have tendency to lose solvent even at ambient temperature, which creates additional safety risk in handling the solids. The Compound I solvate step (conversion of Compound 8 to a Compound I solvate) wherein Compound I is isolated as a solvate such as Compound I ethyl acetate solvate, Compound I isopropyl acetate solvate or Compound I cyclopentyl methyl ether solvate has been found to be critical to purge impurities and control particle size of Compound I, Form 1 generated therefrom. Due to the handling and isolation challenges of the solvates, the process to convert the Compound I solvates to Compound I Form 1 was designed without any seeded crystallization, which resulted in the final Compound I Form 1 isolation with no particle size control. The process yielded wide particles size distributions with notable downstream processing challenges for preparing an appropriate drug product. There are various routes of co-processing an active pharmaceutical ingredient and an additive. In one approach, an active pharmaceutical ingredient (API) and non-active component can be crystallized and/or precipitated in solvent-based processes and combined by various mechanisms such as agglomeration, hetero-nucleation, surface coating, and dispersion of API. In the case of Methyl {(2S)-1-[(2S,4R)-2-({(1 S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate, Compound I, it has been found that the additive microcrystalline cellulose can be used as a template to induce nucleation at a given concentration to crystallize the Methyl {(2S)-1-[(2S,4R)-2-({(1S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate in the desired polymorphic form (Form 1) and with desired particle size distributions. It was also found that microcrystalline cellulose-SiO₂ (MCC-SiO₂) can be successfully employed to provide Compound I, Form 1 with the desired particle size distribution whereas other additives such as Di-calcium Phosphate or silicon dioxide (SiO₂) provided little or no control over the Compound I Form 1 particle size distribution. Additional solid form characterization data for several starting materials and intermediates used in the process are also provided.

Reaction Scheme 2 depicts the preparation of Compound 6a starting from Compound 3.

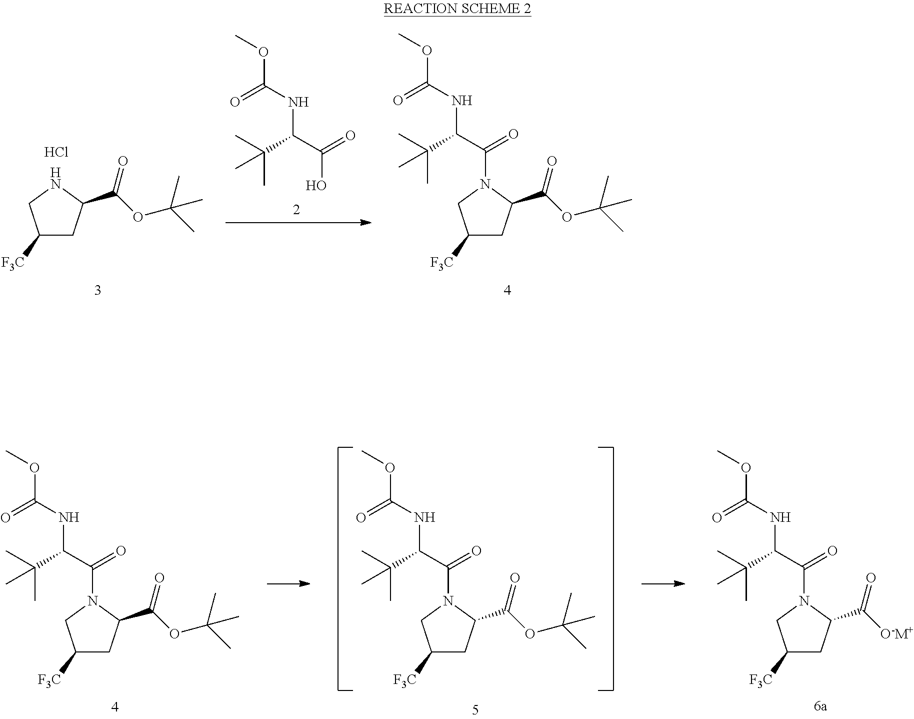

Reaction Scheme 2 depicts preparation of Compound 6a wherein M⁺ is a monovalent cation such as K⁺, Na⁺ or Li⁺. Compound 3 is coupled with Compound 2 under peptide coupling conditions in the presence of a base to provide Compound 4. Compound 4 is then treated with a base which results in formation of Compound 5 in situ which then further reacts to provide Compound 6a.

For the transformation of Compound 4 to Compound 6a various bases and solvents were tried. Use of organic amine bases such as triethylamine, N-methyl imidazole, 1,1,3,3-tetramethylguanidine, triethylenediamine (DABCO) and 1,8-diazabicyclo(5.4.0)undec-7-ene and inorganic bases potassium carbonate (K₂CO₃) led to no formation of Compound 6a from Compound 4. Use of Lithium tert-butoxide resulted in epimerization and trace hydrolysis while sodium tert-butoxide and sodium tert-amylate were found to work but the reaction mixtures tended to form gels. With use of potassium hexamethyldisilazide epimerization at the tert-leucine tert-butyl bearing carbon was observed. With use of potassium methoxide the reaction proceeds but with poor diasteroselectivity and with potassium hydroxide the reaction was slow and proceeded with poor diasteroselectivity. Advantageously, the reaction was found to proceed well and with high diastereoselectivity when potassium tert-butoxide or potassium tert-amylate are used as the base. The reaction with tert-butoxide or potassium tert-amylate works well in solvents including MTBE, THF, IPA, tert-amyl alcohol, acetonitrile, tert-butanol and 2-MeTHF. These reaction conditions are advantageous for manufacturing as the desired product Compound 6a wherein M⁺ is K⁺ can be precipitated from solution and directly isolated.

### BRIEF DESCRIPTION OF THE DRAWINGS

FIG. 1: PXRD Pattern of N-(Methoxycarbonyl)-3-methyl-L-valyl-(4R)-N-{(1S)-1-cyano-2-[(3S)-2-oxopyrrolidin-3-yl]ethyl}-4-(trifluoromethyl)-L-prolinamide, Form 1.

FIG. 2: PXRD Pattern of N-(Methoxycarbonyl)-3-methyl-L-valyl-(4R)-N-{(1S)-1-cyano-2-[(3S)-2-oxopyrrolidin-3-yl]ethyl}-4-(trifluoromethyl)-L-prolinamide cyclopentyl methyl ether (CPME) solvate.

FIG. 3: PXRD Pattern of N-(Methoxycarbonyl)-3-methyl-L-valyl-(4R)-N-{(1S)-1-cyano-2-[(3S)-2-oxopyrrolidin-3-yl]ethyl}-4-(trifluoromethyl)-L-prolinamide isopropyl acetate (IPAc) solvate.

FIG. 4: PXRD Pattern of N-(Methoxycarbonyl)-3-methyl-L-valyl-(4R)-N-{(1S)-1-cyano-2-[(3S)-2-oxopyrrolidin-3-yl]ethyl}-4-(trifluoromethyl)-L-prolinamide ethyl acetate (EtOAc) solvate.

FIG. 5: PXRD Pattern of N-(Methoxycarbonyl)-3-methyl-L-valyl-(4R)-N-{(1S)-1-cyano-2-[(3S)-2-oxopyrrolidin-3-yl]ethyl}-4-(trifluoromethyl)-L-prolinamide, Form 1.

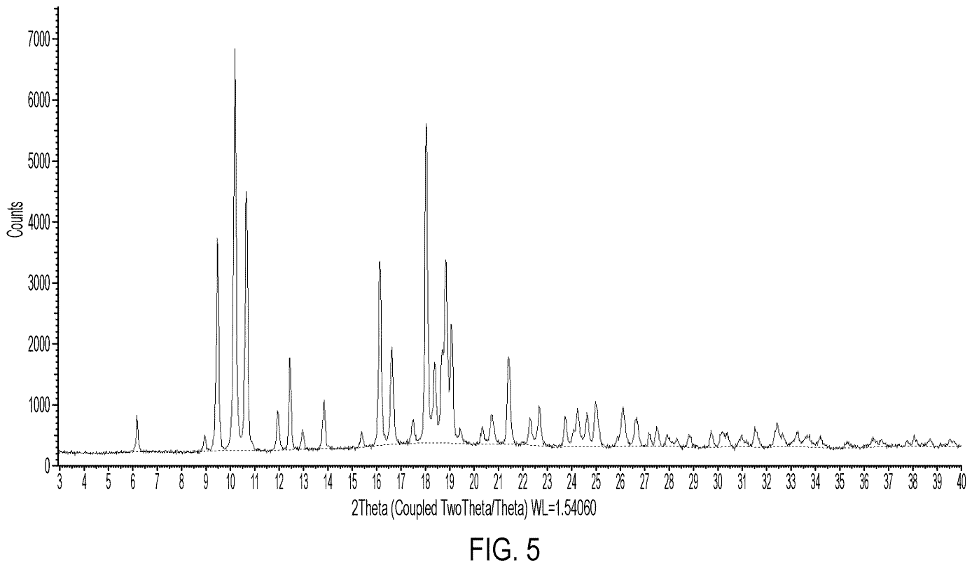

FIG. 6: Particle size and cumulative distribution for Example 3 LOT A (top) and LOT B (bottom).

FIG. 7: Particle size and cumulative distribution for Example 3 LOT C (top) and image of Compound I Form 1 co-processed with microcrystalline cellulose (90/10 w %/w %) (bottom).

FIG. 8: Particle size and cumulative distribution for Example 3 LOT D-Tray 1 (top) and LOT E (bottom).

FIG. 8A: PXRD pattern of Compound I Form 1 co-processed with 10 w % microcrystalline cellulose (MCC).

FIG. 8B: ¹³C solid state NMR spectrum of Compound I Form 1 co-processed with 10 w % MCC.

FIG. 8C: ¹⁹F solid state NMR spectrum of Compound I Form 1 co-processed with 10 wt % MCC.

FIG. 8D: Particle size distribution of different lots of Compound I, Form 1 (top: D[v,0.5]=5 μm and D[v,0.9]=26 μm multimodal distribution) and (bottom: D[v,0.5]=3 μm and D[v,0.9]=11 μm bimodal distribution with very fine particles).

FIG. 8E: Particle size distribution of a lot of Compound I, Form 1 with D[v,0.5]=76 μm and D[v,0.9]=200 μm—large particles with a bimodal distribution).

FIG. 8F: Particle size distribution of two lots of Compound I, Form 1 co-processed with 10 wt % MCC with D[v,0.5]=36 μm and D[v,0.9]=98 μm (top) and D[v,0.5]=73 μm and D[v,0.9]=165 μm (bottom)—both with a monomodal distribution.

FIG. 9: Images of Compound I Form 1 co-processed with microcrystalline cellulose (90/10 w %/w %) (top and bottom).

FIG. 10: PXRD Pattern of (2S,4R)-4-(Trifluoromethyl)pyrrolidine-2-carboxylic acid, Compound 14 free acid.

FIG. 11: PXRD Pattern of (2S,4R)-4-(Trifluoromethyl)pyrrolidine-2-carboxylic acid hydrochloride (1:1), Compound 14 HCl Salt—Pattern 1.

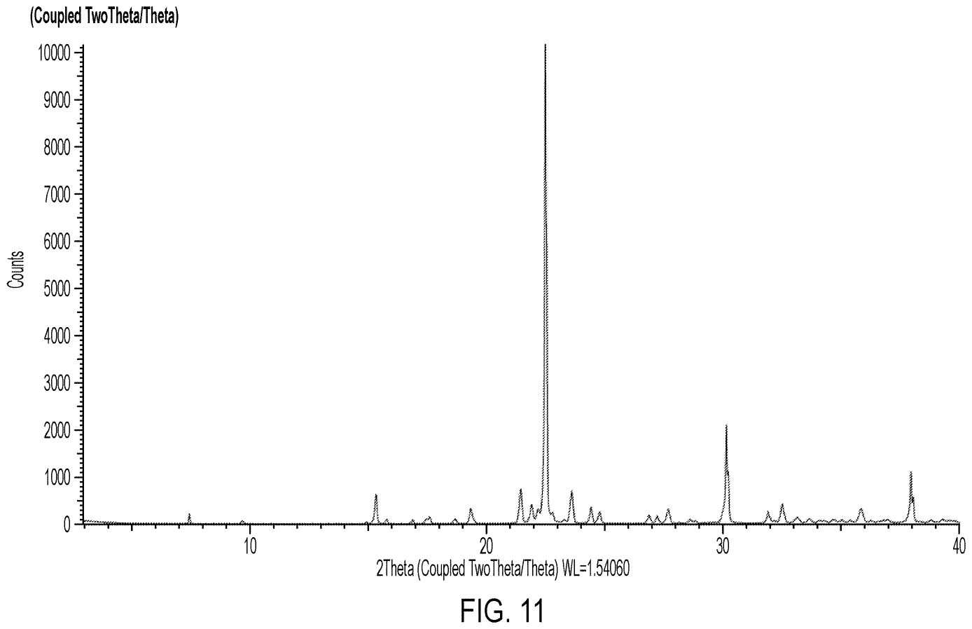

FIG. 12: PXRD Pattern of (2S,4R)-4-(Trifluoromethyl)pyrrolidine-2-carboxylic acid hydrochloride (1:1), Compound 14 HCl Salt—Pattern 2.

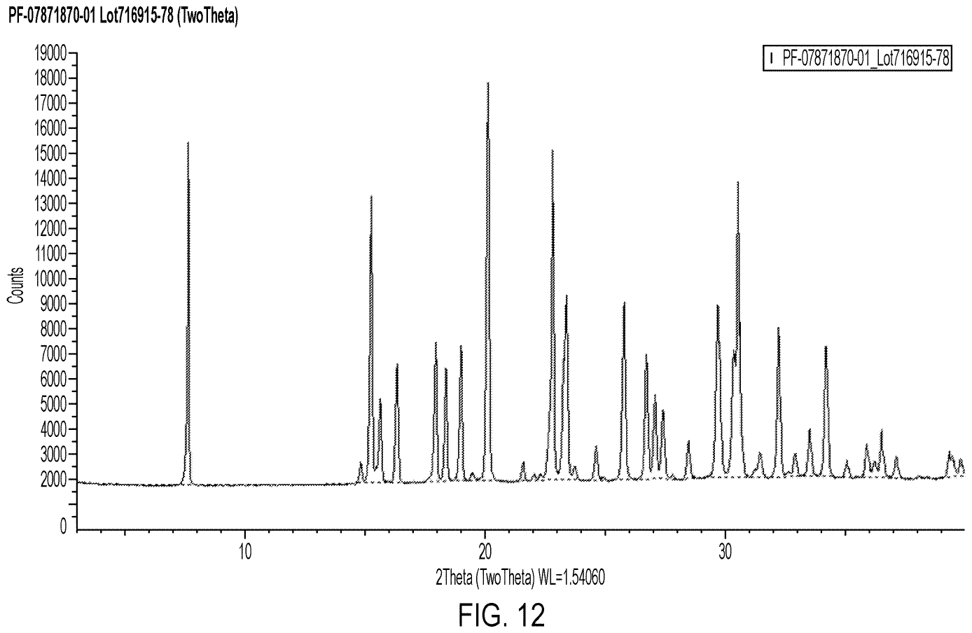

FIG. 13: PXRD Pattern of (2S)-2-[(Methoxycarbonyl)amino]-3,3-dimethylbutanoic acid, Compound 2.

FIG. 14: PXRD Pattern of tert-Butyl (2R,4R)-1-{(2S)-2-[(methoxycarbonyl)amino]-3,3-dimethylbutanoyl}-4-(trifluoromethyl)pyrrolidine-2-carboxylate, Compound 4.

FIG. 15: PXRD Pattern of (2S,4R)-1-{(2S)-2-[(methoxycarbonyl)amino]-3,3-dimethylbutanoyl}-4-(trifluoromethyl)pyrrolidine-2-carboxylic acid, Compound 6′ (free acid form).

FIG. 16: PXRD Pattern of Potassium (2S,4R)-1-{(2S)-2-[(methoxycarbonyl)amino]-3,3-dimethylbutanoyl}-4-(trifluoromethyl)pyrrolidine-2-carboxylate, Compound 6 (potassium salt form).

FIG. 17: PXRD Pattern of (2S)-2-[(Methoxycarbonyl)amino]-3,3-dimethylbutanoic acid, Compound 3 (free form).

FIG. 18: PXRD Pattern of (2S)-2-[(Methoxycarbonyl)amino]-3,3-dimethylbutanoic acid hydrochloride (1:1), Compound 3 hydrochloride.

FIG. 19: PXRD Pattern of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate, Compound 8.

FIG. 20: PXRD pattern of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate tartrate co-crystal

FIG. 21: PXRD pattern of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl) pyrrolidin-1-yl]-3,3-dimethyl-1-oxobutan-2-yl}carbamate maleate co-crystal

FIG. 22: PXRD pattern of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate succinate co-crystal

FIG. 23: PXRD pattern of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate fumarate co-crystal

FIG. 24: PXRD pattern of amorphous Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate

### DETAILED DESCRIPTION OF THE INVENTION

The present invention may be understood more readily by reference to the following detailed description and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular form “a”, “an”, and “the” include plural references unless indicated otherwise. For example, “a” substituent includes one or more substituents.

As used herein, the term “about” means within a statistically meaningful range of a value, such as a stated concentration range, time frame, molecular weight, particle size, temperature or pH. As used herein about means within 20%, preferably within 10%, and even more preferably within 5% of the indicated value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every whole number integer within the range is also contemplated as an embodiment of the invention.

The term, “solvate,” as used herein, refers to a crystal form of a substance which contains solvent. Particular solvates of Compound I include the ethyl acetate, isopropyl acetate and cyclopentyl methyl ether solvates of that compound. The term “hydrate” refers to a solvate wherein the solvent is water.

The term “seeding,” as used herein, means the addition of crystals to a crystallization system, for the purpose of initiating or enhancing nucleation or acting as substrate for further crystallization.

As used herein, the terms “API” or “active pharmaceutical ingredient” refer to anhydrous Methyl {(2S)-1-[(2S,4R)-2-({(1 S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate Form 1 (Compound I Form 1). Compound I Form 1 is a non-solvated anhydrous crystalline form.

Examples of characterizing identifiers for N-(Methoxycarbonyl)-3-methyl-L-valyl-(4R)-N-{(1 S)-1-cyano-2-[(3S)-2-oxopyrrolidin-3-yl]ethyl}-4-(trifluoromethyl)-L-prolinamide (Compound I Form 1) are provided below using a single method or a combination of instrument methods.

| Compound I | ¹⁹F ssNMR | ¹³C ssNMR | PXRD (°2θ) ± |
|---|---|---|---|
| Form 1 | (ppm) ± 0.2 ppm | (ppm) ± 0.2 ppm | 0.2 °2θ |
| Single | | 50.8 | |
| Single | | 50.8, 58.3, 43.5 | |
| Combination | −70.7 | 50.8 | |
| Combination | −70.7 | 50.8, 58.3, 43.5 | |
| Combination | | 50.8 | at least one: 9.1, |
| | | | 9.6, 10.3, 16.2 |
| Combination | | 50.8, 58.3, 43.5 | at least one: 9.1, |
| | | | 9.6, 10.3, 16.2 |
| Combination | −70.7 | 50.8 | at least one: 9.1, |
| | | | 9.6, 10.3, 16.2 |
| Combination | −70.7 | 50.8, 58.3, 43.5 | at least one: 9.1, |
| | | | 9.6, 10.3, 16.2 |

Compound I Form 1 is characterized by any one of the following characteristic peaks or combinations of peaks: a ¹³C solid state NMR peak at 50.8 ppm±0.2 ppm;
¹³C solid state NMR peaks at 50.8 ppm and 58.3 ppm, wherein each peak is ±0.2 ppm;
¹³C solid state NMR peaks at 50.8 ppm and 43.5 ppm, wherein each peak is ±0.2 ppm;
¹³C solid state NMR peaks at 50.8 ppm, 58.3 ppm and 43.5 ppm, wherein each peak is ±0.2 ppm;
¹³C solid state NMR peak at 50.8 ppm±0.2 ppm and a ¹⁹F solid state NMR peak at −70.7 ppm±0.2 ppm;
¹³C solid state NMR peaks at 50.8 ppm and 58.3 ppm, wherein each peak is ±0.2 ppm and a ¹⁹F solid state NMR peak at −70.7 ppm±0.2 ppm;
¹³C solid state NMR peaks at 50.8 ppm and 43.5 ppm, wherein each peak is ±0.2 ppm and a ¹⁹F solid state NMR peak at −70.7 ppm±0.2 ppm;
¹³C solid state NMR peaks at 50.8 ppm, 58.3 ppm and 43.5 ppm, wherein each peak is ±0.2 ppm and a ¹⁹F solid state NMR peak at −70.7 ppm±0.2 ppm;
¹³C solid state NMR peak at 50.8 ppm±0.2 ppm and one to four powder X-ray diffraction peaks (Cu Kα radiation) selected from the group consisting of peaks at 9.1, 9.6, 10.3, and 16.2 degrees 2θ wherein each peak is ±0.2 degrees 2θ;
¹³C solid state NMR peaks at 50.8 ppm and 58.3 ppm wherein each peak is ±0.2 ppm and one to four powder X-ray diffraction peaks (Cu Kα radiation) selected from the group consisting of peaks at 9.1, 9.6, 10.3 and 16.2 degrees 2θ wherein each peak is ±0.2 degrees 2θ;
¹³C solid state NMR peaks at 50.8 ppm and 43.5 ppm wherein each peak is ±0.2 ppm and one to four powder X-ray diffraction peaks (Cu Kα radiation) selected from the group consisting of peaks at 9.1, 9.6, 10.3 and 16.2 degrees 2θ wherein each peak is ±0.2 degrees 2θ;
¹³C solid state NMR peaks at 50.8 ppm, 58.3 ppm and 43.5 ppm wherein each peak is ±0.2 ppm and one to four powder X-ray diffraction peaks (Cu Kα radiation) selected from the group consisting of peaks at 9.1, 9.6, 10.3 and 16.2 degrees 2θ wherein each peak is ±0.2 degrees 2θ;
¹³C solid state NMR peak at 50.8 ppm±0.2 ppm, a ¹⁹F solid state NMR peak at −70.7 ppm±0.2 ppm and one to four powder X-ray diffraction peaks (Cu Kα radiation) selected from the group consisting of peaks at 9.1, 9.6, 10.3 and 16.2 degrees 2θ wherein each peak is ±0.2 degrees 2θ;
¹³C solid state NMR peaks at 50.8 ppm and 58.3 ppm wherein each peak is ±0.2 ppm, a ¹⁹F solid state NMR peak at −70.7 ppm±0.2 ppm and one to four powder X-ray diffraction peaks (Cu Kα radiation) selected from the group consisting of peaks at 9.1, 9.6, 10.3 and 16.2 degrees 2θ wherein each peak is ±0.2 degrees 2θ;
¹³C solid state NMR peaks at 50.8 ppm and 43.5 ppm wherein each peak is ±0.2 ppm, a ¹⁹F solid state NMR peak at −70.7 ppm±0.2 ppm and one to four powder X-ray diffraction peaks (Cu Kα radiation) selected from the group consisting of peaks at 9.1, 9.6, 10.3 and 16.2 degrees 2θ wherein each peak is ±0.2 degrees 2θ; and
¹³C solid state NMR peaks at 50.8 ppm, 58.3 ppm and 43.5 ppm wherein each peak is ±0.2 ppm, a ¹⁹F solid state NMR peak at −70.7 ppm±0.2 ppm and one to four powder X-ray diffraction peaks (Cu Kα radiation) selected from the group consisting of peaks at 9.1, 9.6, 10.3 and 16.2 degrees 2θ wherein each peak is ±0.2 degrees 2θ.

In the embodiments of the invention Compound I Form 1 can be characterized by any one of the preceding listed peaks or combinations of peaks.

The term “peptide coupling agent” as used herein refer to agents used to couple compounds such as a carboxylic acid or carboxylate with an amine to form an amide bond. Peptide coupling agents include but are not limited to those as described in Dunetz, J. R., Magano, J., Weisenburger, G. A. Org. Process Res. Dev. 2016, 20, 140-177. Representative peptide coupling agents used in the instant processes include but are not limited to 2-chloro-1-methylpyridinium p-toluenesulfonate, a combination of 2-hydroxy pyridine N-oxide and 1-ethyl-3-(3-dimethylaminopropyl)carbodiimide and a combination of 2-hydroxypyridine N-oxide and 1-ethyl-3-(3-dimethylaminopropyl) carbodiimide hydrochloride. Alternatively, an agent used to convert a carboxylic acid to an acid chloride, such as methanesulfonyl chloride, can be employed as a “peptide coupling agent”. The resulting acid chloride reacts with an amine to form an amide bond.

The term “dehydrating agent” as used herein refer to agents used to dehydrate a primary amide group to the corresponding nitrile group. Dehydrating agents include but are not limited to those described in Ganesan, M., Nagaraaj, P. Org. Chem. Front. 2020, 7 3792-3814 and Talbi, I. et al ACS Omega 2018, 3, 5078-5082. Representative dehydrating agents used in the instant processes include but are not limited to trifluoroacetic anhydride, propane phosphonic acid anhydride, triphenylphosphite, diethyl chlorophosphate, ethyl dichlorophosphate, phosphorus trichloride and tris-(dimethylamino)phosphine.

The term “base comprising M⁺” as used in certain embodiments herein refer to an alkali metal alkoxide, alkali metal hydroxide or alkali metal disilazide compound wherein the alkali metal is potassium, sodium or lithium. Representative “base comprising M⁺” compounds include but are not limited to sodium tert-butoxide, sodium tert-amylate, potassium methoxide, potassium hydroxide, potassium tert-butoxide, potassium tert-amylate and potassium hexamethyldisilazide. When the alkali metal is potassium the compound is a “base comprising K+”.

The term “base” as used in certain embodiments herein includes but is not limited to organic amine bases including but not limited to N-methylimidazole, N-methyl morpholine, diethylamine, triethylamine and 1,8-diazabicyclo(5.4.0)undec-7-ene.

As used herein, the term “primary particles” refers to individual API crystals.

As used herein, the term “agglomerates” refers to tightly bound API crystals that are difficult to disperse into primary particles during processing and particle size analysis.

In particle size determinations, the median value is defined as the value where half of the population resides above this point, and half resides below this point. For particle size distributions the median is called the D[50]. The D[50] is the size in microns that splits the distribution with half above and half below this diameter. The expression Dv50 or D[v,0.5] is sometimes used for the median of a volume distribution.

As used herein, the term D[90] means that 90% of the total particles are smaller than that size.

As used herein, the term D[50] means that 50% of the total particles are smaller than that size.

As used herein, the term D[10] means that 10% of the total particles are smaller than that size.

As used herein, the term D[4,3] means the mean particle diameter over volume (the DeBroukere mean). The volume moment mean is relevant for many samples as it reflects the size of those particles which constitute the bulk of the sample volume. It is most sensitive to the presence of large particulates in the size distribution.

As used herein, the term Aspect Ratio 50 or AR50 means the volume at which of 50% of the particles are larger than and 50% of the particles are smaller than.

As used herein, the term Bulk density is the ratio of the mass per unit volume of a loose powder, typically expressed in g/mL or g/cm³.

As used herein, the term Tapped density is the ratio of the mass per unit volume of a powder after it has been tapped for a period of time, typically expressed in g/mL or g/cm³.

The bulk density and tapped density can be determined using methods such as those described in US Pharmacopeia <616> BULK DENSITY (USP 1 May 2024) OF POWDERS.

In some embodiments Compound I Form 1 has a D[90] value of less than 200 μM, a D[50] value of less than 80 μM and a D[10] value of less than 20 μM. In some such embodiments, the Compound I Form 1 has a D[10] value of from about 10 μm to about 15 μm. In other such embodiments, the Compound I Form 1 has a D[90] value of from about 160 μm to about 190 μm. In other such embodiments, the Compound I Form 1 has a D[50] value of from about 50 μm to about 70 μm. In other embodiments the Compound I Form 1 has a D[4,3] value of from about 80 μm to about 90 μm. In some such embodiments, the Compound I Form 1 has a D[10] value of from about 5 μm to about 20 μm and a D[90] value of from about 150 μm to about 200 μm. In further embodiments, the Compound I Form 1 has a D[10] value of from about 5 μm to about 20 m, a D[90] value of from about 150 μm to about 200 m, and a D[50] value of from about 40 μm to about 80 μm.

In another aspect, the invention provides Compound I, Form 1 having a primary particle size distribution having at least one of:
(a) a D[10] value of from about 5 μm to about 25 μm;
(b) a D[50] value of from about 50 μm to about 75 μm;
(c) a D[90] value of from about 165 μm to about 190 μm; and
(d) a D[4,3] value of from about 75 μm to about 95 μm.

Each of the foregoing values of embodiments for D[10] can be combined with any value for D[50] and/or D[90] value not inconsistent with it. Each of the foregoing values of embodiments for D[50] can be combined with any value for D[10] and/or D[90] value not inconsistent with it. Each of the foregoing values of embodiments for D[90] can be combined with any value for D[10] and/or D[50] value not inconsistent with it.

The following embodiments, E1 to E73 are representative embodiments of the present invention which should be construed in a non-limiting manner.
E1 is a process for preparing a (2S,4R)-1-{(2S)-2-[(methoxycarbonyl)amino]-3,3-dimethylbutanoyl}-4-(trifluoromethyl)pyrrolidine-2-carboxylate M⁺ salt (Compound 6a) of formula:

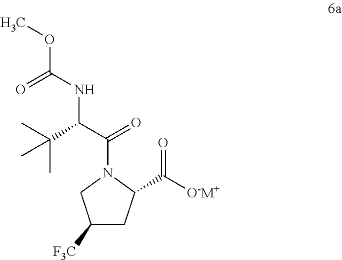

comprising the steps of
a) combining tert-butyl (2R,4R)-1-{(2S)-2-[(methoxycarbonyl)amino]-3,3-dimethylbutanoyl}-4-(trifluoromethyl)pyrrolidine-2-carboxylate (Compound 4) a solvent and water; and
b) adding a base comprising M⁺ to the mixture from step a) to generate intermediate tert-Butyl (2S,4R)-1-{(2S)-2-[(methoxycarbonyl)amino]-3,3-dimethylbutanoyl}-4-(trifluoromethyl)pyrrolidine-2-carboxylate, Compound 5, in situ which further reacts to provide the (2S,4R)-1-{(2S)-2-[(methoxycarbonyl)amino]-3,3-dimethylbutanoyl}-4-(trifluoromethyl)pyrrolidine-2-carboxylate M⁺ salt, Compound 6a

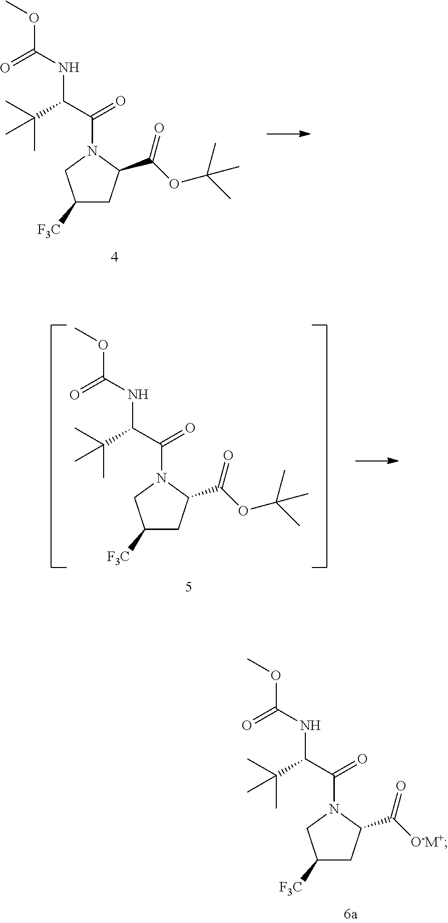

wherein M⁺ is selected from Li⁺, Na⁺ and K⁺.
E2 is the process of E1 wherein the base comprising M⁺ is selected from sodium tert-butoxide, sodium tert-amylate, potassium methoxide, potassium hydroxide, potassium tert-butoxide, potassium tert-amylate and potassium hexamethyldisilazide.
E3 is the process of E1 or E2 wherein the base comprising M⁺ is potassium tert-butoxide or potassium tert-amylate.
E4 is the process of any one of E1 to E3 wherein the base comprising M⁺ is potassium tert-butoxide.
E5 is the process of any one of E1 to E4 wherein the solvent is selected from the group consisting of methyl tert-butyl ether, tetrahydrofuran, isopropyl alcohol, tert-amyl alcohol, acetonitrile, tert-butanol and 2-methyl tetrahydrofuran.
E6 is the process of E1 for preparing potassium (2S,4R)-1-{(2S)-2-[(methoxycarbonyl)amino]-3,3-dimethylbutanoyl}-4-(trifluoromethyl)pyrrolidine-2-carboxylate, Compound 6

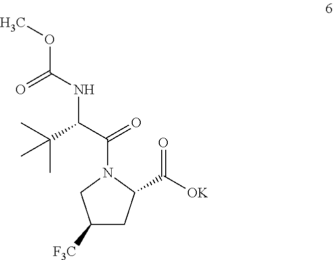

comprising the steps of
a) combining tert-Butyl (2R,4R)-1-{(2S)-2-[(methoxycarbonyl)amino]-3,3-dimethylbutanoyl}-4-(trifluoromethyl)pyrrolidine-2-carboxylate, Compound 4, a solvent and water; and
b) adding a base comprising K⁺ to the mixture from step a) to generate intermediate tert-Butyl (2S,4R)-1-{(2S)-2-[(methoxycarbonyl)amino]-3,3-dimethylbutanoyl}-4-(trifluoromethyl)pyrrolidine-2-carboxylate, Compound 5, in situ which further reacts to provide potassium (2S,4R)-1-{(2S)-2-[(methoxycarbonyl)amino]-3,3-dimethylbutanoyl}-4-(trifluoromethyl)pyrrolidine-2-carboxylate, Compound 6

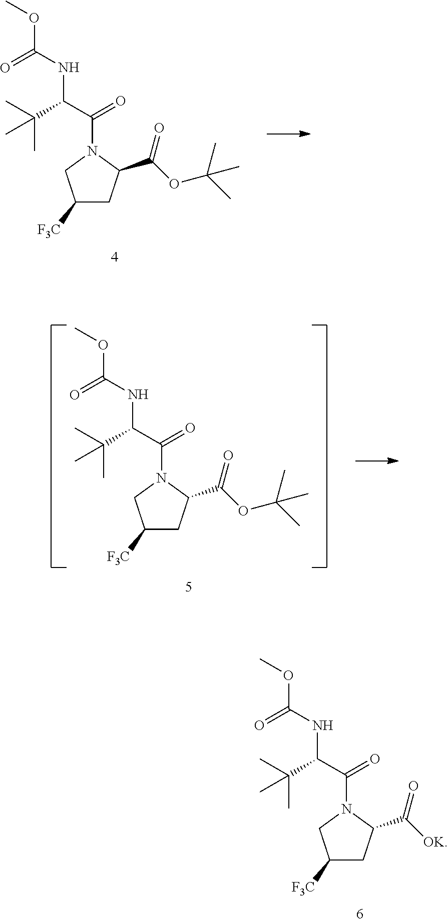

E7 is the process of E6 wherein the base comprising K⁺ is selected from the group consisting of potassium tert-butoxide, potassium tert-amylate, potassium methoxide, potassium hydroxide and potassium hexamethyldisilazide.
E8 is the process of E6 or E7 wherein the base comprising K⁺ is potassium tert-butoxide.
E9 is the process of any one of E6 to E8 wherein the solvent is selected from the group consisting of methyl tert-butyl ether, tetrahydrofuran, isopropyl alcohol, tert-amyl alcohol, acetonitrile, tert-butanol and 2-methyl tetrahydrofuran.
E10 is the process of any one of E6 to E9 wherein in step a) 1.0 equivalent of tert-Butyl (2R,4R)-1-{(2S)-2-[(methoxycarbonyl)amino]-3,3-dimethylbutanoyl}-4-(trifluoromethyl) pyrrolidine-2-carboxylate, Compound 4 is combined with 1 to 10 volumes of a solvent and 1.2 equivalents of water and in step b) 1.2 equivalents of potassium tert-butoxide is added.
E11 is the process of any one of E6 to E10 wherein in step a) 1.0 equivalent of tert-Butyl (2R,4R)-1-{(2S)-2-[(methoxycarbonyl)amino]-3,3-dimethylbutanoyl}-4-(trifluoromethyl)pyrrolidine-2-carboxylate, Compound 4 is combined with 2.5 volumes of the solvent methyl tert-butyl ether (MTBE) and 1.2 equivalents of water at ambient temperature and in step b) 1.2 equivalents of potassium tert-butoxide (20 weight % in THF) is added at ambient temperature.
E12 is the process of E11 further comprising step c) stirring the reaction mixture obtained from step b) at ambient temperature for a period of at least 12 hours.
E13 is the process of E12 further comprising step d) addition of 0.5 volumes of methanol with stirring after step c) to provide a reaction mixture slurry.
E14 is the process of E13 further comprising step e) filtering the reaction mixture slurry obtained from step d) to isolate a solid comprising potassium (2S,4R)-1-{(2S)-2-[(methoxycarbonyl)amino]-3,3-dimethylbutanoyl}-4-(trifluoromethyl)pyrrolidine-2-carboxylate, Compound 6.
E15 is the process of E14 further comprising step f) washing the solid comprising potassium (2S,4R)-1-{(2S)-2-[(methoxycarbonyl)amino]-3,3-dimethylbutanoyl}-4-(trifluoromethyl)pyrrolidine-2-carboxylate, Compound 6 obtained from step e) with a mixture of MTBE (1.7 volumes) and methanol (0.3 volumes).
E16 is the process of E15 further comprising step g) drying the solid obtained in step f) comprising potassium (2S,4R)-1-{(2S)-2-[(methoxycarbonyl)amino]-3,3-dimethylbutanoyl}-4-(trifluoromethyl) pyrrolidine-2-carboxylate, Compound 6.
E17 is the process of any one of E14 to E16 wherein the isolated solid comprises greater than or equal to 90% by weight of crystalline potassium (2S,4R)-1-{(2S)-2-[(methoxycarbonyl) amino]-3,3-dimethylbutanoyl}-4-(trifluoromethyl)pyrrolidine-2-carboxylate, Compound 6.
E18 is the process of E17 wherein the isolated solid comprises less than 10% by weight of potassium (2R,4R)-1-{(2S)-2-[(methoxycarbonyl)amino]-3,3-dimethyl butanoyl}-4-(trifluoromethyl)pyrrolidine-2-carboxylate.
E19 is the process of E17 wherein the isolated solid comprises greater than or equal to 95% by weight of crystalline potassium (2S,4R)-1-{(2S)-2-[(methoxycarbonyl)amino]-3,3-dimethylbutanoyl}-4-(trifluoromethyl)pyrrolidine-2-carboxylate, Compound 6.
E20 is the process of E19 wherein the isolated solid comprises less than 5% by weight of potassium (2R,4R)-1-{(2S)-2-[(methoxycarbonyl)amino]-3,3-dimethyl butanoyl}-4-(trifluoromethyl)pyrrolidine-2-carboxylate.
E21 is the process of E19 wherein the isolated solid comprises greater than or equal to 98% by weight of crystalline potassium (2S,4R)-1-{(2S)-2-[(methoxycarbonyl)amino]-3,3-dimethylbutanoyl}-4-(trifluoromethyl)pyrrolidine-2-carboxylate, Compound 6.
E22 is the process of E19 wherein the isolated solid comprises less than 2% by weight of potassium (2R,4R)-1-{(2S)-2-[(methoxycarbonyl)amino]-3,3-dimethyl butanoyl}-4-(trifluoromethyl)pyrrolidine-2-carboxylate.
E23 is a process for preparing a solvate of Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate (Compound I)

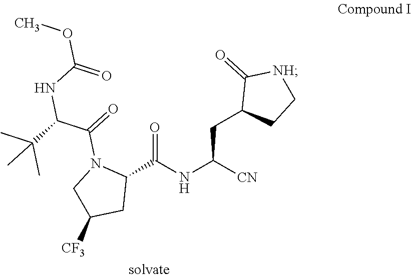 [SMILES: COC(=O)N[C@H](C(=O)N1C[C@H](C(F)(F)F)C[C@H]1C(=O)N[C@H](C#N)C[C@@H]1CCNC1=O)C(C)(C)C]

comprising the steps of
h) reacting (2S,4R)-1-{(2S)-2-[(methoxycarbonyl)amino]-3,3-dimethylbutanoyl}-4-(trifluoromethyl)pyrrolidine-2-carboxylic acid, Compound 6′, or a pharmaceutically acceptable salt thereof with (2S)-2-Amino-3-[(3S)-2-oxopyrrolidin-3-yl]propanamide, Compound 7′, or a pharmaceutically acceptable salt thereof in the presence of a peptide coupling reagent, a base and a solvent

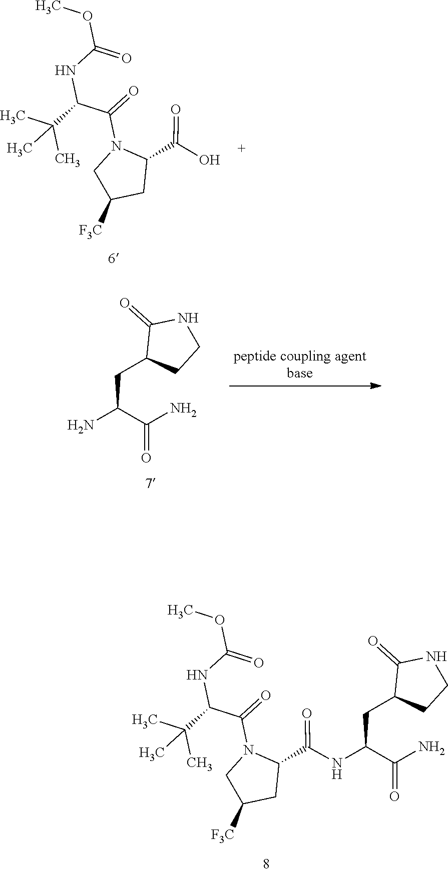 [SMILES: COC(=O)N[C@H](C(=O)N1C[C@H](C(F)(F)F)C[C@H]1C(=O)O)C(C)(C)C ; NC(=O)[C@@H](N)C[C@@H]1CCNC1=O ; COC(=O)N[C@H](C(=O)N1C[C@H](C(F)(F)F)C[C@H]1C(=O)N[C@@H](C[C@@H]1CCNC1=O)C(N)=O)C(C)(C)C]

to provide methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate, Compound 8; and
i) reacting Compound 8 with a dehydrating reagent

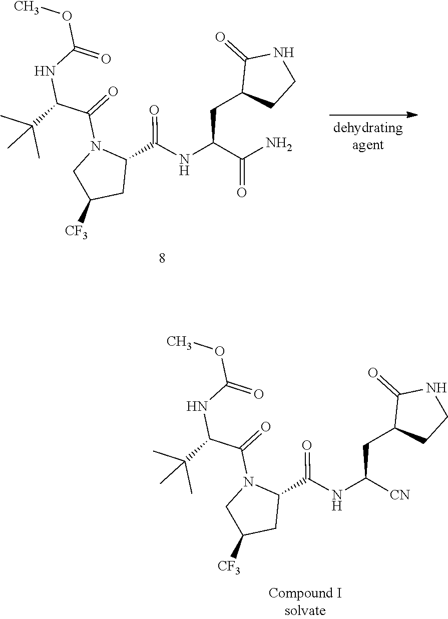 [SMILES: COC(=O)N[C@H](C(=O)N1C[C@H](C(F)(F)F)C[C@H]1C(=O)N[C@@H](C[C@@H]1CCNC1=O)C(N)=O)C(C)(C)C ; COC(=O)N[C@H](C(=O)N1C[C@H](C(F)(F)F)C[C@H]1C(=O)N[C@H](C#N)C[C@@H]1CCNC1=O)C(C)(C)C]

to provide a solvate of methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate, Compound I solvate.
E24 is the process of E23 wherein in step h) the peptide coupling reagent is selected from 2-chloro-1-methylpyridinium p-toluenesulfonate, a combination of 2-hydroxy pyridine N-oxide and 1-ethyl-3-(3-dimethylaminopropyl)carbodiimide and a combination of 2-hydroxypyridine N-oxide and 1-ethyl-3-(3-dimethylaminopropyl) carbodiimide hydrochloride and the base is triethylamine, N-methyl morpholine or N,N-diisopropylethylamine.
E24′ is the process of E23 or E24 wherein Compound 6 is prepared according to any one of E1 to E22.
E25 is the process of E23 or E24 wherein the solvent in step h) is methyl ethyl ketone or isopropyl acetate.
E26 is the process of E25 wherein in step i) the dehydrating agent is selected from trifluoroacetic anhydride, propane phosphonic acid anhydride, triphenylphosphite, diethyl chlorophosphate, ethyl dichlorophosphate, phosphorus trichloride and tris-(dimethylamino)phosphine in the presence of a base selected from N-methylimidazole, N-methyl morpholine, diethylamine, triethylamine and 1,8-Diazabicyclo(5.4.0)undec-7-ene.
E27 is the process of E26 wherein in step i) the dehydrating agent is trifluoroacetic anhydride or propane phosphonic acid anhydride.
E28 is the process of any one of E23 to E27 wherein isopropyl acetate is used as the solvent in steps h) and i) and the reaction mixture from step i) is concentrated and to it is added heptane to provide methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate isopropyl acetate solvate, Compound I IPAc solvate.
E29 is the process of any one of E23 to E27 wherein in step h) the solvent is methyl ethyl ketone, the peptide coupling agent is a combination of 2-hydroxypyridine N-oxide and 1-ethyl-3-(3-dimethylaminopropyl)carbodiimide and the base is triethylamine.
E30 is the process of E29 wherein after the coupling reaction is complete the reaction mixture from step h) the solvent is exchanged to isopropyl acetate by addition of isopropyl acetate and distillation and in step i) the dehydrating agent is trifluoroacetic anhydride and the base is N-methyl morpholine.
E31 is the process of E30 wherein the reaction mixture from step i) is quenched with aqueous ammonium hydroxide, the layers are separated and the isopropyl acetate layer is solvent exchanged into cyclopentyl methyl ether by addition of cyclopentyl methyl ether and distillation.
E32 is the process of E31 wherein the cyclopentyl methyl ether mixture from step i) is cooled to 10 °C and stirred at 10 °C for one hour or longer and methyl {(2S)-1-[(2S,4R)-2-({(1 S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate cyclopentyl methyl ether solvate, Compound I CPME solvate is isolated.
E33 is a process for preparing methyl {(2S)-1-[(2S,4R)-2-({(1S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate, Compound I Form 1 polymorph comprising the step of combining the ethyl acetate solvate, isopropyl acetate solvate or methoxycyclopentane solvate of methyl {(2S)-1-[(2S,4R)-2-({(1S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate with heptane and heating the mixture to a temperature range of 50 °C to 100 °C and stirring the mixture to provide methyl {(2S)-1-[(2S,4R)-2-({(1S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate, Compound I Form 1.
E33′ is the process of E33 wherein the ethyl acetate solvate, isopropyl acetate solvate or methoxycyclopentane solvate of methyl {(2S)-1-[(2S,4R)-2-({(1S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate are prepared according to the process of E23 to E32.
E34 is the process of E33 wherein the mixture is stirred at 60 °C to 80 °C for a period of 6 hours to 24 hours.
E35 is the process of E34 wherein the mixture is stirred at about 70 °C for 6 hours to 12 hours then the mixture is cooled to 20 °C over 4 hours and held at 20 °C for 2 hours then the methyl {(2S)-1-[(2S,4R)-2-({(1S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate, Compound I Form 1 is isolated.
E36 is a process for preparing methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate ethyl acetate solvate (Compound I, ethyl acetate solvate)

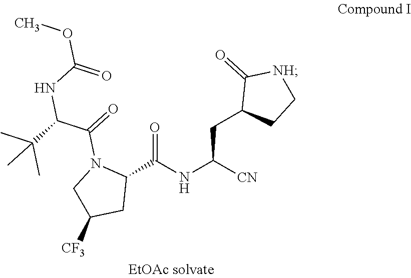 [SMILES: COC(=O)N[C@H](C(=O)N1C[C@H](C(F)(F)F)C[C@H]1C(=O)N[C@H](C#N)C[C@@H]1CCNC1=O)C(C)(C)C]

comprising the steps of
   h′) reacting potassium (2S,4R)-1-{(2S)-2-[(methoxycarbonyl)amino]-3,3-dimethylbutanoyl}-4-(trifluoromethyl)pyrrolidine-2-carboxylate, Compound 6 with (2S)-2-amino-3-[(3S)-2-oxopyrrolidin-3-yl]propanamide hydrochloride (1:1), Compound 7 in the presence of 2-chloro-1-methylpyridinium p-toluenesulfonate and a base

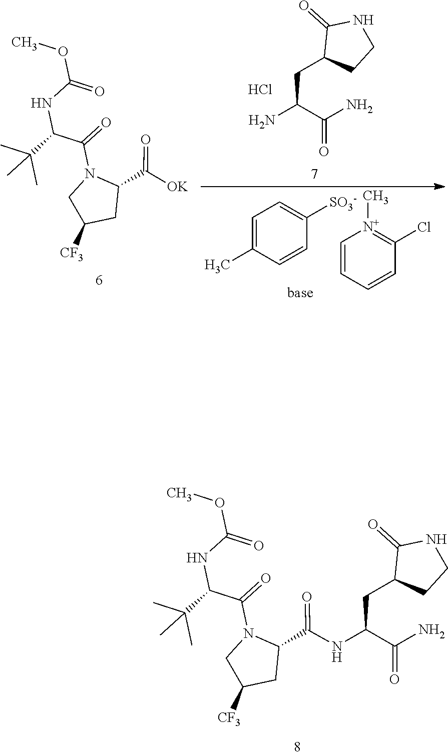

to provide methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate, Compound 8; and
   i′) reacting Compound 8 with trifluoroacetic anhydride in the presence of N-methylimidazole in ethyl acetate

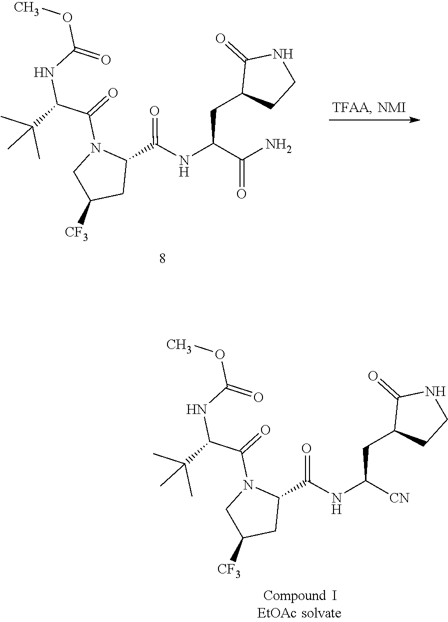

to provide methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate ethyl acetate solvate, Compound I EtOAc solvate.
E37 is the process of E36 wherein step h′) is carried out in methyl ethyl ketone and water.
E38 is the process of E37 wherein 1.0 equivalent of potassium (2S,4R)-1-{(2S)-2-[(methoxycarbonyl)amino]-3,3-dimethylbutanoyl}-4-(trifluoromethyl)pyrrolidine-2-carboxylate, Compound 6 is reacted with 1.15 equivalents of (2S)-2-Amino-3-[(3S)-2-oxopyrrolidin-3-yl]propanamide hydrochloride (1:1), Compound 7 in the presence of 1.2 equivalents of 2-chloro-1-methylpyridinium p-toluenesulfonate and the base is 3.0 equivalents of N-methyl morpholine.
E39 is the process of E38 where the reaction mixture in step h′) is stirred for 4 hours at 20 °C.
E40 is the process of E39 wherein the crude reaction mixture from step h′) is solvent exchanged into ethyl acetate by vacuum distillation until the water content is less than 0.2% by weight to provide a slurry of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate, Compound 8 in ethyl acetate.
E41 is the process of E40 wherein in step i′) the slurry of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate, Compound 8 in ethyl acetate is cooled to 0 °C and 5.0 equivalents of N-methylimidazole is added followed by addition of 2.5 equivalents of trifluoroacetic anhydride over 30 minutes then the reaction mixture is stirred at 0 °C for 1 hour.
E42 is the process of E41 further comprising step j) quenching the reaction mixture from step i′) with an aqueous mixture of citric acid monohydrate and sodium chloride and the resulting layers are separated and the aqueous layer is back extracted with ethyl acetate.
E43 is the process of E42 wherein the combined organic ethyl acetate layers are concentrated in vacuo to approximately half the original volume, heptane is added then the methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate ethyl acetate solvate is isolated by filtration.
E44 is the process of E43 further comprising the step of combining the methyl {(2S)-1-[(2S,4R)-2-({(1S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate ethyl acetate solvate with heptane and the mixture is stirred at 60 °C to 80 °C for a period of 6 hours to 24 hours to provide methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate, Compound I Form 1.
E45 is the process of E44 wherein the mixture is stirred at about 70 °C for 6 hours to 12 hours then the mixture is cooled to 20 °C over 4 hours and held at 20 °C for 2 hours then the methyl {(2S)-1-[(2S,4R)-2-({(1S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate, Compound I Form 1 is isolated.
E46 is a process for preparing a mixture of methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 and microcrystalline cellulose (MCC) or microcrystalline cellulose-SiO₂ (MCC-SiO₂), the process comprising the steps (k) to (q):
(k) dissolving methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 in ethyl acetate and heptane;
   (l) seeding the solution obtained in step (k) with about 10 weight % to about 50 weight % of microcrystalline cellulose or microcrystalline cellulose-SiO₂ wherein the weight % is the % weight of the microcrystalline cellulose or microcrystalline cellulose-SiO₂ to the combined weight of the microcrystalline cellulose or microcrystalline cellulose-SiO₂ and Compound I Form 1;
   (m) adding heptane to the mixture from step (l);
   (n) removing and replacing the ethyl acetate present in the mixture from step (m) with heptane by a constant volume distillation until the level of ethyl acetate is below 4 weight % of total solvent composition to obtain a slurry;
   (o) the slurry from step (n) is stirred at 70 °C or more for at least 6 hours and is then cooled to ambient temperature;
   (p) isolating the resulting methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 and microcrystalline cellulose or microcrystalline-SiO₂ mixture; and
   (q) drying the product from step (p).
E46′ is the process of E46 wherein the methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 is prepared according to any one of E33 to E35 or E44 to E45.
E47 is the process of E46 wherein:
   (k) methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 is dissolved in ethyl acetate (6 L/Kg of Form 1) and heptane (2 L/kg of Form 1) at 60 °C;
   (l) the solution is cooled to 45 °C and seeded with 10 weight % of microcrystalline cellulose;
   (m) heptane is added to the mixture from step (l) over a period of 4 hours wherein the amount of heptane added is about 7 L heptane/Kg of Form 1 and cooling the mixture to 20 °C in not less than 3 hours;
   (n) the ethyl acetate in the mixture from step (c) is removed and replaced with heptane, by constant volume distillation (15 L solvent/Kg of Form 1) until the level of ethyl acetate is below 4 weight % of total solvent composition to obtain a slurry;
   (o) the slurry from step (n) is stirred at 80 °C for at least 6 hours and then cooled to 20 °C over 5 hours;
   (p) isolating the resulting methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 and microcrystalline cellulose mixture (90 weight %/10 weight %); and
   (q) drying the solid mixture from step (p) at 80 °C for at least 12 hours.
E48 is the process of E46 or E47 wherein the solid mixture obtained from step (g) comprises methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 with a D[90] value of less than 200 μM, a D[50] value of less than 80 μM and a D[10] value of less than 20 μM.
E49 is the process of E48 wherein the Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 has a D[90] value of about 160 μM to about 190 μM.
E50 is the process of E48 or E49 wherein the methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 has a D[50] value of about 50 μM to about 70 μM.
E51 is the process of any one of E48 to E50 wherein the methyl {(2S)-1-[(2S,4R)-2-({(1 S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 has a particle size distribution D[10] of about 10 μM to about 15 μM.
E52 is the process of any one of E48 to E51 wherein the methyl {(2S)-1-[(2S,4R)-2-({(1 S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 has a D[4,3] value of about 80 μM to about 90 μM.
E53 is the process of any one of E48 to E52 wherein the methyl {(2S)-1-[(2S,4R)-2-({(1 S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate Form 1 has an aspect ratio (AR50) of 0.6 to 0.7, a bulk density of 0.35 g/cm³ to 0.40 g/cm³ and a tapped density of 0.45 g/cm³ to 0.55 g/cm³.
E54 is a composition comprising methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 and microcrystalline cellulose wherein the methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 has a D[90] value of less than 200 μM, a D[50] value of less than 80 μM and a D[10] value of less than 20 μM.
E55 is the composition of E54 which is 90 weight % methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 and 10 weight % microcrystalline cellulose.
E56 is the composition of claim E54 or E55 wherein the methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 has a D[90] value of about 160 μM to about 190 μM.
E57 is the composition of any one of E54 to E56 wherein the methyl {(2S)-1-[(2S,4R)-2-({(1S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate Form 1 has a D[50] value of about 50 μM to about 70 μM.
E58 is the composition of any one of E54 to E57 wherein the methyl {(2S)-1-[(2S,4R)-2-({(1S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate Form 1 has a D[10] value of about 10 μM to about 15 μM.
E59 is the composition of any one of E54 to E58 wherein the methyl {(2S)-1-[(2S,4R)-2-({(1S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate Form 1 has a D[4,3] value of about 80 μM to about 90 μM.
E60 is the composition of any one of E54 to E59 wherein the Methyl {(2S)-1-[(2S,4R)-2-({(1S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate Form 1 has an aspect ratio (AR50) of 0.6 to 0.7, a bulk density of 0.35 g/cm³ to 0.40 g/cm³ and a tapped density of 0.45 g/cm³ to 0.55 g/cm³.
E61 is the compound (2S,4R)-1-{(2S)-2-[(methoxycarbonyl)amino]-3,3-dimethylbutanoyl}-4-(trifluoromethyl)pyrrolidine-2-carboxylic acid; or a salt thereof.
E62 is the compound of E61 wherein the salt is selected from lithium, sodium and potassium.
E63 is the compound of E62 which is potassium (2S,4R)-1-{(2S)-2-[(methoxycarbonyl) amino]-3,3-dimethylbutanoyl}-4-(trifluoromethyl)pyrrolidine-2-carboxylate.
E64 is a composition comprising 90 wt % methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 and 10 wt % microcrystalline cellulose wherein the methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 has a D[90] value of 95 μM to 240 μM.
E65 is the composition of E64 wherein the methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 has a D[50] value of 30 μM to 85 μM.
E66 is the composition of E64 or E65 wherein the particle size distribution of the methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1 is monomodal.
E67 is a co-crystal comprising Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate and an organic carboxylic acid selected from the group consisting of tartaric acid, maleic acid, succinic acid, fumaric acid, 4-hydroxybenzoic acid and 2,5-dihydroxybenzoic acid.
E68 is the co-crystal of E67 wherein the organic carboxylic acid is tartaric acid.
E69 is the co-crystal of E67 wherein the organic carboxylic acid is maleic acid.
E70 is the co-crystal of E67 wherein the organic carboxylic acid is succinic acid.
E71 is the co-crystal of E67 wherein the organic carboxylic acid is fumaric acid.
E72 is the co-crystal of E67 wherein the organic carboxylic acid is 4-hydroxybenzoic acid.
E73 is the co-crystal of E67 wherein the organic carboxylic acid is 2,5-dihydroxybenzoic acid.
Reaction Schemes INT-1, INT-2 and INT-3 depict the preparation of intermediates and compounds used in the processes of the invention.
Reaction Scheme INT-1 depicts the preparation of intermediates 10-14 and Compound 6′.

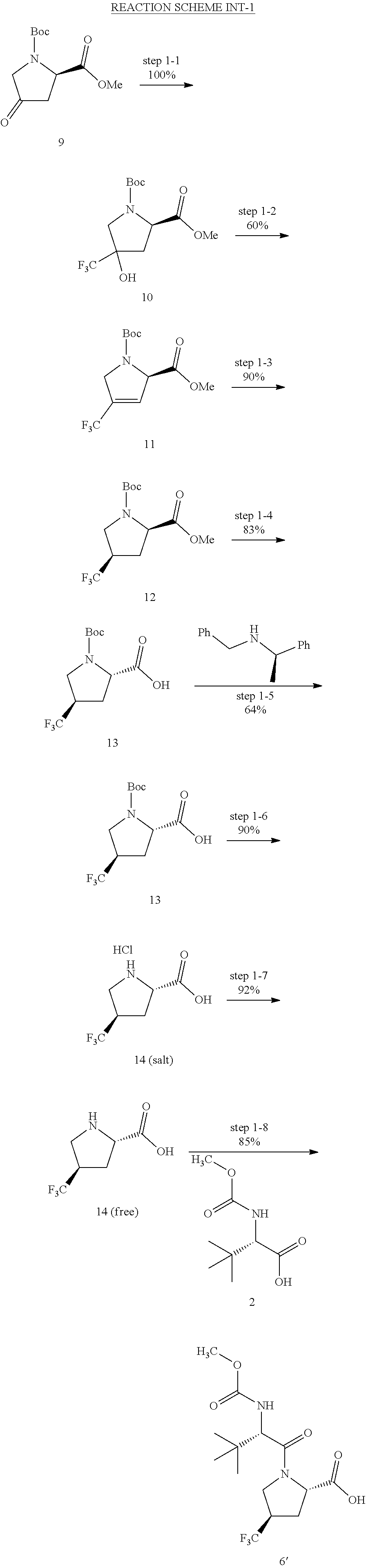

Specifically, Reaction Scheme INT-1 depicts the preparation of (2S,4R)-1-{(2S)-2-[(methoxycarbonyl)amino]-3,3-dimethylbutanoyl}-4-(trifluoromethyl)pyrrolidine-2-carboxylic acid (Compound 6′) starting from 1-tert-Butyl 2-methyl (2R)-4-oxopyrrolidine-1,2-dicarboxylate (Compound 9). In step 1-1 Compound 9 is trifluoromethylated using trimethylsilyl trifluoromethane in the presence of tetrabutyl ammonium fluoride to quantitatively provide 1-tert-Butyl 2-methyl (2R)-4-hydroxy-4-(trifluoromethyl)pyrrolidine-1,2-dicarboxylate (Compound 10). In step 1-2 Compound 10 is subjected to elimination conditions using bis(2-methoxyethyl)aminosulfur trifluoride (BAST) at a pH of 7-8 to provide 1-tert-Butyl 2-methyl (2R)-4-(trifluoromethyl)-2,5-dihydro-1H-pyrrole-1,2-dicarboxylate (Compound 11). In step 1-3 Compound 11 is reduced using hydrogenation with palladium on carbon (Pd/C) as a catalyst to provide 1-tert-Butyl 2-methyl (2R,4R)-4-(trifluoromethyl)pyrrolidine-1,2-dicarboxylate (Compound 12). In step 1-4 Compound 12 is subjected to base catalyzed hydrolysis to provide (2S,4R)-1-(tert-Butoxycarbonyl)-4-(trifluoromethyl)pyrrolidine-2-carboxylic acid (Compound 13) with a diastereomeric ratio of about 75:25 which is then resolved using a chiral salt formation in step 1-5 to provide (2S,4R)-1-(tert-Butoxycarbonyl)-4-(trifluoromethyl) pyrrolidine-2-carboxylic acid (1R)-N-benzyl-1-phenylethanamine (1:1) which is neutralized to provide purified Compound 13 with a greater than 98:2 diastereomeric ratio. In step 1-6 Compound 13 is deprotected under acidic conditions to provide (2S,4R)-4-(Trifluoromethyl)pyrrolidine-2-carboxylic acid hydrochloride (1:1) (Compound 14 HCl salt) which is then neutralized by treatment with base in step 1-7 to provide (2S,4R)-4-(Trifluoromethyl)pyrrolidine-2-carboxylic acid (Compound 14) in its free base form. In step 1-8 Compound 14 (free base) is then coupled with commercially available (2S)-2-[(Methoxycarbonyl)amino]-3,3-dimethylbutanoic acid (Compound 2) under peptide coupling conditions to provide (2S,4R)-1-{(2S)-2-[(methoxycarbonyl)amino]-3,3-dimethylbutanoyl}-4-(trifluoromethyl)pyrrolidine-2-carboxylic acid (Compound 6′). Compound 6′ (free acid) can be coupled with Compound 7 or 7′ in a similar manner as Compound 6 is coupled with compound 7 in Reaction Scheme 1 to provide Compound 8 which can then be dehydrated to provide Compound I or a solvate thereof which can be used to prepare Compound I Form 1 and Compound I Form 1 with MCC.

Reaction Scheme INT-2 depicts the preparation of tert-Butyl (2R,4R)-4-(trifluoromethyl)pyrrolidine-2-carboxylate hydrochloride (1:1) (Compound 3) starting from commercially available Di-tert-butyl (2R)-4-oxopyrrolidine-1,2-dicarboxylate (Compound 15). Compound 15 is reacted with trifluoromethyltrimethylsilane to provide Di-tert-butyl (2R)-4-hydroxy-4-(trifluoromethyl)pyrrolidine-1,2-dicarboxylate (Compound 16) which undergoes an elimination reaction using triflic anhydride/pyridine or alternatively BAST or DAST to provide Di-tert-butyl (2R)-4-(trifluoromethyl)-2,5-dihydro-1H-pyrrole-1,2-dicarboxylate (Compound 17). Compound 17 is then reduced by hydrogenation in the presence of a catalyst such as palladium on carbon (Pd/C) or palladium acetate Pd(OAc)₂ to provide Di-tert-butyl (2R,4R)-4-(trifluoromethyl)pyrrolidine-1,2-dicarboxylate (Compound 18). Compound 18 is then deprotected by treatment under acidic conditions to provide Compound 3. Compound 3 can then be used to prepare Compound I Form 1 as described in Reaction Scheme 1 hereinabove.

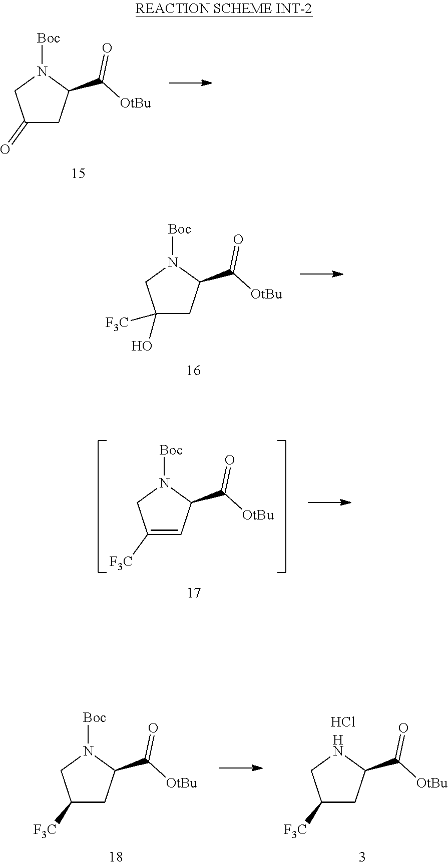

The following are abbreviations that may be used in the specification: ACN is acetonitrile; AcOH is acetic acid; aq. is aqueous; BAST is bis(2-methoxyethyl)aminosulfur trifluoride, °C is degrees Celsius; cm is centimeter; CPME is cyclopentyl methyl ether or methoxycyclopentane; d is doublet; dd is doublet of doublets; ddd is doublet of doublet of doublets; Da is Daltons; DAST is (diethylamino)sulfur trifluoride, DCM is dischloromethane; DMSO is dimethyl sulfoxide; d.r. is diastereomeric ratio; dt is doublet of triplet; EDCI hydrochloride is 1-(3-dimethylamino propyl)-3-ethyl-carbodiimide hydrochloride; ESI is electrospray ionization; EtOAc is ethyl acetate; eq. is equivalent; g is gram; h is hour; HCl is hydrochloric acid or hydrochloride; H₂O is water; H₃PO₄ is phosphoric acid; HRMS is high resolution mass spectroscopy; Hz is Hertz; IPA is isopropyl alcohol; IPAc is isopropyl acetate; K is kelvins; K₂CO₃ is potassium carbonate, kg is kilogram; kHz is kilohertz; KOt-Bu is potassium tert-butoxide; L is liter; m is multiplet; MCC is microcrystalline cellulose; MeCN is acetonitrile; MEK is methyl ethyl ketone; MeCOH is methanol; 2-MeTHF is 2-methyl tetrahydrofuran; MHz is megahertz; min is minute; mL is milliliter; m is micrometer; mmol is millimole; mol is mole; MPa is megapascals; MTBE is methyl tert-butyl ether; NaHCO₃ is sodium bicarbonate; NaOH is sodium hydrochloride; NaOMe is sodium methoxide; NMI is N-methylimidazole; NMM is N-methyl morpholine; NMR is nuclear magnetic resonance; om is overlapped multiplet; Pd/C is palladium on carbon; ppm is parts per million; PXRD is powder x-ray diffraction; q is quartet; s is singlet; Ra Ni is Raney Nickel; ssNMR is solid state nuclear magnetic resonance, t is triplet; TBAF is tetrabutyl ammonium fluoride; TFAA is trifluoroacetic anhydride; THF is tetrahydrofuran; TMSCF₃ is trifluoromethyltrimethylsilane; v/v is volume/volume and w % is weight percent.

### General Experimental Procedures

### Powder X-Ray Diffraction:

The powder X-ray diffraction analysis was conducted using a Bruker AXS D4 Endeavor diffractometer equipped with a Cu radiation source. The divergence slit was set at 0.6 mm while the secondary optics used variable slits. Diffracted radiation was detected by a PSD-Lynx Eye detector. The X-ray tube voltage and amperage were set to 40 kV and 40 mA respectively. Data was collected in the Theta-2Theta goniometer at the Cu wavelength from 3.0 to 40.0 degrees 2-Theta using a step size of 0.020 degrees and a step time of 0.3 second. Samples were prepared by placing them in a silicon low background sample holder and rotated during collection.

The powder X-ray diffraction analysis was conducted using a Bruker AXS D8 Advance diffractometer equipped with a Cu radiation source. Diffracted radiation was detected by a LYNXEYE_EX detector with motorized slits. Both primary and secondary equipped with 2.5 soller slits. The X-ray tube voltage and amperage were set at 40 kV and 40 mA respectively. Data was collected in the Theta-Theta goniometer in a locked couple scan at Cu K-alpha (average) wavelength from 3.0 to 40.0 degrees 2-Theta with an increment of 0.02 degrees, using a scan speed of 0.5 seconds per step. Samples were prepared by placement in a silicon low background sample holder.

Data were collected with both instruments using Bruker DIFFRAC Plus software and analysis was performed by EVA diffract plus software. The PXRD data file was not processed prior to peak searching. Using the peak search algorithm in the EVA software, peaks selected with a threshold value of 1 were used to make preliminary peak assignments. To ensure validity, adjustments were manually made; the output of automated assignments was visually checked, and peak positions were adjusted to the peak maximum. Peaks with relative intensity of 3% were generally chosen. Typically, the peaks which were not resolved or were consistent with noise were not selected. A typical error associated with the peak position from PXRD stated in USP up to +/−0.2° 2-Theta (USP-941)

¹³C and ¹⁹F Solid State Nuclear Magnetic Resonance (ssNMR) Methods
Solid-State Nuclear Magnetic Resonance (ssNMR):

Solid-state NMR (ssNMR) analysis was conducted on a CPMAS probe positioned into a Bruker-BioSpin Avance III 600 MHz (¹H frequency) NMR spectrometer. Material was packed into a ZrO₂ rotor. A magic angle spinning rate of 15 kHz was used. Spectra were collected at ambient temperature (probe temperature of 25 °C.)

¹³C ssNMR spectra were collected using a proton decoupled cross-polarization magic angle spinning (CPMAS) experiment. A phase modulated proton decoupling field of 80-100 kHz was applied during spectral acquisition. The cross-polarization contact time was set to 2 ms and the recycle delay to 3.5 seconds for Form 1. The number of scans was adjusted to obtain an adequate signal to noise ratio. The ¹³C chemical shift scale was referenced using a ¹³C CPMAS experiment on an external standard of crystalline adamantane, setting its up-field resonance to 29.5 ppm.

¹⁹F ssNMR spectra were collected using a proton decoupled magic angle spinning (MAS) experiment. A phase modulated proton decoupling field of 80-100 kHz was applied during spectral acquisition. Spectra were collected with a recycle delay of 3.5 seconds for Form 1. The number of scans was adjusted to obtain an adequate signal to noise ratio. The ¹⁹F chemical shift scale was referenced using a ¹⁹F MAS experiment on an external standard of trifluoroacetic acid (50%/50% v/v in H₂O), setting its resonance to −76.54 ppm.

Automatic peak picking was performed using Bruker-BioSpin TopSpin version 3.6 software. Generally, a threshold value of 5% relative intensity was used for preliminary peak selection. The output of the automated peak picking was visually checked to ensure validity and adjustments were manually made if necessary. Although specific solid-state NMR peak values are reported herein there does exist a range for these peak values due to differences in instruments, samples, and sample preparation. This is common practice in the art of solid-state NMR because of the variation inherent in peak positions. A typical variability for the chemical shift x-axis value is on the order of plus or minus 0.2 ppm for a crystalline solid and plus or minus 0.5 ppm for an amorphous solid. The solid-state NMR peak heights reported herein are relative intensities. Solid-state NMR intensities can vary depending on the actual setup of the experimental parameters and the thermal history of the sample.

### Particle Size Assessment

Particle sizes for the recrystallized materials were assessed using laser diffraction methods. Laser diffraction is recognized by standards and guidance agencies including ISO and ASTM and is widely used to determine particle size distributions. In conducting the assessment, the sample is passed through a laser beam which results in laser light scattered at a range of angles. Detectors placed at fixed angles measure the intensity of light scattered at that position. A mathematical model (Mie or Fraunhoffer Theory) is then applied to generate a particle size distribution.

The particle size was analyzed using the laser diffraction (or small angle light scattering) technique by dispersing the dry sample powder with compressed air. Specifically, the particle size distribution was analyzed using the Sympatec HELOS RODOS system equipped with a Vibri dry powder feeder. The powder sample was dispersed with a dispersion pressure of 0.5 bar. In some instances, an Aspiros micro-dosing device was used, and the powder sample was dispersed with a dispersion pressure of 0.2 bar. A suitable lens was selected to cover the particle size range of each sample.

The surface area of particles can be determined by methods known in the art such as gas absorption methods (BET) initially described by S. Brunauer, P. H. Emmet, E. Teller Adsorption of gases in multimolecular layers J. Am. Chem. Soc., 60 (1938), pp. 309-319.

### Preparation of Intermediates

### Preparation of 1-tert-Butyl 2-methyl (2R)-4-hydroxy-4-(trifluoromethyl)pyrrolidine-1,2-dicarboxylate (Compound 10) (Step 1-1)

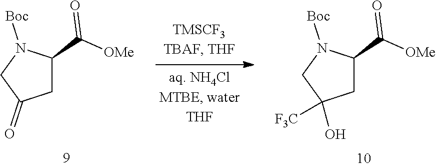 [SMILES: COC(=O)[C@H]1CC(=O)CN1C(=O)OC(C)(C)C ; COC(=O)[C@H]1CC(O)(C(F)(F)F)CN1C(=O)OC(C)(C)C]

Charge 1-tert-Butyl 2-methyl (2R)-4-oxopyrrolidine-1,2-dicarboxylate, Compound 9 into reaction vessel followed by addition of tetrahydrofuran (THF, 7 L/kg of Compound 9) then addition of trimethylsilyltrifluoromethane (TMSCF₃ 1.2 eq.). Adjust temperature to between −15 to −10 °C. Add tetrabutylammonium fluoride (TBAF 1350 mL, 0.14 eq.) into the reaction vessel and adjust temperature to between −5 to −10 °C. Stir the resulting mixture for 16 h at −5 to −10 °C. Add 10% aqueous ammonium chloride (NH₄Cl, 0.5 L/kg of Compound 9) into the reaction vessel then concentrate at 40 °C/−0.085 MPa to remove THF. Add MTBE (4 L/kg of Compound 9) followed by addition of 2% aqueous citric acid (4 L/kg of Compound 9) into the reaction vessel and stir for 10 min. at 20~30 °C. Separate and collect the organic phase and wash the organic phase with water (0.4 L/kg of Compound 9). Concentrate to dry the organic phase and switch with THF until water content by KF<0.5 wt %. Obtain 1-tert-Butyl 2-methyl (2R)-4-hydroxy-4-(trifluoromethyl)pyrrolidine-1,2-dicarboxylate, Compound 10 which is used to next step directly. ¹H NMR (400 MHz, DMSO-d₆, 298K): δ 6.57 (s, 1H), 4.50-4.43 (m, 1H), 3.69-3.65 (m, 3H), 3.61-3.57 (m, 1H), 3.53-3.50 (m, 1H), 2.64-2.49 (m, 1H), 2.14-2.09 (m, 1H), 1.45-1.30 (m, 9H). ¹⁹F NMR (376 MHz, DMSO-d₆, 298K): δ −79.88. ¹³C NMR (101 MHz, DMSO-d₆, 298K): δ 172.5-171.6 (m), 153.9-153.2 (m), 130.0-121.4 (m), 80.3-79.9 (m), 78.8-76.9 (m), 58.1-57.8 (m), 53.6-53.3 (m), 52.6-52.3 (m), 37.4-36.6 (m), 28.3-28.2 (m, 3C). Reference: Angew. Chem. Int. Ed. 2002, 41, 1600-1602.

### Preparation of 1-tert-Butyl 2-methyl (2R)-4-(trifluoromethyl)-2,5-dihydro-1H-pyrrole-1,2-dicarboxylate (Compound 11) (Step 1-2)

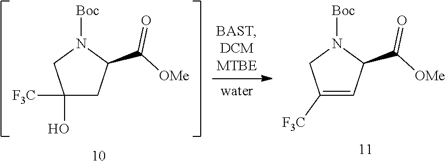

Charge 1-tert-Butyl 2-methyl (2R)-4-hydroxy-4-(trifluoromethyl)pyrrolidine-1,2-dicarboxylate, Compound 10 into an appropriate reaction vessel then add dichloromethane (DCM 5 L/kg of Compound 10). Adjust temperature to −30 to −20 °C then add bis(2-methoxyethyl)aminosulfur trifluoride (BAST, 1.4 eq.) slowly into the reaction vessel at −30 °C to −20 °C over a period of 0.5 h to 3 h. Adjust temperature to 20-30 °C and stir the reaction mixture for 12 h at 20~30 °C then adjust temperature to 0~10 °C and add 7% aqueous sodium bicarbonate (NaHCO₃, 16 L/kg of Compound 10) to the reaction mixture to adjust the pH to 7-8. Stir the reaction mixture for 0.5 h at 20~30 °C. Separate the layers and concentrate the organic layer to remove DCM. Add MTBE (5 L/kg of Compound 10) into the reaction vessel followed by addition of water (2 L/kg of Compound 10). Separate the layers and wash the organic layer with water (2 L/kg of Compound 10). Concentrate the organic layer to dry. Filter crude through 5 kg pad of silica gel and concentrate the resulting solution to dryness. Obtained 3000 g oil 1-(tert-butyl) 2-methyl (R)-4-(trifluoromethyl)-2,5-dihydro-1H-pyrrole-1,2-dicarboxylate, Compound 11 (Isolated yield of 2 steps: 60%). ¹H NMR (400 MHz, DMSO-d₆, 298K): δ 6.72-6.68 (m, 1H), 5.19-5.15 (m, 1H), 4.34-4.30 (m, 2H), 3.72-3.69 (m, 3H), 1.42-1.36 (m, 9H). ¹⁹F NMR (376 MHz, DMSO-d₆, 298K): δ −64.0. ¹³C NMR (101 MHz, DMSO-d₆, 298K): δ 169.5-169.1 (m), 153.1-152.6 (m), 131.3-130.0 (m, 2C), 125.5-117.3 (m), 801.6-80.5 (m), 66.9-66.7 (m), 53.0-52.9 (m), 51.0-50.9 (m), 28.3-28.1 (m, 3C). Reference: Angew. Chem. Int. Ed. 2002, 41, 1600-1602

### Preparation of 1-tert-Butyl 2-methyl (2R,4R)-4-(trifluoromethyl)pyrrolidine-1,2-dicarboxylate (Compound 12) (Step 1-3)

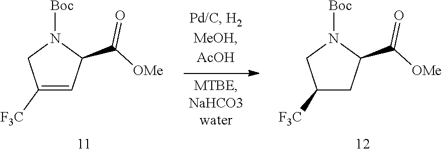 [SMILES: COC(=O)[C@H]1C=C(C(F)(F)F)CN1C(=O)OC(C)(C)C ; COC(=O)[C@H]1C[C@@H](C(F)(F)F)CN1C(=O)OC(C)(C)C]

Charge 1-(tert-butyl) 2-methyl (R)-4-(trifluoromethyl)-2,5-dihydro-1H-pyrrole-1,2-dicarboxylate, Compound 11 (700.00 g) into a reaction vessel followed by addition of methanol (MeOH 10 L/kg of Compound 11). Add palladium on carbon (Pd/C (5 wt %), 0.3 wt % based on Compound 11) into the reaction vessel then add acetic acid (AcOH 0.5 eq.). Purge the reaction vessel with Argon under 0.4 MPa three times then with hydrogen (H₂) under 0.4 MPa three times. Adjust the pressure of the reaction vessel to 45 psi under H₂ flow. Stir the reaction mixture for 16 h at 25-35 °C. Take sample for analysis of reaction completion. Filter the suspension and transfer the methanol liquor. Concentrate at 40 °C-0.085 MPa until no fraction was observed. Obtained an oil which was charged into a second reaction vessel. MTBE (2 L/kg) was added into the second reaction vessel. The resulting mixture was washed with saturated NaHCO₃ solution (3 L/kg) then washed with water (2 L/kg). The organic phase was then concentrated at 40 °C/−0.085 MPa until no distillate fraction was observed. Obtained 1-(tert-butyl) 2-methyl (2R,4R)-4-(trifluoromethyl)pyrrolidine-1,2-dicarboxylate, Compound 12 (Isolated yield 90%). ¹H NMR (400 MHz, DMSO-d₆, 298K): δ 4.37-4.30 (m, 1H), 3.82-3.71 (m, 1H), 3.68-3.62 (m, 3H), 3.43-3.21 (m, 2H), 2.65-2.56 (m, 1H), 1.95-1.79 (m, 1H), 1.43-1.26 (m, 9H). ¹⁹F NMR (376 MHz, DMSO-d₆, 298K): δ −69.5. ¹³C NMR (101 MHz, DMSO-d₆, 298K): δ 172.7-172.2 (m), 153.5-153.0 (m), 131.1-122.8 (m), 80.2-80.1 (m), 58.7-58.4 (m), 52.5-52.4 (m), 41.3-40.7 (m), 30.0-29.1 (m), 28.4-28.2 (m, 3C).

### Preparation of (2S,4R)-1-(tert-butoxycarbonyl)-4-(trifluoromethyl)pyrrolidine-2-carboxylic acid (Compound 13) (with d.r. ~75:25) (Step 1-4)

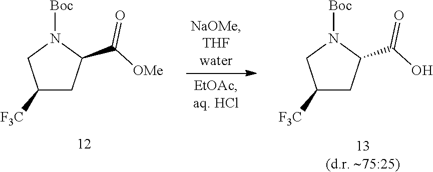 [SMILES: COC(=O)[C@H]1C[C@@H](C(F)(F)F)CN1C(=O)OC(C)(C)C ; CC(C)(C)OC(=O)N1C[C@H](C(F)(F)F)C[C@H]1C(=O)O]

Charge 1-tert-butyl 2-methyl (2R,4R)-4-(trifluoromethyl)pyrrolidine-1,2-dicarboxylate, Compound 12 (100 g) into a reaction vessel followed by addition of tetrahydrofuran (THF 10 L/kg of Compound 12) and adjust the temperature to 25-30 °C. Add sodium methoxide (NaOMe (30% in MeCOH), 2.0 eq) into the reaction vessel at 25-30 °C. Stir the reaction mixture for 2 h at 25-30 °C then add water (5 L/kg Compound 12) and stir for 2 h at 25-30 °C. The reaction mixture is then neutralized with HCl (3 M) to pH 7-8 at 5-10 °C. Remove MeCOH under vacuum and extract the resulting mixture with ethyl acetate (2× with 1 L EtOAc/kg of Compound 12). Acidified the aqueous layer with HCl (1 M) to pH 3~4 and extracted this with EtOAc (3× with 1 L EtOAc/kg of Compound 12). Combined organic layers were concentrated to obtain (2S,4R)-1-(tert-butoxycarbonyl)-4-(trifluoromethyl)pyrrolidine-2-carboxylic acid, Compound 13 in mixture with other diastereomer (isolated yield: 83%; typical d.r. ~75:25).

### Resolution of (2S,4R)-1-(tert-butoxycarbonyl)-4-(trifluoromethyl)pyrrolidine-2-carboxylic acid (Compound 13) (from d.r. ~75:25 to d.r. >98:2) (Step 1-5—chiral resolution step)

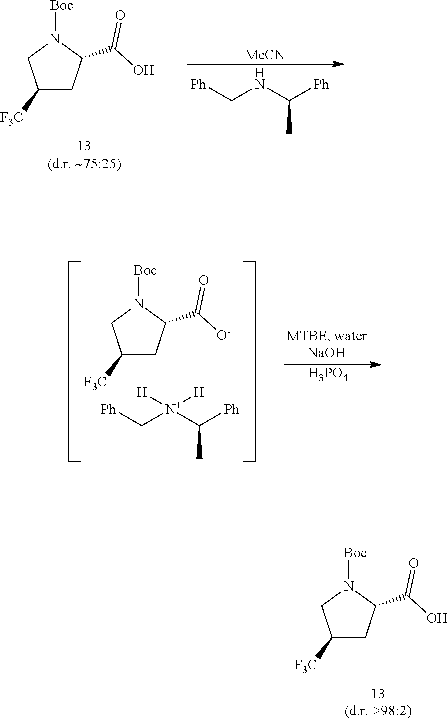

Procedure: Add (2S,4R)-1-(tert-butoxycarbonyl)-4-(trifluoromethyl)pyrrolidine-2-carboxylic acid, Compound 13 (d.r. ~75:25 mixture) and acetonitrile, MeCN (17 L/kg Compound 13) into reaction vessel and adjust temperature to 25-30 °C. To this add a solution of (1R)-N-benzyl-1-phenyl-ethanamine (1.3 eq. based on pure Compound 13) in MeCN (3 L/kg of (1R)-N-benzyl-1-phenyl-ethanamine) at 25-30 °C over a period of 5 h. Stir the resulting mixture for 16 h at 25-30 °C. Filter the mixture and rinse the wet filter cake with MeCN (2 L/kg of Compound 13). Dry the filter cake under vacuum at 35-45 °C. Obtained the (2S,4R)-1-(tert-butoxycarbonyl)-4-(trifluoromethyl)pyrrolidine-2-carboxylic acid (1R)-N-benzyl-1-phenyl-ethanamine salt (isolated yield: 76%; typical chiral purity: >99.9%). Add this salt and water (3 L/kg of the salt) into a reaction vessel and adjust temperature to 25-30 °C. To this add aqueous sodium hydroxide (2 eq. NaOH in 1 L H₂O/kg of the salt) at 25-30 °C. Stir the resulting reaction mixture for 2 h at 25-30 °C. Add MTBE (5 L/kg) into the reaction vessel and stir for 0.5 h then separate the layers. Extract the aqueous layer with MTBE (3 L/kg) twice. Adjust the temperature to 0-10 °C then adjust pH of the mixture to 3-4 with 1 μM H₃PO₄ at 0-10 °C. Stir the mixture for 3 h at 0-10 °C. Filter and rinse the filter cake with water (1 L/kg). Dry the filter cake under vacuum at 35-45 °C. Obtained (2S,4R)-1-(tert-butoxycarbonyl)-4-(trifluoromethyl)pyrrolidine-2-carboxylic acid (Compound 13) (isolated yield: 85%). Product Characterization of (2S,4R)-1-(tert-butoxycarbonyl)-4-(trifluoromethyl) pyrrolidine-2-carboxylic acid: The sample contains a minor rotamer making up approximately 40% of the sample.

¹H NMR (600 MHz, DMSO-d₆, 298K): δ 12.81 (s, 1H), 4.26 (dd, J=9.28, 3.10 Hz, minor), 4.22 (dd, J=9.09, 3.63 Hz, 1H), 3.62 (m, 1H), 3.42 (d, J=11.26 Hz, 1H), 3.26 (m, 1H), 2.42-2.37 (m, 1H) 2.39-2.32 (m, minor), 2.21-2.15 (m, 1H), 2.19-2.13 (m, minor) 1.40 (s, minor), 1.35 (s, 9H). ¹⁹F NMR (376 MHz, DMSO-d₆, 298K): δ −70.2 ¹³C NMR (151 MHz, DMSO-d₆, 298K): δ 173.3, 172.9 (minor), 153.0 (minor), 152.8, 127.0 (1JCF=277.5 Hz), 126.9 (1JCF=277.5 Hz, minor), 79.5 (minor) 79.4, 58.1, 57.9 (minor), 45.3 (minor), 45.2, 40.3 (2JCF=27.2 Hz, minor), 39.7 (2JCF=27.2 Hz), #29.2, 28.4 (minor), 27.9 (minor), 27.8. #=Due to overlap with the solvent, the coupling constant could not accurately be determined. It was calculated as equivalent to the minor rotamer. HRMS: (ESI+) Calcd for C₁₁H₁₇F₃NO₄⁺: 284.1104, Found: 284.1102 (mass deviation −0.8 ppm).

### Preparation of (2S,4R)-4-(Trifluoromethyl)pyrrolidine-2-carboxylic acid hydrochloride (1:1), (Compound 14, HCl salt) (Step 1-6) in Ethyl Acetate or Dioxane

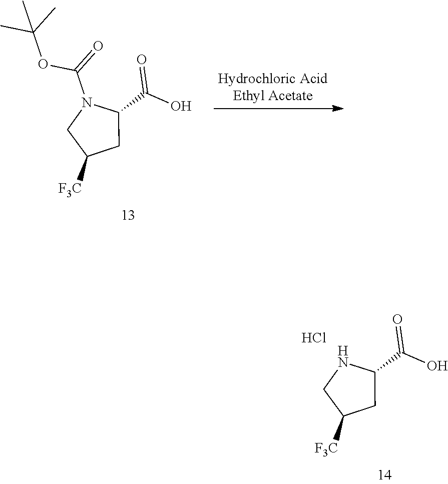 [SMILES: CC(C)(C)OC(=O)N1C[C@H](C(F)(F)F)C[C@H]1C(=O)O ; Cl ; O=C(O)[C@@H]1C[C@@H](C(F)(F)F)CN1]

### Procedure In Ethyl Acetate:

(2S,4R)-1-(tert-butoxycarbonyl)-4-(trifluoromethyl)pyrrolidine-2-carboxylic acid, Compound 13 (20 g, 71 mmol, 1.0 equivalents) and hydrochloric acid (1 Mol/L) in ethyl acetate (309 mL, 309 mmol, 4.3 equivalents) are combined and stirred at 20 °C. The mixture is warmed to 50 °C over not less than 1 h and the mixture is stirred for 20 h. A sample is analyzed for reaction completion by UPLC (target not more than 1% Compound 13). The mixture is cooled to 10 °C over not less than 1 h and stirred. The solids are collected by filtration, rinsed with ethyl acetate (100 mL, 5 mL/g of Compound 13) and twice with ethyl acetate (50 mL, 2.5 mL/g of Compound 13) and dried at 45 °C in a vacuum oven to provide (2S,4R)-4-(Trifluoromethyl)pyrrolidine-2-carboxylic acid hydrochloride (1:1).

### Procedure In Dioxane:

(2S,4R)-1-(tert-butoxycarbonyl)-4-(trifluoromethyl)pyrrolidine-2-carboxylic acid, Compound 13 (141 g, 498 mmol, 1.0 equivalents) and dichloromethane (850 mL, 6 mL/g of Compound 13) are combined and stirred at 25 °C. Hydrochloric Acid (4 Mol/L) in 1,4-dioxane (500 mL, 2000 mmol, 4.0 equivalents) is added over not less than 1 h. The mixture is then stirred for 22 h. A sample is analyzed for reaction completion by UPLC (target not more than 1% Compound 13). The slurry is transferred to a separate vessel and is concentrated to remove volatiles. Ethyl acetate (700 mL, 5 mL/g of Compound 13) is charged and stirred for not less than 15 minutes. The solids are collected by filtration. The wet filter cake is washed three times with ethyl acetate (300 mL, 2.1 mL/g of Compound 13). The solids are dried at 40 °C in a vacuum oven to provide (2S,4R)-4-(Trifluoromethyl)pyrrolidine-2-carboxylic acid hydrochloride (1:1).

¹H NMR (400 MHz, DMSO-d₆, 298K): δ 10.52 (br s, 2H), 4.41 (t, 1H), 3.64 (dd, 1H), 3.47 (dtd, 1H), 3.21 (dd, 1H), 2.46-2.30 (m, 2H). ¹⁹F NMR (376 MHz, DMSO-d₆, 298K): δ −69.61 (d, J=9.4 Hz). ¹³C NMR (101 MHz, DMSO-d₆, 298K): δ 168.87, 126.52 (q, J=277.8 Hz), 58.57, 43.70 (d, J=3.1 Hz), 39.84 (partially overlaps with DMSO signal), 27.52 (d, J=2.6 Hz). HRMS: (ESI+) Calcd for C₆H₉F₃NO₂⁺: 184.0580, Found: 184.0579 (mass deviation −0.5 ppm). PXRD was determined for different batches of Compound 14 HCl salt and two different PXRD patterns were observed (See Figures X and Y) in which the peaks (each ±0.2 °2-Theta) in the following tables were observed.

### PXRD Peaks For Compound 14 HCl Salt—Pattern 1

| Angle (°2θ ± | Rel. |
|---|---|
| 0.2 °2θ) | Intensity |
| 7.5 | 1 |
| 9.7 | 1 |
| 14.9 | 1 |
| 15.4 | 4 |
| 15.8 | 1 |
| 16.9 | 1 |
| 17.6 | 1 |
| 18.7 | 1 |
| 19.4 | 2 |
| 21.5 | 5 |
| 21.9 | 3 |
| 22.2 | 2 |
| 22.5 | 100 |
| 22.8 | 2 |
| 23.3 | 1 |
| 23.6 | 5 |
| 24.4 | 3 |
| 24.8 | 2 |
| 26.9 | 1 |
| 27.2 | 1 |
| 27.7 | 2 |
| 28.6 | 1 |
| 28.8 | 1 |
| 30.2 | 19 |
| 31.9 | 2 |
| 32.5 | 3 |
| 33.2 | 1 |
| 33.7 | 1 |
| 35.9 | 2 |
| 37.0 | 1 |
| 38.0 | 10 |
| 39.3 | 1 |

PXRD Peaks from Compound 14 HCl Salt—Pattern 2

| Angle (°2θ ± | Rel. |
|---|---|
| 0.2 °2θ) | Intensity |
| 7.6 | 81.6 |
| 14.8 | 5.4 |
| 15.3 | 71.2 |
| 15.6 | 20.1 |
| 16.3 | 27.9 |
| 17.9 | 34.9 |
| 18.4 | 27.8 |
| 19.0 | 35.0 |
| 19.5 | 1.9 |
| 20.1 | 100.0 |
| 21.6 | 4.8 |
| 22.1 | 1.5 |
| 22.3 | 0.9 |
| 22.8 | 91.4 |
| 23.4 | 46.6 |
| 23.7 | 3.3 |
| 24.6 | 8.8 |
| 24.9 | 0.8 |
| 25.8 | 45.0 |
| 26.7 | 32.1 |
| 27.1 | 21.5 |
| 27.4 | 18.0 |
| 27.8 | 1.5 |
| 28.5 | 9.6 |
| 29.7 | 43.3 |
| 30.3 | 34.4 |
| 30.5 | 85.9 |
| 31.4 | 6.8 |
| 32.2 | 41.5 |
| 32.9 | 5.4 |
| 33.5 | 13.4 |
| 34.2 | 36.8 |
| 35.1 | 4.4 |
| 35.9 | 9.3 |
| 36.2 | 4.2 |
| 36.5 | 13.5 |
| 37.1 | 6.3 |
| 38.1 | 0.7 |
| 39.4 | 3.4 |
| 39.8 | 5.2 |

### Preparation of (2S,4R)-4-(Trifluoromethyl)pyrrolidine-2-carboxylic acid (Compound 14, free form) (Step 1-7)

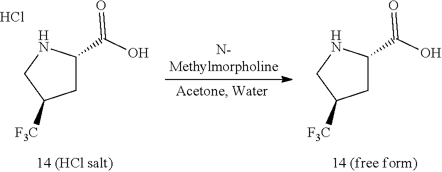 [SMILES: Cl ; O=C(O)[C@@H]1C[C@@H](C(F)(F)F)CN1 ; O=C(O)[C@@H]1C[C@@H](C(F)(F)F)CN1]

Add (2S,4R)-4-(trifluoromethyl)pyrrolidine-2-carboxylic acid hydrochloride, Compound 14 (50.00 g, 223 mmol, 1 eq.) into a reaction vessel followed by addition of acetone (150 mL, 4 L/kg) and water (50 mL, 1 L/kg). Add 4-Methylmorpholine (24.7 mL, 224 mmol, 1.00 eq.) slowly over 1 to 2 h. Note: Slurry gets thick and can stagnate. Stir mixture for 2 h then filter slurry. Washed filter cake with 4:1 acetone:water mixture (125 mL, 2.5 L/kg) twice. Dry solid in a vacuum oven at 70 °C overnight (16 h) to afford desired product: (2S,4R)-4-(trifluoromethyl)pyrrolidine-2-carboxylic acid also known as (2S,4R)-4-(trifluoromethyl) pyrrolidin-1-ium-2-carboxylate; 93-95% yield, 96.7-98.83% potency.

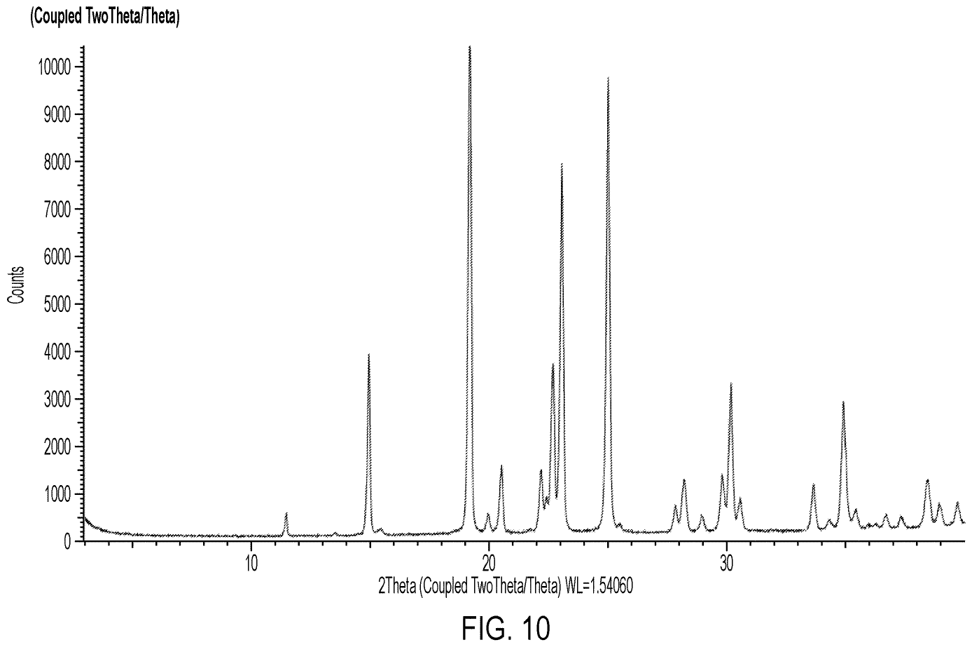

¹H NMR (600 MHz, D20, 298K): δ 4.27 (dd, J=8.57, 7.01 Hz, 1H), 3.73 (dd, J=12.93, 8.71 Hz, 1H), 3.52 (dd, J=12.93, 6.19 Hz, 1H), 3.35 (m, 1H), 2.53 (ddd, J=14.41, 8.57, 6.10 Hz, 1H), 2.41 (ddd, J=14.40, 8.54, 7.01 Hz, 1H). ¹⁹F NMR (400 MHz, D20, 298K): δ −71.44 (s). ¹³C NMR (151 MHz, D20, 298K): δ 173.0, 126.2 (¹J_{CF}=277.0 Hz), 61.1, 44.5 (³J_{CF}=3.0 Hz), 40.8 (²J_{CF}=29.4 Hz), 28.6 (³J_{CF}=2.5 Hz). HRMS: (ESI+) Calcd for C₆H₉F₃NO₂⁺: 184.0580, Found: 184.0575 (mass deviation −2.7 ppm) PXRD for Compound 14 free acid was determined (See FIG. 10) and the peaks in the following table were observed.

| Angle (°2θ ± | Rel. |
|---|---|
| 0.2 °2θ) | Intensity |
| 11.5 | 4 |
| 13.5 | 1 |
| 15.0 | 36 |
| 15.4 | 1 |
| 19.2 | 100 |
| 20.0 | 4 |
| 20.5 | 13 |
| 21.8 | 1 |
| 22.2 | 12 |
| 22.4 | 7 |
| 22.7 | 33 |
| 23.1 | 77 |
| 23.8 | 0 |
| 24.0 | 0 |
| 24.3 | 0 |
| 25.0 | 94 |
| 25.5 | 2 |
| 27.9 | 6 |
| 28.2 | 10 |
| 29.0 | 4 |
| 29.8 | 12 |
| 30.2 | 31 |
| 30.6 | 7 |
| 34.3 | 2 |
| 34.9 | 27 |
| 35.4 | 4 |
| 36.0 | 1 |
| 36.3 | 1 |
| 36.7 | 3 |
| 37.3 | 3 |
| 37.9 | 0 |
| 38.5 | 9 |
| 39.0 | 4 |
| 39.7 | 5 |

### Preparation of Di-tert-butyl (2R)-4-hydroxy-4-(trifluoromethyl)pyrrolidine-1,2-dicarboxylate (Compound 16)

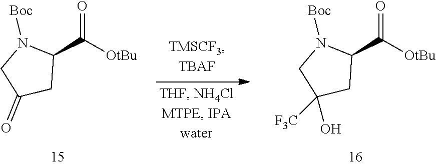 [SMILES: CC(C)(C)OC(=O)[C@H]1CC(=O)CN1C(=O)OC(C)(C)C ; CC(C)(C)OC(=O)[C@H]1CC(O)(C(F)(F)F)CN1C(=O)OC(C)(C)C]

Di-tert-butyl (2R)-4-oxopyrrolidine-1 2-dicarboxylate, Compound 15 (di-tert-butyl (2R)-4-oxopyrrolidine-1 2-dicarboxylate) (40 g, 1.0 equivalents) and tetrahydrofuran (240 mL, 6 L/kg Compound 15) are combined and stirred at 25 °C. Trifluoromethyl trimethylsilane, TMSCF₃ (29.7 mL, 1.5 eq.) added and the resulting solution is cooling down at 0-15 °C (reaction below 0 °C had a better exotherm control). Tetrabutylammonium fluoride, TBAF (9.06 g, 0.26 eq.) was mixed with tetrahydrofuran (40 mL, 1 L/kg Compound 15) and added dropwise over previous reaction mixture (highly exotherm was observed with the first drop (~10-15 °C)). Rinse with tetrahydrofuran (40 mL, 1 L/kg Compound 15), and stirred at 25 °C for 16 hours. The reaction is sampled for analysis (target complete conversion). The reaction is quenched by the addition of aqueous ammonium chloride, NH₄Cl (360 mL of a 10 wt % brine solution, 9.0 L/kg of Compound 15), added methyl tert-butyl ether (200 mL, 5 L/kg Compound 15) and stirring is maintained for 30 minutes. Stirring is stopped and the layers allowed to settle. The organic phase is removed and washed with a second portion of methyl tert-butyl ether (200 mL, 5 L/kg Compound 15). The organic phases are combined and then concentrated by vacuum distillation at 0.3 bar (internal temperature of reaction mixture ~30 °C) up to reached ~5 L/kg. Then isopropanol (400 mL, 10 L/kg Compound 15) was added to the mixture and distillation continue to reach ~5 L/kg reaction volume. A second addition of isopropanol (400 mL, 8 L/kg Compound 15) is added, and the distillation process was repeated following the same protocol, ending the distillation at 5 L/kg. Water (200 mL, 4-5 L/kg Compound 15) was added slowly and stirred at 70 °C for 30 minutes to reach a solution (added 0-1 L/Kg isopropanol if need to reach clear solution). This solution was cooled to 25 °C in 2 hours follow by slow cooling to 0 °C (0.2K/min ramp) and hold at 0 °C for at least 30 minutes. Solids are collected by filtration, rinsed with 2 L/kg of a 1:1 mixture of isopropanol:water twice, and dried in a vacuum oven at 50 °C for 12 hours providing 36.2 g of Di-tert-butyl (2R)-4-hydroxy-4-(trifluoromethyl)pyrrolidine-1,2-dicarboxylate, Compound 16 (73-88% yield). Note: in the ¹H, ¹⁹F and ¹³C NMR spectra, 2 sets of resonances were observed due to the presence of rotamers in solution. ¹H NMR (400 MHz, DMSO-d₆, 298K): δ 6.51 (s, 1H), 4.31 (ddd, J=16.9, 9.5, 2.0 Hz, 1H), 3.50 (dd, J=12.0, 5.7 Hz, 1H), 2.67-2.41 (m, 1H), 2.11-2.03 (m, 1H), 1.45-1.34 (m, 18H). ¹⁹F NMR (376 MHz, DMSO-d₆, 298K): δ −79.61, −79.65. ¹³C NMR (101 MHz, DMSO-d₆, 298K): 5 170.8, 170.5, 153.7, 127.6, 124.7, 81.5, 81.4, 80.2, 80.0, 78.6, 77.9, 77.6, 59.1, 58.9, 54.0, 53.8, 38.1, 37.3, 28.8, 28.6, 28.4, 28.3, 26.3. HRMS: Calcd for C₁₅H₂₅F₃NO₅⁺: 356.1680, Found: 356.1693 (mass deviation 3.79 ppm).

### Preparation of Di-tert-butyl (2R)-4-(trifluoromethyl)-2,5-dihydro-1H-pyrrole-1,2-dicarboxylate (Compound 17)

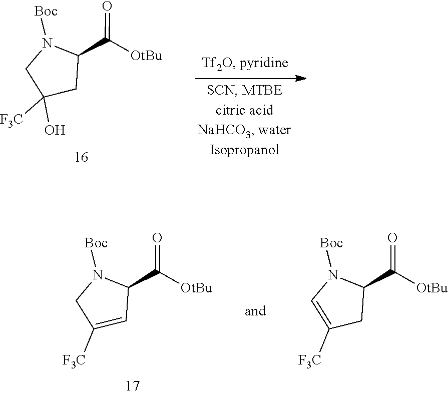 [SMILES: CC(C)(C)OC(=O)[C@H]1CC(O)(C(F)(F)F)CN1C(=O)OC(C)(C)C ; CC(C)(C)OC(=O)[C@H]1C=C(C(F)(F)F)CN1C(=O)OC(C)(C)C ; CC(C)(C)OC(=O)[C@H]1CC(C(F)(F)F)=CN1C(=O)OC(C)(C)C]

Di-tert-butyl (2R)-4-hydroxy-4-(trifluoromethyl)pyrrolidine-1,2-dicarboxylate, Compound 16 (10.6 g, 1.0 equivalents) and acetonitrile (45 mL, 4 L/kg Compound 16) are combined and stirred at 25 °C. Pyridine (12 mL, 5 equivalents) is added, and the resulting solution is heated to 30-40 °C. Triflic anhydride (7.4 mL, 1.5 eq.) is added dropwise (exotherm was observed (~10 °C)), and stirred at 40 °C for 16-20 h. The reaction is sampled for IPC (target complete conversion). The reaction is cooled to 0-5 °C and quenched by the addition of purified water (32 mL, 3.0 L/kg of Compound 16) dropwise, stirring is maintained for 30 minutes, and added methyl tert-butyl ether (85 mL, 8.0 L/kg of Compound 16). Mixture is warmed to 25 °C and stirring is maintained for 30 minutes. Stirring is stopped and the layers allowed to settle. The aqueous phase is removed, and organic phase is washed with an aqueous citric acid (64 mL of a 2 wt % citric acid solution, 6.0 L/kg of Compound 16) follow by another wash with an aqueous sodium bicarbonate (64 mL of a 7 wt % sodium bicarbonate solution, 6.0 L/kg of Compound 16), and two washes with purified water (32 mL, 3.0 L/kg of Compound 16) following the same protocol. Organic phase is concentrated by vacuum distillation at 0.3 bar (internal temperature of reaction mixture ~30-40 °C) up to reached ~2.5 L/kg. Then methanol or isopropanol (50 mL, 5 L/kg Compound 16) was added to the mixture and distillation continue to reach ~2.5 L/kg reaction volume. A second addition of isopropanol (50 mL, 5 L/kg Compound 16) is added, and the distillation process was repeated following the same protocol, ending the distillation at 2.5 L/kg. A sample is analyzed for solvent content with a target of not less than 96% iPrOH vs. ACN, MTBE. If target is not reached repeat distillation cycles until specifications are met. The product is isolated as a mixture of positional isomers (Compound 17, Di-tert-butyl (2R)-4-(trifluoromethyl)-2,5-dihydro-1H-pyrrole-1,2-dicarboxylate with 0.5-20% of Di-tert-butyl (2R)-4-(trifluoromethyl)-2,5-dihydro-1H-pyrrole-1,2-dicarboxylate) in a solution of isopropanol (or methanol). Expected molar yield: 75-90% yield. The solution is used in the following step without further processing.

### Alternative procedure for Preparation of Di-tert-butyl (2R)-4-(trifluoromethyl)-2,5-dihydro-1H-pyrrole-1,2-dicarboxylate (Compound 17) using BAST or DAST

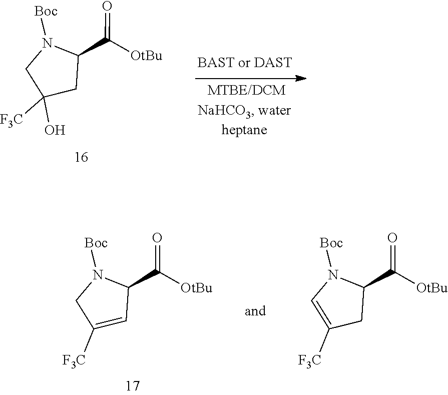 [SMILES: CC(C)(C)OC(=O)[C@H]1CC(O)(C(F)(F)F)CN1C(=O)OC(C)(C)C ; CC(C)(C)OC(=O)[C@H]1C=C(C(F)(F)F)CN1C(=O)OC(C)(C)C ; CC(C)(C)OC(=O)[C@H]1CC(C(F)(F)F)=CN1C(=O)OC(C)(C)C]

Compound 16 (106 g, 1.0 equivalents) and methyl tert-butyl ether (10 L/kg Compound 16) are combined and stirred at (−10)−0 °C. Bis(2-methoxyethyl)aminosulfur trifluoride, BAST (1.7 equivalents) is added slowly into the mixture at (−10)-0 °C, and the resulting solution is warmed to 20-30 °C and stirring for 16-20 hours. The reaction is sampled for complete conversion. The reaction is cooled to 0-10 °C and quenched by the addition of an aqueous sodium bicarbonate (7 wt % sodium bicarbonate solution, ~16.0 L/kg of Compound 16) to adjust pH=7-8. Mixture is warmed to 25 °C and stirring is maintained for 30 minutes. Stirring is stopped and the layers allowed to settle. The aqueous phase is removed, and organic phase is washed with water (4.0 L/kg of Compound 16). Organic phase is concentrated by vacuum distillation up to reached ~1-2 L/kg. Then heptane (10 L/kg Compound 16) was added to the mixture and filtrated through silica gel pad (0.5 g/g) with heptane/MTBE (50:1) as eluent. Filtrate was concentrated to reached ~1-2 L/kg. The product is isolated as a mixture of positional isomers (Di-tert-butyl (2R)-4-(trifluoromethyl)-2,5-dihydro-1H-pyrrole-1,2-dicarboxylate, Compound 17 with 0.5-20% of Di-tert-butyl (2R)-4-(trifluoromethyl)-2,5-dihydro-1H-pyrrole-1,2-dicarboxylate) in a solution of heptane. Expected molar yield: 75-90% yield. The solution is used in the following step without further processing.

Note: The procedure above can be carried out using (diethylamino)sulfur trifluoride, DAST instead of BAST and with MTBE or DCM as solvent.

A sample of solution was concentrated to dryness and purified by column chromatography for characterization purpose using a solvent mixture of heptane:ethyl acetate (80:20) and RediSep Gold® Silica Gel Disposable Flash Columns.

Note: in the ¹H, ¹⁹F and ¹³C NMR spectra, 2 sets of resonances were observed due to the presence of rotamers in solution. A minor positional isomer Di-tert-butyl (2R)-4-(trifluoromethyl)-2,5-dihydro-1H-pyrrole-1,2-dicarboxylate (indicated as minor when possible) is also observed with Compound 17, and both are isolated together. Both isomers are processed together in the following step which provides the same product.

¹H NMR (400 MHz, DMSO-d₆, 298K): δ 7.22 (d, J=31.4 Hz, minor), 6.78-6.51 (m, 1H), 5.13-4.94 (m, 1H), 4.70 (dd, J=12.3, 4.8 Hz, minor), 4.29 (dtq, J=5.8, 4.0, 1.7 Hz, 2H), 2.65 (d, J=17.4 Hz, minor), 1.46-1.36 (m, 18H). ¹⁹F NMR (376 MHz, DMSO-d₆, 298K): δ −61.22 (minor), −63.78, −63.81. ¹³C NMR (101 MHz, DMSO-d₆, 298K): δ 170.1, 168.2, 167.9, 153.4, 153.0, 151.2, 135.0, 131.8, 131.7, 131.7, 131.7, 131.5, 131.5, 131.4, 131.2, 131.2, 130.9, 130.9, 130.5, 130.2, 125.8, 125.2, 123.2, 123.1, 122.6, 120.5, 120.4, 120.0, 117.8, 107.8, 107.7, 107.3, 106.9, 106.6, 106.5, 106.5, 106.2, 106.0, 82.7, 82.5, 82.3, 80.7, 80.6, 68.0, 67.8, 60.0, 51.3, 32.9, 32.0, 31.7, 29.1, 28.7, 28.5, 28.3, 28.3, 28.2, 28.2, 27.7, 22.9, 14.7. HRMS: Calcd for C₁₅H₂₃F₃NO₄⁺: 338.1574, Found: 338.1574 (mass deviation −0.015 ppm)

### Preparation of Di-tert-butyl (2R,4R)-4-(trifluoromethyl)pyrrolidine-1,2-dicarboxylate (Compound 18)

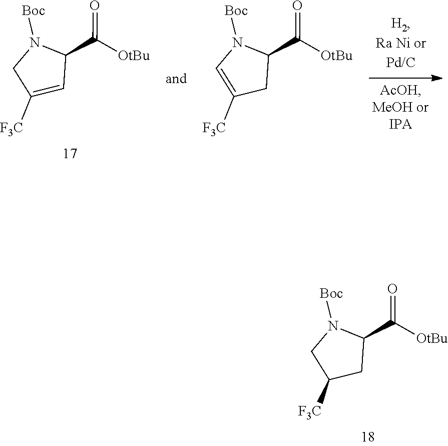 [SMILES: CC(C)(C)OC(=O)[C@H]1C=C(C(F)(F)F)CN1C(=O)OC(C)(C)C ; CC(C)(C)OC(=O)[C@H]1CC(C(F)(F)F)=CN1C(=O)OC(C)(C)C ; CC(C)(C)OC(=O)[C@H]1C[C@@H](C(F)(F)F)CN1C(=O)OC(C)(C)C]

To a solution of Di-tert-butyl (2R)-4-(trifluoromethyl)-2,5-dihydro-1H-pyrrole-1,2-dicarboxylate, Compound 17 with 0-20% of Di-tert-butyl (2R)-4-(trifluoromethyl)-2,5-dihydro-1H-pyrrole-1,2-dicarboxylate (90 g, 1.0 equivalents) in methanol (6-10 L/kg Compound 17+Di-tert-butyl (2R)-4-(trifluoromethyl)-2,5-dihydro-1H-pyrrole-1,2-dicarboxylate) or isopropanol (2-3 L/kg Compound 17+Di-tert-butyl (2R)-4-(trifluoromethyl)-2,5-dihydro-1H-pyrrole-1,2-dicarboxylate), acetic acid (0.3-0.5 eq.) is added. at 20-30 °C. Raney Nickel (0.4-1 w/w) was added, and system was swapped with nitrogen under 0.4 MPa three times, then swapped to hydrogen under 0.4 MPa three times and adjusted to 0.6-0.9 MPa with hydrogen and the resulting slurry is stirring at 25-35 °C for 16-24 hours. (Note: Raney-Nickel can be reduced to 25% wet catalyst (60% w/w cat.+40% water), and pH of catalyst can be adjusted prior to use with acetic acid, so addition of AcOH is not needed). The reaction is sampled for analysis (target complete conversion). Reaction mixture is filter and concentrate to dryness. Methyl tert-butyl ether (5.0 L/kg of Compound 17+Di-tert-butyl (2R)-4-(trifluoromethyl)-2,5-dihydro-1H-pyrrole-1,2-dicarboxylate) is added, solution is stirring at 25 °C and washed with an aqueous sodium bicarbonate (7 wt % sodium bicarbonate solution, 3.0 L/kg of Compound 17+Di-tert-butyl (2R)-4-(trifluoromethyl)-2,5-dihydro-1H-pyrrole-1,2-dicarboxylate). Stirring is maintained for 30 minutes. Stirring is stopped and the layers allowed to settle. The aqueous phase is removed, and organic phase is washed with water (2.0 L/kg of Compound 17+Di-tert-butyl (2R)-4-(trifluoromethyl)-2,5-dihydro-1H-pyrrole-1,2-dicarboxylate). Further distillation if needed to reach water content of not more than 0.1%. Di-tert-butyl (2R,4R)-4-(trifluoromethyl)pyrrolidine-1,2-dicarboxylate, Compound 18 was obtained in 93% isolated yield.

Crystallization conditions: When isopropanol is used as solvent for this reaction, after catalyst filtration, solution is heated at 50 °C and water (2.0 L/kg of Compound 17+Di-tert-butyl (2R)-4-(trifluoromethyl)-2,5-dihydro-1H-pyrrole-1,2-dicarboxylate) is added slowly at 50 °C. Mixture is cooled up to 5-10 °C to initiate crystallization (seeding at −30 °C if needed). Compound is isolated by filtration.

Characterization of Di-tert-butyl (2R,4R)-4-(trifluoromethyl)pyrrolidine-1,2-dicarboxylate, Compound 18, Note: The sample contains a minor rotamer due to the N1-C15 amide bond, making up approximately 11% of the sample.

¹H NMR (600 MHz, DMSO-d₆, 298K): δ 4.21-4.17 (om, 1H), 3.77-3.73 (om, 1H), 3.40-3.30 (m, 1H), 3.29-3.21 (om, 1H), 2.64 (dt, J=13.29, 9.00 Hz, 1H), 2.59 (dt, J=13.21, 9.00 Hz, minor), 1.88-1.81 (m, 1H), 1.42 (s, 9H), 1.41 (s, minor), 1.39 (s, minor), 1.36 (s, 9H). ¹⁹F NMR (376 MHz, DMSO-d₆, 298K): δ −69.65 (d, J=0.02 Hz), −69.70 (d, J=0.02 Hz, minor). ¹³C NMR (151 MHz, DMSO-d₆, 298K): δ 170.6, 170.2 (minor), 152.9 (minor), 152.7, 126.6 (¹J_{CF}=277.1 Hz), 80.9, 80.7 (minor), 79.5 (minor), 79.3, 58.7 (minor), 58.4, 45.4 (³J_{CF}=3.00 Hz), 40.1 (²J_{CF}=27.9 Hz, minor), 39.3 (overlapped with solvent), 29.3, 28.3 (minor), 27.9 (minor), 27.7, 27.4. om=overlapped multiplet. HRMS: (ESI+) Calcd for C₁₅H₂₅F₃NO₄⁺: 340.1730, Found: 340.1733 (mass deviation 0.79 ppm). Reference: J. Org. Chem. 2003, 68, 9, 3614-3617.

### Preparation of tert-Butyl (2R,4R)-4-(trifluoromethyl)pyrrolidine-2-carboxylate hydrochloride (1:1) (Compound 3)

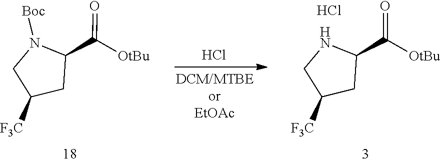 [SMILES: CC(C)(C)OC(=O)[C@H]1C[C@@H](C(F)(F)F)CN1C(=O)OC(C)(C)C ; Cl ; CC(C)(C)OC(=O)[C@H]1C[C@@H](C(F)(F)F)CN1 ; Cl]

Add di-tert-butyl (2R,4R)-4-(trifluoromethyl)pyrrolidine-1,2-dicarboxylate, Compound 18 (10.00 g, 29.46 mmol, 1.00 eq.) to a reaction vessel then add dichloromethane (50 mL, 5 L/kg of Compound 18) to the reaction vessel. Add hydrochloric acid (30 mL, 120 mmol, 4.073 eq.) in 1,4-dioxane slowly to the mixture. Stir the reaction mixture for 3 hours. Concentrate mixture to remove volatiles. Add methyl tert-butyl ether (MTBE, 50 mL, 5 L/kg of Compound 18) to the mixture then concentrate mixture to remove volatiles. Add methyl tert-butyl ether (50 mL, L/kg of Compound 18) and stir for 30 min to 1 h. Filter the resulting slurry. Washed filter cake with 20 mL (2 L/kg of Compound 18) of methyl tert-butyl ether twice. Dry solid in a vacuum oven at 50 °C overnight (16 h) to afford the desired product tert-Butyl (2R,4R)-4-(trifluoromethyl)pyrrolidine-2-carboxylate hydrochloride (1:1), Compound 3; 79.5% yield, 90.2% potency.

Notes: Alternative procedures can be employed where HCl in dioxane can be replaced by HCl in ethyl acetate or isopropyl acetate. Also dichloromethane can be replace by the same solvents (ethyl acetate or isopropyl acetate).

### Alternate Procedure Using HCl in EtOAc or IPAc as Solvent:

HCl in EtOAc or IPAc (4.5 eq.) is added dropwise to a solution of Compound 18 (1 eq.) in EtOAc (or IPAc) (1.0-2.0 L/kg of Compound 18) at 10-25 °C. The resulting mixture is stirred at 10-25 °C for 3-24 hours, and then is cooled to 0-5 °C and maintained at that temperature for at least 30 minutes. The compound tert-Butyl (2R,4R)-4-(trifluoromethyl)pyrrolidine-2-carboxylate hydrochloride (1:1) precipitates over time and is isolated by filtration and carefully washed with cold EtOAc or IPAc. (Note: addition of cool heptane during precipitation might help with impurities purge). The tert-Butyl (2R,4R)-4-(trifluoromethyl)pyrrolidine-2-carboxylate hydrochloride (1:1) is isolated as a white solid in 69-84% yield. Product Characterization: The sample contains a minor impurity making up approximately 3% of the sample. ¹H NMR (600 MHz, DMSO-d₆, 298K): δ 10.25 (br, 2H), 4.46 (t, J=8.29 Hz, 1H), 3.57 (dd, J=11.90, 9.49 Hz, 1H), 3.50 (m, 1H) 3.31 (dd, J=11.94, 7.07 Hz, 1H), 2.61 (dt, J=13.56, 8.39 Hz, 1H), 2.08 (dt, J=13.53, 8.12 Hz, 1H), 1.47 (s, 9H). ¹⁹F NMR (376 MHz, DMSO-d₆, 298K): δ −69.39 (d, J=0.03 Hz). ¹³C NMR (151 MHz, DMSO-d₆, 298K): δ 168.9 (minor), 166.5, 126.3 (1JCF=277.6 Hz), 83.6, 58.9, 58.6 (minor), 43.5, 39.9, 27.8, 27.4. HRMS: (ESI+) Calcd for C₁₀H₁₇F₃NO₂⁺: 240.1206, Found: 240.1206 (mass deviation −0.003 ppm)

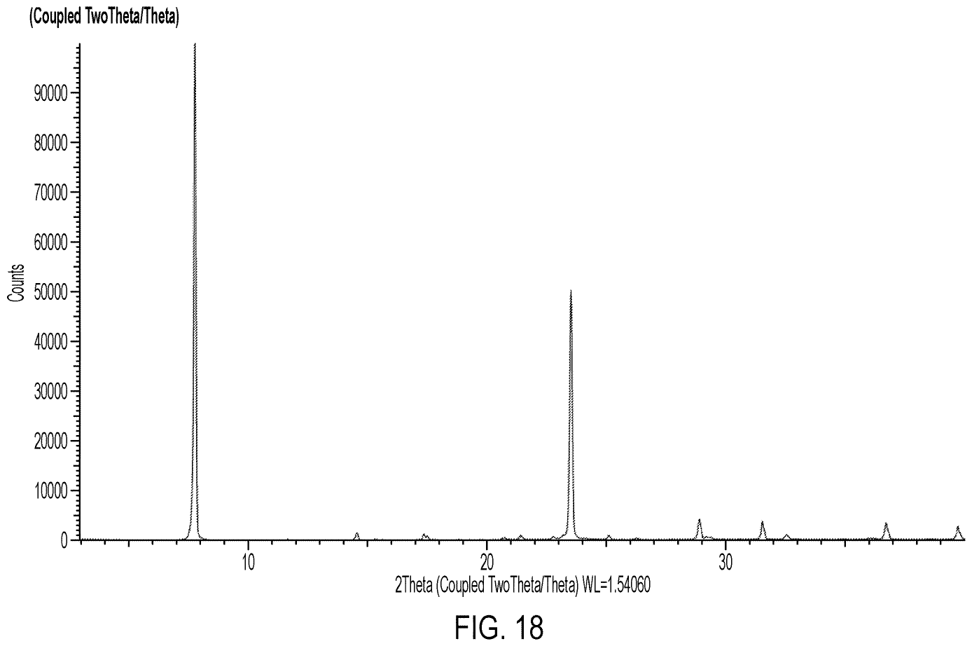

PXRD for Compound 3, tert-Butyl (2R,4R)-4-(trifluoromethyl)pyrrolidine-2-carboxylate hydrochloride (1:1) was determined (FIG. 18) and the following peaks were observed.

| Angle (°2θ ± | Rel. |
|---|---|
| 0.2 °2θ) | Intensity |
| 7.8 | 100 |
| 14.6 | 1 |
| 17.4 | 1 |
| 20.7 | 1 |
| 21.4 | 1 |
| 23.5 | 59 |
| 25.1 | 1 |
| 26.3 | 1 |
| 28.9 | 5 |
| 29.2 | 1 |
| 31.5 | 5 |
| 32.6 | 1 |
| 36.7 | 4 |
| 39.7 | 3 |

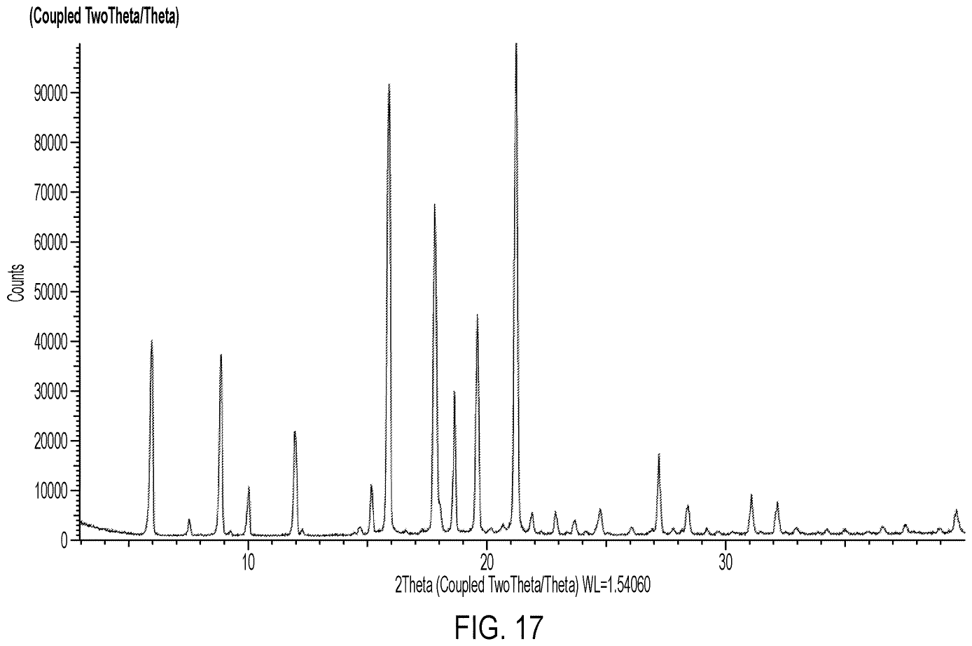

PXRD of the free form (non-salt form) of Compound 3 (FIG. 17) was also determined and the following peaks were observed.

| Angle (°2θ ± | Rel. |
|---|---|
| 0.2 °2θ) | Intensity |
| 6.0 | 37 |
| 7.6 | 3 |
| 8.9 | 35 |
| 9.2 | 1 |
| 10.1 | 9 |
| 12.0 | 21 |
| 12.3 | 1 |
| 14.5 | 1 |
| 14.7 | 1 |
| 15.2 | 10 |
| 15.9 | 87 |
| 16.6 | 1 |
| 17.3 | 1 |
| 17.8 | 65 |
| 18.7 | 30 |
| 19.6 | 45 |
| 20.2 | 1 |
| 20.7 | 2 |
| 21.2 | 100 |
| 21.9 | 4 |
| 22.9 | 5 |
| 23.7 | 3 |
| 24.7 | 5 |
| 26.1 | 1 |
| 27.2 | 18 |
| 27.8 | 1 |
| 28.4 | 5 |
| 29.2 | 1 |
| 31.1 | 9 |
| 32.2 | 6 |
| 33.0 | 1 |
| 34.3 | 1 |
| 35.0 | 1 |
| 36.6 | 1 |
| 37.5 | 2 |
| 39.0 | 1 |
| 39.7 | 5 |

### Preparation of tert-Butyl (2R,4R)-1-{(2S)-2-[(methoxycarbonyl)amino]-3,3-dimethylbutanoyl}-4-(trifluoromethyl)pyrrolidine-2-carboxylate (Compound 4)

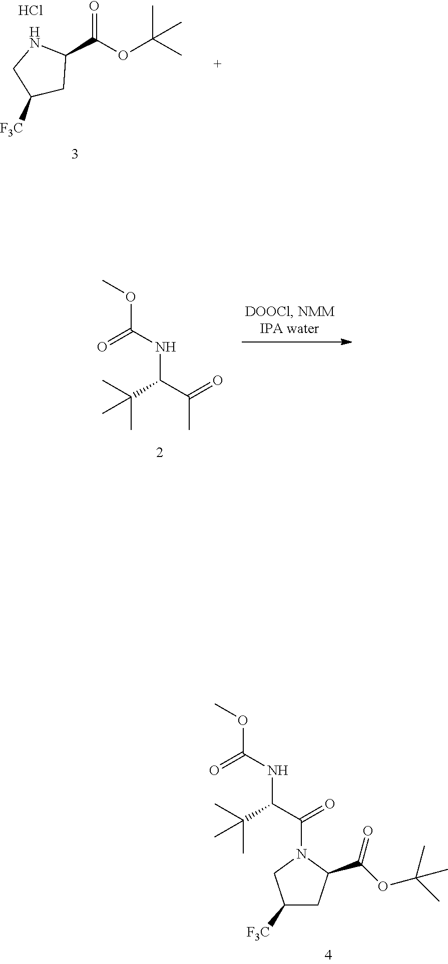 [SMILES: CC(C)(C)OC(=O)[C@H]1C[C@@H](C(F)(F)F)CN1 ; Cl ; COC(=O)N[C@H](C(C)=O)C(C)(C)C ; "DOOCl, NMMIPA water" ; COC(=O)N[C@H](C(=O)N1C[C@H](C(F)(F)F)C[C@@H]1C(=O)OC(C)(C)C)C(C)(C)C]

A flask is charged with isopropanol (30 mL, 2V), (2S)-2-[(Methoxycarbonyl)amino]-3,3-dimethylbutanoic acid, Compound 2 (11.8 g, 62.6 mmol, 1.15 eq.), tert-Butyl (2R,4R)-4-(trifluoromethyl)pyrrolidine-2-carboxylate hydrochloride (1:1), Compound 3 (15.0 g, 54.4 mmol, 1.0 eq.) and N-methylmorpholine (21.6 mL, 196 mmol, 3.6 eq.) maintaining 20 °C. To the flask is charged diphenylphosphinic chloride (13.0 mL, 68.0 mmol, 1.25 eq.) maintaining an internal temperature of 20 °C. The mixture is agitated overnight. Isopropanol (15 mL, 1 volume) is charged in a single portion at 20 °C. Water (60 mL, 4 volumes) is charged over 1 h at 20 °C. The slurry is filtered and the cake washed twice with isopropanol (12 mL, 0.8 volumes)/water (18V, 1.2 volumes). The cake is dried at >50 °C to afford tert-Butyl (2R,4R)-1-{(2S)-2-[(methoxycarbonyl)amino]-3,3-dimethylbutanoyl}-4-(trifluoromethyl)pyrrolidine-2-carboxylate, Compound 4, as a white crystalline solid (19.6 g, 87% yield). ¹H NMR (600 MHz, DMSO-d6) δ 7.58 (d, J=9.77 Hz, minor), 7.30 (d, J=9.68 Hz, 1H), 6.84 (br d, J=9.13 Hz, minor), 4.88 (dd, J=9.96, 3.14 Hz, minor), 4.29 (d, J=9.65 Hz, 1H), 4.27 (dd, J=8.86, 6.76 Hz, 1H), 4.24 (dd, J=10.47, 8.30 Hz, 1H), 4.16 (br d, J=8.59 Hz, minor), 4.07 (dd, J=12.72, 9.27 Hz, minor), 3.93 (d, J=9.67 Hz, minor), 3.51-3.60 (om, 1H), 3.55 (s, minor), 3.54 (s, 3H), 3.44 (m, 1H), 3.31 (m, minor), 3.19 (dd, J=12.55, 7.05 Hz, minor), 2.70 (dt, J=14.27, 10.03 Hz, minor), 2.59 (dt, J=13.17, 8.96 Hz, 1H), 2.12 (ddd, J=14.26, 4.62, 3.36 Hz, minor), 1.79 (ddd, J=13.35, 8.70, 6.84 Hz, 1H), 1.43 (s, minor), 1.34 (s, 9H), 0.94 (s, 9H), 0.93 (s, minor). ¹³C NMR: (151 MHz, DMSO-d6) δ 170.3 (minor), 169.7, 169.6 (minor), 169.6, 157.1 (minor), 156.8, 126.4 (¹J_{CF}=277.1 Hz), 127.0 (¹J_{CF}=278.2 Hz, minor), 81.8 (minor), 80.4, 58.9 (minor), 58.8, 58.3, 57.8 (minor), 51.7 (minor), 51.4, 46.3, 46.1 (minor), 44.5, 40.6 (²J_{CF}=28.2 Hz), 38.2 (²J_{CF}=27.8 Hz), 34.2 (minor), 34.1, 34.0 (minor), 29.7 (minor), 27.6, 27.3 (minor), 27.2, 26.3 (minor), 26.0. ¹⁹F NMR: (376 MHz, DMSO-d6) δ −70.06, −70.11 (minor), −70.18 (minor). HRMS (ESI) calculated for C₁₈H₂₉F₃N₂O₅ [M+H]⁺ 411.2101 found 411.2105.

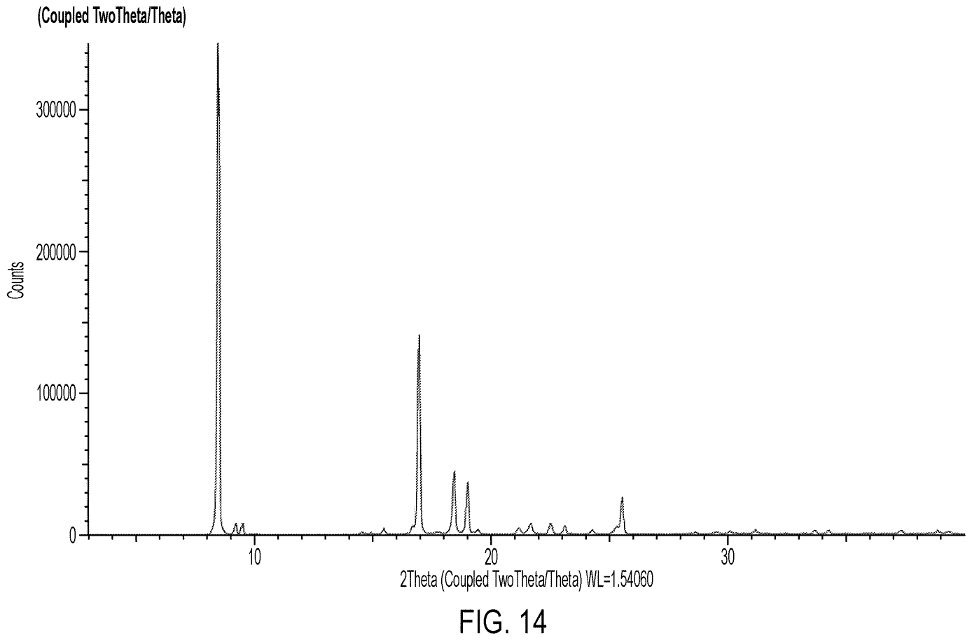

PXRD for Compound 4 was determined (FIG. 14) and the peaks in the following table were observed.

| Angle (°2θ ± | Rel. |
|---|---|
| 0.2 °2θ) | Intensity |
| 7.7 | 0 |
| 8.4 | 100 |
| 9.2 | 2 |
| 9.5 | 2 |
| 9.9 | 1 |
| 10.1 | 1 |
| 14.6 | 1 |
| 14.9 | 1 |
| 15.5 | 1 |
| 16.7 | 2 |
| 17.0 | 37 |
| 17.8 | 1 |
| 18.5 | 12 |
| 19.0 | 11 |
| 19.4 | 1 |
| 21.2 | 1 |
| 21.7 | 2 |
| 22.5 | 2 |
| 23.1 | 2 |
| 24.3 | 1 |
| 25.3 | 1 |
| 25.6 | 7 |
| 28.7 | 1 |
| 29.6 | 1 |
| 30.1 | 1 |
| 31.2 | 1 |
| 33.7 | 1 |
| 34.3 | 1 |
| 37.4 | 1 |
| 38.9 | 1 |
| 39.3 | 1 |

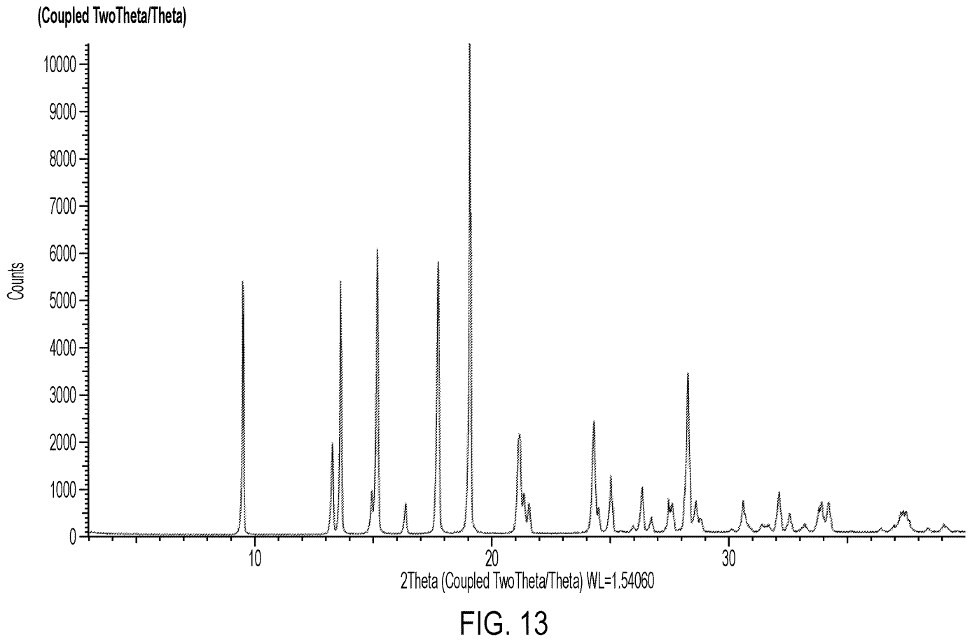

PXRD of Compound 2 was determined (FIG. 13) and the peaks in the following table were observed.

| Angle (°2θ ± | Rel. |
|---|---|
| 0.2 °2θ) | Intensity |
| 9.5 | 36 |
| 13.3 | 12 |
| 13.6 | 39 |
| 14.9 | 6 |
| 15.2 | 39 |
| 16.4 | 4 |
| 17.7 | 39 |
| 19.1 | 100 |
| 21.2 | 12 |
| 21.4 | 6 |
| 21.6 | 4 |
| 24.3 | 16 |
| 24.5 | 4 |
| 25.0 | 8 |
| 26.3 | 7 |
| 27.6 | 4 |
| 28.3 | 24 |
| 28.6 | 5 |
| 30.6 | 5 |
| 32.1 | 6 |
| 32.6 | 3 |
| 33.8 | 4 |
| 33.9 | 4 |
| 34.2 | 4 |
| 37.3 | 3 |
| 37.4 | 3 |

### EXAMPLES

### Example 1

### Preparation of Potassium (2S,4R)-1-{(2S)-2-[(methoxycarbonyl)amino]-3,3-dimethylbutanoyl}-4-(trifluoromethyl)pyrrolidine-2-carboxylate (Compound 6)

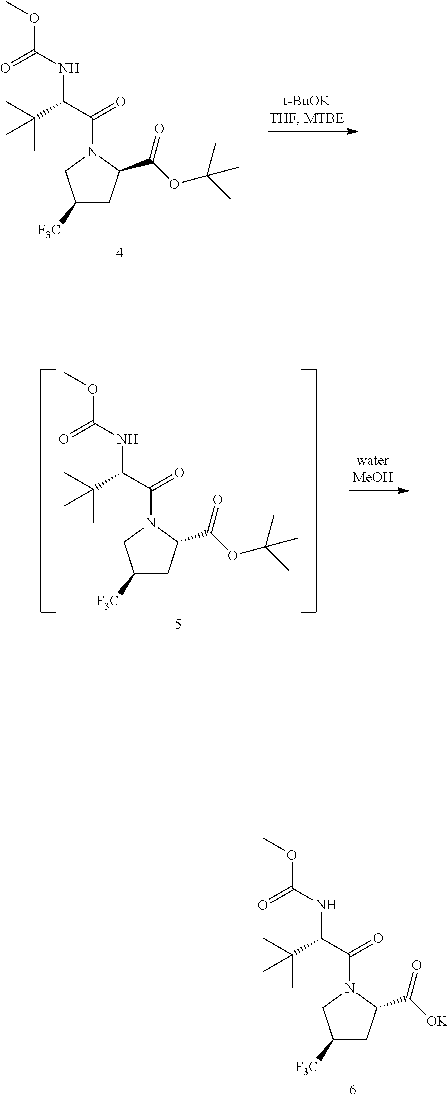

A flask is charged with MTBE (10 mL, 2.5 volumes), tert-Butyl (2R,4R)-1-{(2S)-2-[(methoxycarbonyl)amino]-3,3-dimethylbutanoyl}-4-(trifluoromethyl)pyrrolidine-2-carboxylate, Compound 4 (4.0 g, 9.6 mmol, 1.0 eq.) and water (0.21 mL, 12 mmol, 1.2 eq.) at 20 °C. A solution of potassium tert-butoxide, KOt-Bu (20 wt % in THF, 7.0 mL, 12 mmol, 1.2 eq.) is charged to the flask maintaining an internal temperature of 20 °C. The mixture is agitated overnight. Methanol (2.0 mL, 0.5 volumes) is charged in a single portion at 20 °C. The mixture is agitated. The slurry is filtered and the cake washed with MTBE (6.7 mL, 1.7 volumes)/methanol (1.3 mL, 0.3 volumes). The cake is dried at >60 °C to afford Potassium (2S,4R)-1-{(2S)-2-[(methoxycarbonyl)amino]-3,3-dimethylbutanoyl}-4-(trifluoromethyl)pyrrolidine-2-carboxylate, Compound 6 as a white crystalline solid (3.39 g, 87% yield). ¹H NMR (600 MHz, CD₃OD) δ 4.56 (dd, J=6.81, 4.59 Hz, 1H, E), 4.51 (dd, J=8.71, 3.97 Hz, 1H, Z), 4.30 (s, 1H, Z), 4.27 (s, 1H, E), 4.24 (br, minor), 4.03 (dd, J=10.77, 8.23 Hz, 1H, Z), 3.96 (dd, J=10.67, 6.72 Hz, 1H, Z), 3.87 (dd, J=12.57, 8.67 Hz, 1H, E), 3.68 (br s, minor), 3.65 (s, 3H, Z), 3.63 (s, 3H, E), 3.59 (dd, J=12.62, 6.91 Hz, 1H, E), 3.29 (m, 1H, Z), 3.15 (m, 1H, E), 2.34-2.40 (om, 2H, E), 2.28 (m, 1H, Z), 2.23 (m, 1H, Z), 1.06 (s, 9H, Z), 0.97 (s, 9H, E). ¹³C NMR (151 MHz, CD₃OD) δ 178.5 (Z), 177.9 (E), 177.4 (minor), 172.4 (E), 172.3 (minor), 171.9 (Z), 159.5 (Z), 158.7 (E), 158.4 (minor), 128.6 (¹J_{CF}=276.3 Hz, E), 128.5 (¹J_{CF}=276.6 Hz, Z), 63.9 (E), 62.7 (Z), 61.1 (minor), 60.8 (minor), 60.7 (Z), 60.5 (E), 53.1 (minor), 52.9 (Z), 52.8 (E), 48.2 (Z), 46.8 (E), 43.3 (²J_{CF}=28.8 Hz, Z), 41.1 (²J_{CF}=29.0 Hz, E), 37.2 (E), 36.8 (minor), 36.5 (Z), 32.4 (minor), 32.3 (E), 30.3 (Z), 27.1 (Z), 26.9 (E), 26.8 (minor). ¹⁹F NMR (376 MHz, CD₃OD) δ −72.90, −73.15. HRMS (ESI) calculated for C₁₄H₂₁F₃N₂O₅ [M+H]+ 355.1475 found 355.1471.

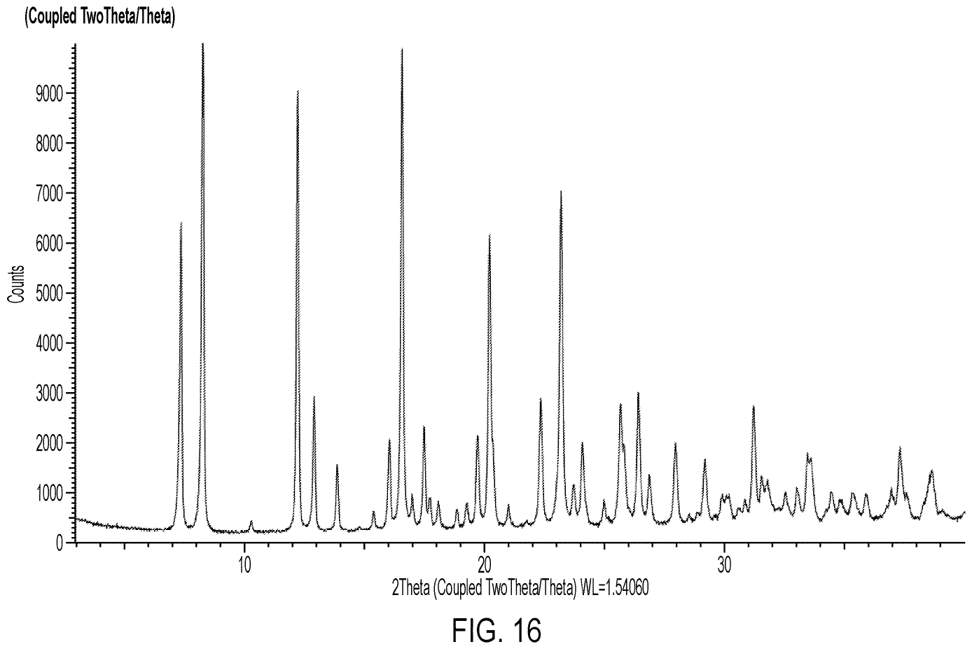

PXRD was determined (FIG. 16) and the peaks in the following table were observed.

| Angle (°2θ ± | Rel. |
|---|---|
| 0.2 °2θ) | Intensity |
| 7.4 | 60 |
| 8.3 | 100 |
| 10.3 | 2 |
| 12.2 | 89 |
| 12.9 | 27 |
| 13.9 | 14 |
| 14.8 | 1 |
| 15.4 | 4 |
| 16.1 | 18 |
| 16.6 | 99 |
| 17.0 | 7 |
| 17.5 | 21 |
| 17.7 | 6 |
| 18.1 | 6 |
| 18.9 | 4 |
| 19.3 | 5 |
| 19.7 | 18 |
| 20.2 | 63 |
| 20.3 | 15 |
| 21.0 | 5 |
| 22.4 | 27 |
| 23.2 | 73 |
| 23.7 | 8 |
| 24.1 | 18 |
| 25.0 | 5 |
| 25.7 | 26 |
| 25.8 | 17 |
| 26.4 | 30 |
| 26.9 | 11 |
| 28.0 | 18 |
| 29.2 | 13 |
| 29.9 | 5 |
| 30.1 | 5 |
| 30.2 | 5 |
| 30.9 | 4 |
| 31.2 | 27 |
| 31.5 | 10 |
| 31.8 | 7 |
| 32.6 | 5 |
| 33.0 | 7 |
| 33.5 | 15 |
| 33.6 | 12 |
| 34.5 | 6 |
| 34.8 | 4 |
| 34.9 | 3 |
| 35.4 | 5 |
| 35.9 | 5 |
| 36.8 | 3 |
| 37.0 | 7 |
| 37.3 | 16 |
| 37.6 | 5 |
| 38.3 | 4 |
| 38.6 | 8 |
| 38.6 | 9 |

### Example 2

### Preparation of Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate ethyl acetate solvate, (Compound I ethyl acetate solvate)

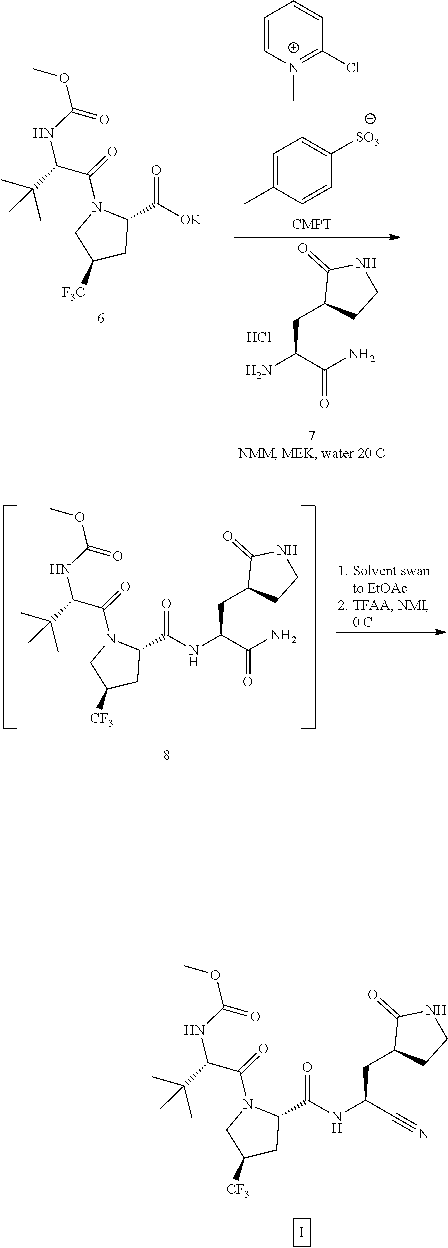

The Compound I shown in the Scheme above is isolated as the EtOAc solvate form. To a 100 mL vessel with overhead stirring was charged 2-butanone (40 ml-) and Potassium (2S,4R)-1-{(2S)-2-[(methoxycarbonyl)amino]-3,3-dimethylbutanoyl}-4-(trifluoromethyl)pyrrolidine-2-carboxylate, Compound 6 (4.04 g, 9.98 mmol) and the slurry was stirred at 20 °C. N-methylmorpholine (3.3 mL, 30 mmol, 3 eq.), 2-chloro-1-methylpyridinium p-toluenesulfonate (2.63 g, 11.9 mmol, 1.2 eq.), (2S)-2-Amino-3-[(3S)-2-oxopyrrolidin-3-yl]propanamide hydrochloride (1:1), Compound 7 (2.36 g, 11.37 mmol, 1.15 eq.) and water (1.2 mL) were charged. The reaction was stirred at 20 °C for 4 h to provide Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate, Compound 8 which was carried forward without isolation.

A sample of Compound 8 was isolated for characterization. Compound 8 Characterization: ¹H NMR (600 MHz, DMSO-d₆) δ ppm 0.95 (s, 10H), 1.18 (t, J=7.34 Hz, 2H), 1.45-1.52 (m, 1H), 1.57-1.67 (m, 1H), 1.89-1.98 (m, 1H), 2.08-2.16 (m, 2H), 2.21-2.28 (m, 1H), 2.39-2.46 (m, 1H), 3.00-3.17 (m, 3H), 3.34-3.40 (m, 1H), 3.52 (s, 3H), 4.15 (br d, J=8.80 Hz, 1H), 4.25 (ddd, J=12.10, 8.80, 3.48 Hz, 1H), 4.51 (dd, J=8.07, 5.69 Hz, 1H), 7.04 (s, 1H), 7.25 (br d, J=8.62 Hz, 1H), 7.33 (s, 1H), 7.54 (s, 1H), 8.28 (d, J=8.80 Hz, 1H). ¹³C NMR (151 MHz, DMSO-d6) b ppm 9.03 (s, 1 C), 26.64 (s, 3 C), 27.88 (s, 1 C), 28.79 (s, 1 C), 34.57 (s, 1 C), 34.94 (s, 2 C), 37.70 (s, 1 C), 41.76 (s, 1 C), 46.03 (s, 1 C), 50.83 (s, 1 C), 51.97 (s, 1 C), 59.20 (s, 1 C), 59.40 (s, 1 C), 128.49 (s, 1 C), 157.46 (s, 1 C), 170.13 (s, 1 C), 171.36 (s, 1 C), 173.89 (s, 1 C), 179.07 (s, 1 C).

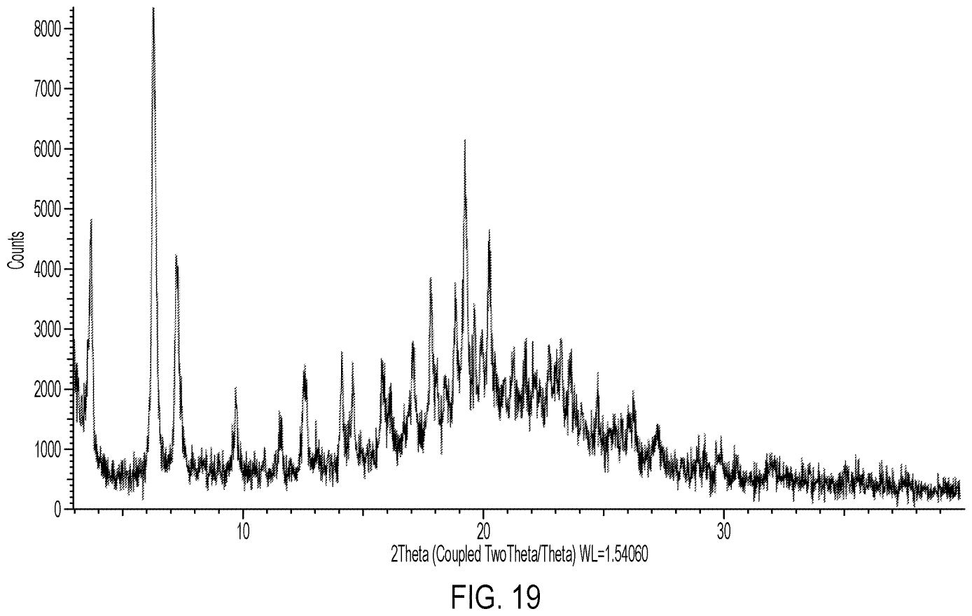

PXRD was determined for Compound 8 (FIG. 19) and the peaks in the following table were observed.

| Angle (°2θ ± | Rel. |
|---|---|
| 0.2 °2θ) | Intensity |
| 3.7 | 37 |
| 6.3 | 100 |
| 7.3 | 35 |
| 9.7 | 18 |
| 11.6 | 11 |
| 12.6 | 19 |
| 14.1 | 25 |
| 14.6 | 20 |
| 15.8 | 13 |
| 16.2 | 12 |
| 17.1 | 11 |
| 17.9 | 28 |
| 18.5 | 4 |
| 18.9 | 22 |
| 19.2 | 59 |
| 19.6 | 32 |
| 20.0 | 18 |
| 20.3 | 35 |
| 20.9 | 8 |
| 21.2 | 8 |
| 21.7 | 18 |
| 22.8 | 11 |
| 23.2 | 21 |
| 23.6 | 15 |
| 24.7 | 14 |
| 26.1 | 9 |
| 26.2 | 16 |
| 29.8 | 7 |

The crude reaction mixture from the first step above was solvent exchanged into ethyl acetate (40 mL) by vacuum distillation. Distillation was continued until the water content was <0.2 wt %. The slurry was cooled to 0 °C and N-methylimidazole (4 mL, 50.2 mmol, 5 eq.) was charged. Trifluoroacetic anhydride (3.5 mL, 25 mmol, 2.5 eq.) was added over 30 min and the reaction stirred at 0 °C for 1 h. The reaction was quenched with a mixture of citric acid monohydrate (8.42 g, 39.67 mmol, 4 eq.) and sodium chloride (6.22 g, 106.5 mmol, 10.7 eq.) in water (40 mL). The phases were separated and the aqueous layer was back-extracted with ethyl acetate (40 mL). The combined organic layers were washed with a mixture of potassium phosphate dibasic (13.93 g, 80 mmol, 8 eq.) in water (40 mL) followed by 14% sodium chloride in water (20 mL). The organic layer was distilled under vacuum to 20 mL and held at 45 °C. The product Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate ethyl acetate solvate, Compound I ethyl acetate solvate was isolated as solids from a mixture of ethyl acetate and heptane (3.70 g, 7.32 mmol, 73% yield):

Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate ethyl acetate solvate:

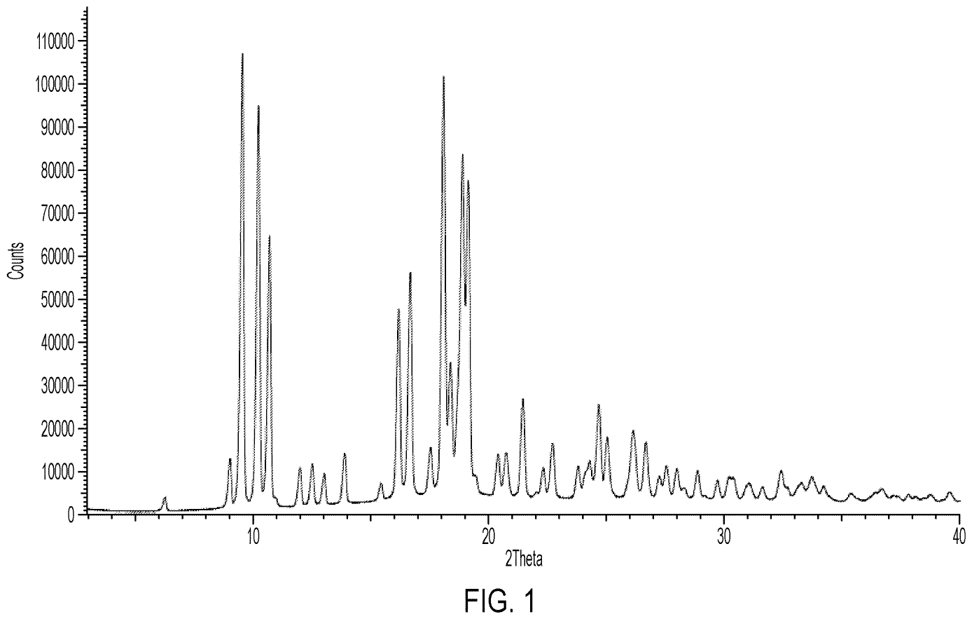

The sample contains a major rotamer (~83.2%, shown in FIG. 1) and a minor rotamer (~7.0%) due to E/Z isomerization of the N14-C18 amide bond, and a third minor rotamer (~9.8%) due to the N24-C25 carbamate amide bond.

¹H NMR (600 MHz, DMSO-d6) δ 9.09 (d, J=6.90 Hz, minor), 9.02 (d, J=8.52 Hz, 1H), 7.72 (s, minor), 7.66 (s, 1H), 7.27 (d, J=8.83 Hz, 1H), 6.95 (d, J=8.96 Hz, minor), 6.85 (br, minor), 4.95 (ddd, J=10.95, 8.54, 4.95 Hz, 1H), 4.89 (q, J=7.81 Hz, minor), 4.80 (dd, J=8.52, 3.06 Hz, minor), 4.36 (dd, J=8.23, 6.29 Hz, 1H), 4.15 (d, J=8.77 Hz, 1H), 4.07 (br, minor), 4.04 (d, J=8.74 Hz, minor), 3.96 (m, 2H), 3.85 (br, minor), 3.66 (m, minor), 3.52 (s, 3H), 3.50 (s, minor), 3.41 (m, 1H), 3.30-3.35 (m, overlapped with residual water, minor), 3.12-3.20 (om, minor), 3.14 (m, 1H), 3.04 (td, J=9.39, 7.08 Hz, 1H), 2.45 (m, 1H), 2.30 (m, 1H), 2.16 (ddd, J=13.64, 11.03, 4.43 Hz, 1H), 2.05-2.12 (om, 2H), 1.84 (dt, J=13.61, 7.91 Hz, minor), 1.65-1.73 (om, 2H), 0.94 (s, 9H), 0.89 (s, minor). ¹³C NMR (151 MHz, DMSO-d6) δ 177.5 (minor), 177.4, 170.8, 170.4 (minor), 169.8, 169.4 (minor), 156.9, 156.3 (minor), 127.1 (¹J_{CF}=278.4 Hz, minor), 126.9 (¹J_{CF}=277.3 Hz), 119.5, 119.2 (minor), 58.9, 58.5, 58.4 (minor), 51.4, 51.3 (minor), 46.7, 45.3 (minor), 41.2 (²J_{CF}=27.8 Hz), 39.1 (overlapped with solvent), 37.6, 37.3 (minor), 36.5, 35.1 (minor), 34.3, 34.1, 33.2 (minor), 30.4 (minor), 28.1, 27.3 (minor), 26.8, 26.0, 25.9 (minor). ¹⁹F NMR (376 MHz, DMSO-d₆) δ −70.16, −70.44 (minor), −70.57 (minor). om=overlapped multiplet, br=broad signal. HRMS (ESI) m/z: [M+H]⁺ calculated for C₂₁H₃₁O₅N₅F₃ 490.2272 Da, found 490.2272 Da.

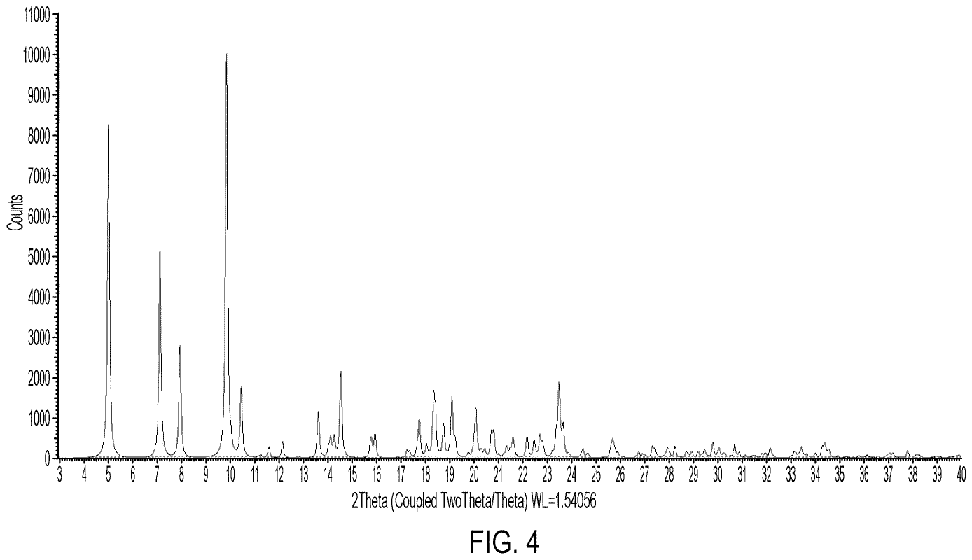

PXRD Pattern for Compound I EtOAc solvate is shown in FIG. 4.

### Example 3

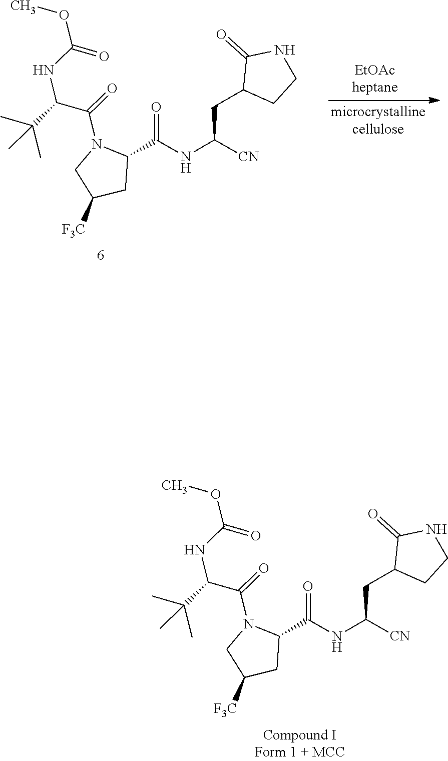 [SMILES: COC(=O)N[C@H](C(=O)N1C[C@H](C(F)(F)F)C[C@H]1C(=O)N[C@H](C#N)CC1CCNC1=O)C(C)(C)C ; COC(=O)N[C@H](C(=O)N1C[C@H](C(F)(F)F)C[C@H]1C(=O)N[C@H](C#N)CC1CCNC1=O)C(C)(C)C]

### Procedure:

To an appropriately sized vessel at 25 °C was charged 135 kg of EtOAc, 34.2 kg of heptane, and 25.0 kg Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate, Form 1, with stirring at 120 rpm. The mixture is then heated to 60 °C and agitated for 10 min until a homogeneous solution is achieved. The mixture is then cooled to 45 °C, and 2.78 kg of microcrystalline cellulose (MCC) is subsequently charged. The mixture is then stirred at 45 °C for 30 minutes. Then 120 kg additional heptane is charged over a period of 4 h, and subsequently mixed at 45 °C for 30 min. The mixture is cooled to 20 °C and held at this temperature for 12 h. A constant volume vacuum distillation is then performed using 342 kg added heptane to remove EtOAc, maintaining a temperature difference between reactor and mixture of 25 °C, and reaching a final internal mixture temperature of 52 °C. The mixture is then cooled to 25 °C once again and sampled for EtOAc content. The batch is then heated once again to 80 °C and held at this temperature for 6 h. The mixture is sampled to confirm PXRD consistency with desired Form 1+MCC, and subsequently cooled to 20 °C. The batch is granulated at 20 °C for 1 h, and then filtered. The vessel is rinsed with 68 kg heptane, and this rinse is then applied to the filter cake to wash it. The filter cake is then dried and isolated to provide Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1+MCC.

### Product Characterization:

Note: The sample contains two minor rotamers due to the N1-C10 and N16-C17 amide bonds, making up approximately 6% and 10% of the sample, respectively. Where relevant, the less abundant rotamer signals are designated as “minor.”

¹H NMR (600 MHz, DMSO-d₆, 298K): δ 9.11 (d, J=6.97 Hz, minor), 9.05 (d, J=8.63 Hz, 1H), 7.75 (s, minor), 7.69 (s, 1H), 7.32 (d, J=8.66 Hz, 1H), 7.01 (d, J=9.16 Hz, minor), 6.90 (br, minor), 4.95 (ddd, J=11.23, 8.63, 4.86 Hz, 1H), 4.88 (q, J=7.53 Hz, minor), 4.80 (dd, J=8.16, 2.90 Hz, minor), 4.35 (t, J=7.53 Hz, 1H), 4.14 (d, J=8.78 Hz, 1H), 4.07 (br, minor), 4.04 (d, J=9.10 Hz, minor), 3.98-3.95 (m, 1H), 3.84 (br, minor), 3.65 (d, J=7.06 Hz, minor), 3.52 (s, 3H), 3.49 (s, minor), 3.41 (m, 1H), 3.31 (m, minor), 3.17 (m, minor), 3.13 (t, J=9.11 Hz, 1H), 3.03 (td, J=9.32, 7.17 Hz, 1H), 2.46 (m, 1H), 2.30 (m, 1H), 2.24 (m, minor), 2.16 (ddd, J=13.51, 11.25, 4.28 Hz, 1H), 2.12-2.05 (om, 2H), 1.84 (m, minor), 1.73-1.65 (om, 2H), 0.94 (s, 9H), 0.89 (s, minor). ¹³C NMR (151 MHz, DMSO-d₆, 298K): d 177.5 (minor), 177.4, 170.8, 170.4 (minor), 169.9 (minor), 169.7, 169.3 (minor), 156.9, 156.3 (minor), 155.5 (minor), 127.0 (1JCF=278.4 Hz), 119.5, 119.2 (minor), 59.4 (minor), 58.9, 58.8 (minor), 58.6 (minor), 58.5, 58.3 (minor), 51.6 (minor), 51.4, 51.3 (minor), 46.8 (minor), 46.7, 45.3 (minor), 41.1 (2JCF=28.0 Hz), 39.5 (minor, ov), 39.1, 39.0 (minor, ov), 38.6 (2JCF=27.8 Hz, minor), 37.6, 37.3 (minor), 36.5, 35.1 (minor), 34.3, 34.2 (minor), 34.1, 33.2 (minor), 30.3 (minor), 28.1, 27.3 (minor), 26.7, 26.0, 25.9 (minor). ¹⁹F NMR (376 MHz, DMSO-d₆, 298K): d −70.17, −70.45 (minor), −70.58 (minor). om=overlapped multiplet, ov=overlapped with solvent, br=broad signal. HRMS: (ESI) m/z: [M+H]⁺ calculated for C₂₁H₃₁O₅N₅F₃ 490.2272 Da, found 490.2271 Da. Additional lots designated A, B and C of Methyl {(2S)-1-[(2S,4R)-2-({(1 S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1+microcrystalline cellulose (90/10 weight %) were prepared in an analogous fashion on an 80 gram scale and the resulting particle size distribution was analyzed. The D[10], D[50] and D[90] values in μm for these lots is provided below.

| Lot | D[10] μm | D[50] μm | D[90] μm |
|---|---|---|---|
| A | 11.80 | 65.59 | 165.98 |
| B | 14.33 | 75.78 | 188.35 |
| C | 10.06 | 51.16 | 164.39 |

Further additional lots, D-Trays 1-3 and E, Methyl {(2S)-1-[(2S,4R)-2-({(I S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate Form 1+microcrystalline cellulose (90/10 weight %) were prepared in an analogous fashion and the resulting particles were analyzed.

| | | | | | Aspect | | Surface | Bulk | Tapped |
|---|---|---|---|---|---|---|---|---|---|
| | D[10] | D[50] | D[90] | D[4, 3] | Ratio | Agglom- | Area | Density | Density |
| Lot | μm | μm | μm | μm | AR50 | erates | (m²/g) | (g/cm³) | (g/cm³) |
| D- | 10.63 | 68.49 | 175.98 | 84.41 | 0.68 | Soft up | 2.52 | 0.356 | 0.498 |
| Tray | | | | | | to | | | |
| 1 | | | | | | 2 mm | | | |
| D- | 10.48 | 69.01 | 178.94 | 85.58 | 0.68 | Soft up | 2.31 | | |
| Tray | | | | | | to | | | |
| 2 | | | | | | 1 mm | | | |
| D- | 10.25 | 70.05 | 182.99 | 87.23 | 0.68 | Soft | 2.46 | | |
| Tray | | | | | | up to | | | |
| 3 | | | | | | 12.5 | | | |
| | | | | | | mm | | | |
| E | 22.22 | 72.69 | 165.58 | 86.98 | 0.68 | None | 0.80 | 0.398 | 0.548 |

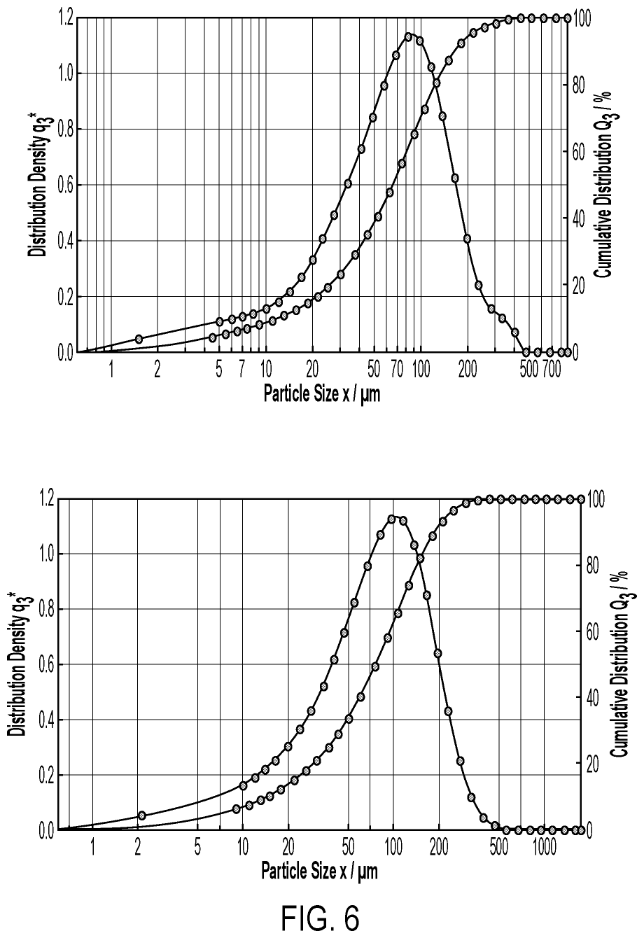

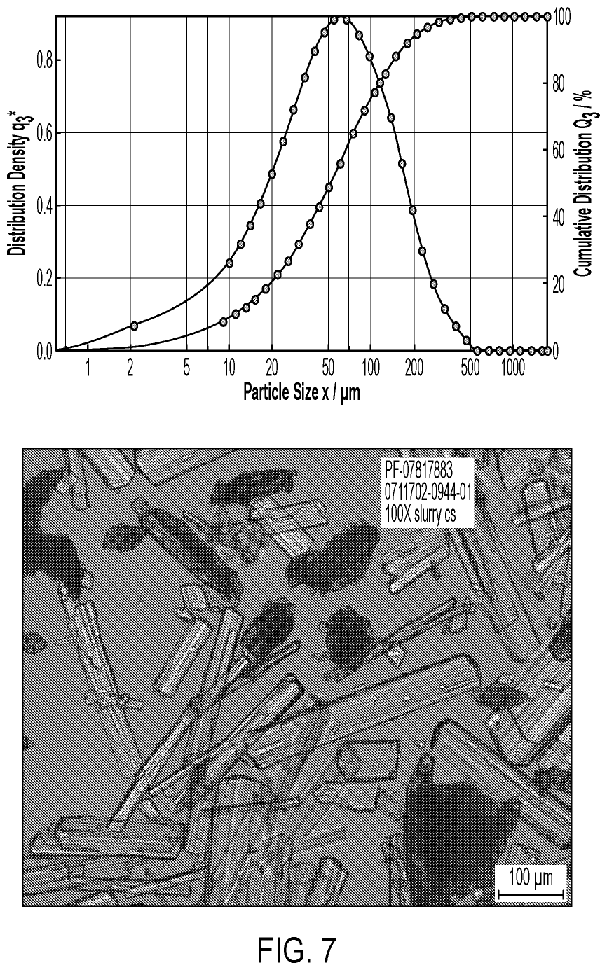

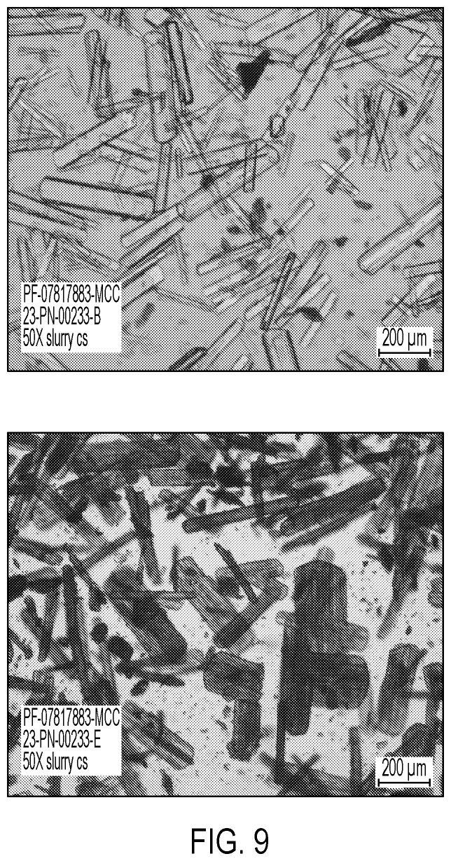

The lots D-Tray 1-3 and E were also found to have a flow function coefficient of 18.6 and 69.0, respectively. Particle size and cumulative distribution for certain lots as well as images of the Compound I Form 1+MCC are provided in FIGS. 6-9.

The above data indicate that Compound I Form 1 with MCC additive can be prepared in the desired form and with a desired particle size distribution useful as a drug product intermediate.

Additional characterizing data for Compound I co-processed with MCC is described below.

Powder X-Ray Diffraction (PXRD) and ssNMR Data of Co-Processed Form 1 with 10 wt % MCC

### Instrumentation

### Powder X-Ray Diffraction (PXRD):

Powder X-ray diffraction analysis was conducted using a Bruker AXS D8 Endeavor diffractometer equipped with a Cu radiation source (K-α average). The divergence slit was set at 15 mm continuous illumination. Diffracted radiation was detected by a PSD-Lynx Eye detector, with the detector PSD opening set at 4.10 degrees. The X-ray tube voltage and amperage were set to 40 kV and 40 mA respectively. In addition, the energy dispersive detector, a nickel filter was used to screen out unwanted wavelengths. Data was collected in the Theta-Theta goniometer at the Cu wavelength from 3.0 to 40.0 degrees 2-Theta using a step size of 0.01 degrees and a step time of 1.0 second. The anti-scatter screen was set to a fixed distance of 3.0 mm. Samples were rotated at 15/min during collection. Samples were prepared by placing them in a silicon low background sample holder and rotated during collection. Data were collected using Bruker DIFFRAC Plus software and analysis was performed by EVA diffract plus software.

The PXRD data file was not processed prior to peak searching. Using the peak search algorithm in the EVA software, peaks selected with a threshold value of 1 were used to make preliminary peak assignments. To ensure validity, adjustments were manually made; the output of automated assignments was visually checked, and peak positions were adjusted to the peak maximum. Peaks with relative intensity of >3% were generally chosen. The peaks which were not resolved or were consistent with noise were not selected. A typical error associated with the peak position from PXRD stated in USP up to +/−0.2° 2-Theta (USP-941).

Solid-State Nuclear Magnetic Resonance (ssNMR):

¹³C solid-state NMR (ssNMR) analysis was conducted on a CPMAS probe positioned into a Bruker-BioSpin Avance NEO 500 MHz (¹H frequency) NMR spectrometer. ¹⁹F solid-state NMR (ssNMR) analysis was conducted on a CPMAS probe positioned into a Bruker-BioSpin Avance III 600 MHz (¹H frequency) NMR spectrometer. Material was packed into a ZrO₂ rotor. A magic angle spinning rate of 15 kHz was used. Spectra were collected at ambient temperature (probe temperature of 25 °C.)

¹³C ssNMR spectra were collected using a proton decoupled cross-polarization magic angle spinning (CPMAS) experiment. A phase modulated proton decoupling field of 80-100 kHz was applied during spectral acquisition. The cross-polarization contact time was set to 2 ms and the recycle delay to 3.5 seconds. The number of scans was adjusted to obtain an adequate signal to noise ratio. The ¹³C chemical shift scale was referenced using a ¹³C CPMAS experiment on an external standard of crystalline adamantane, setting its up-field resonance to 29.5 ppm.

¹⁹F ssNMR spectra were collected using a proton decoupled magic angle spinning (MAS) experiment. A phase modulated proton decoupling field of 80-100 kHz was applied during spectral acquisition. Spectra were collected with a recycle delay of 3.5 seconds. The number of scans was adjusted to obtain an adequate signal to noise ratio. The ¹⁹F chemical shift scale was referenced using a ¹⁹F MAS experiment on an external standard of trifluoroacetic acid (50%/50% v/v in H₂O), setting its resonance to −76.54 ppm.

Automatic peak picking was performed using Bruker-BioSpin TopSpin version 4.1 software. Generally, a threshold value of 5% relative intensity was used for preliminary peak selection. The output of the automated peak picking was visually checked to ensure validity and adjustments were manually made if necessary. Although specific solid-state NMR peak values are reported herein there does exist a range for these peak values due to differences in instruments, samples, and sample preparation. This is common practice in the art of solid-state NMR because of the variation inherent in peak positions. A typical variability for the chemical shift x-axis value is on the order of plus or minus 0.2 ppm for a crystalline solid. The solid-state NMR peak heights reported herein are relative intensities. Solid-state NMR intensities can vary depending on the actual setup of the experimental parameters and the thermal history of the sample.

### Results

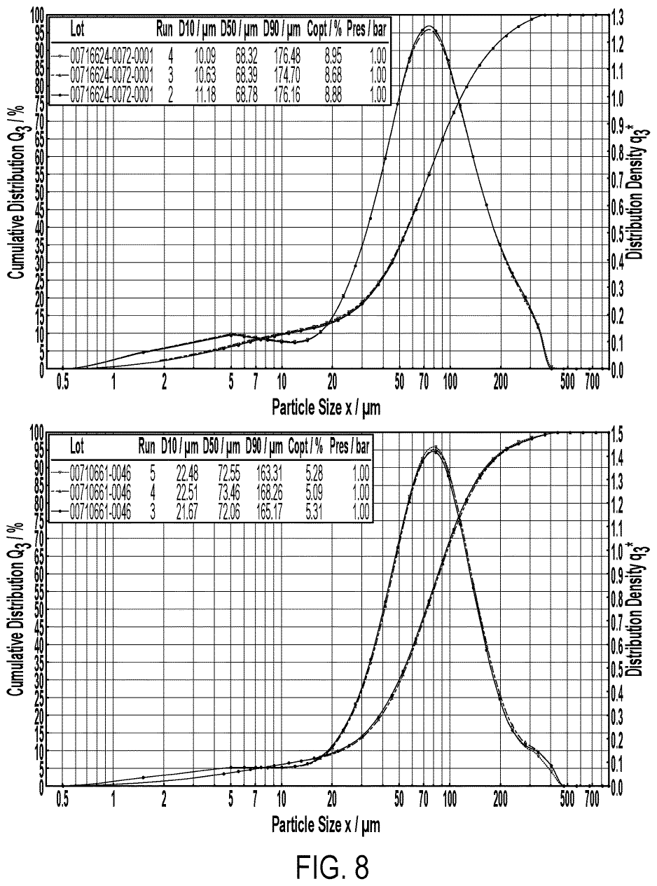

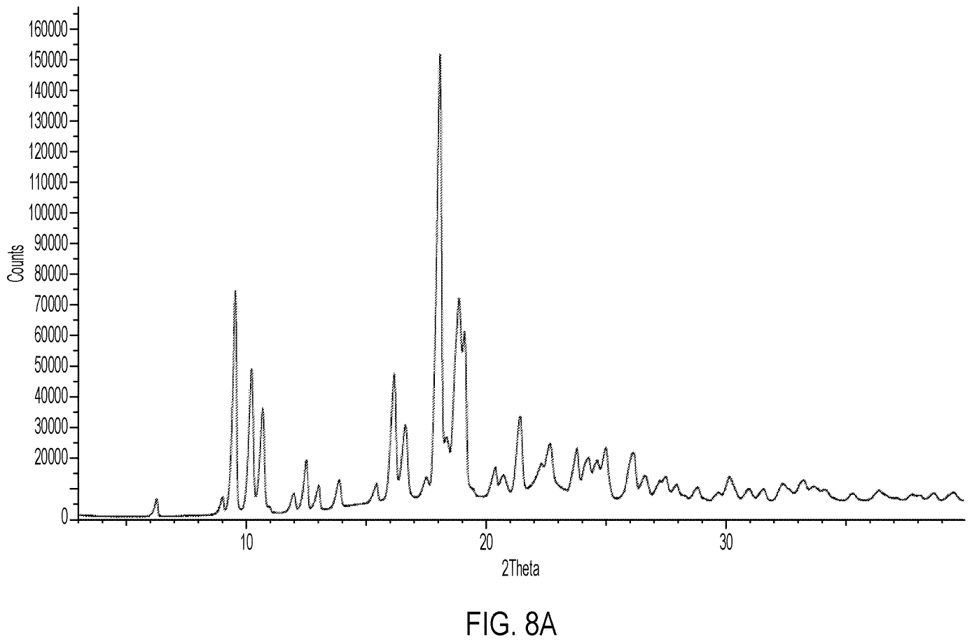

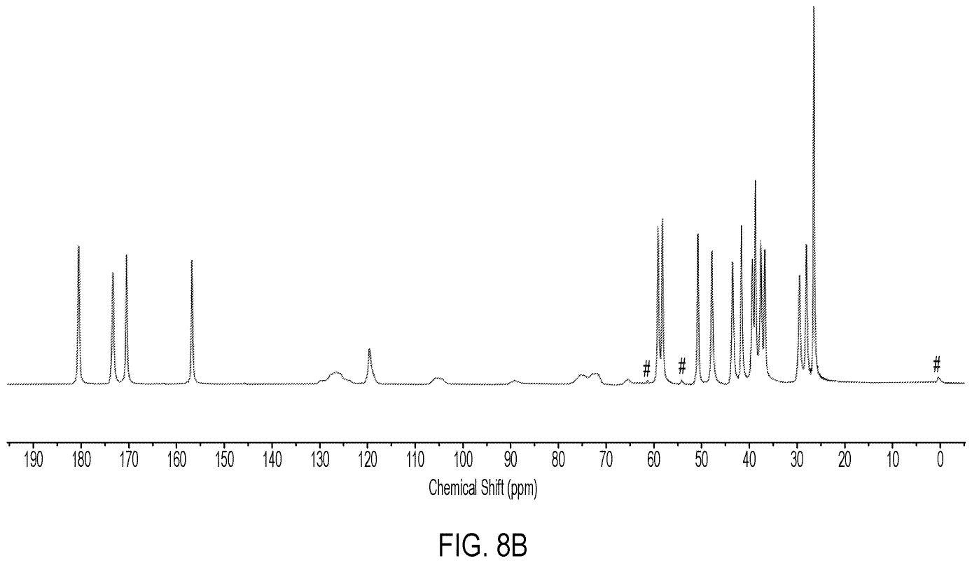

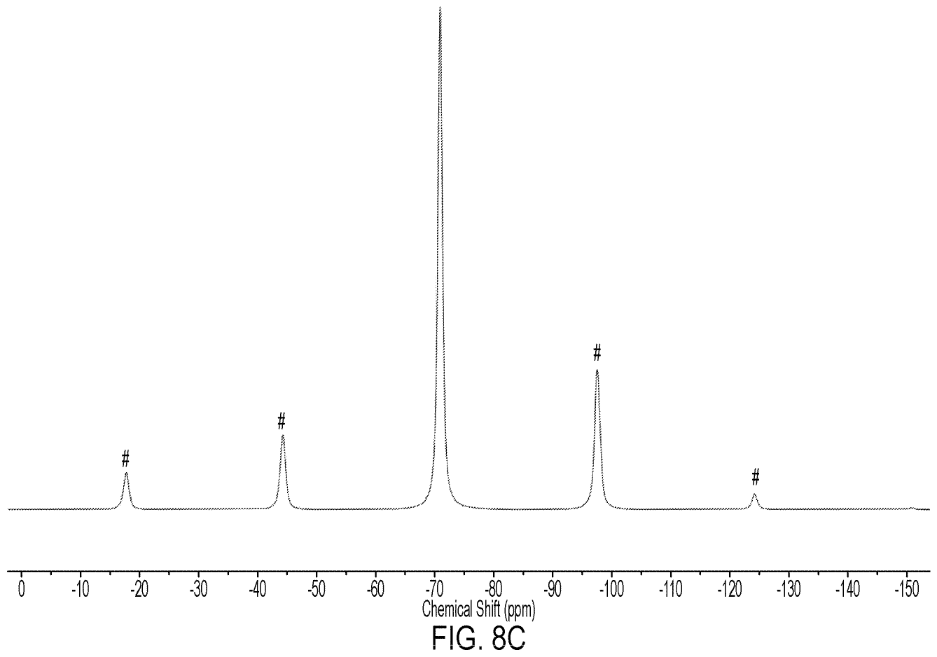

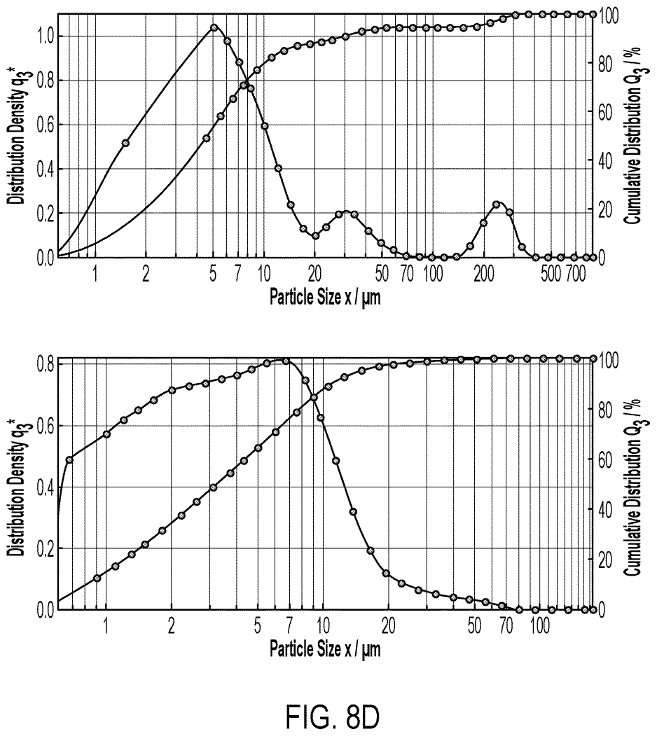

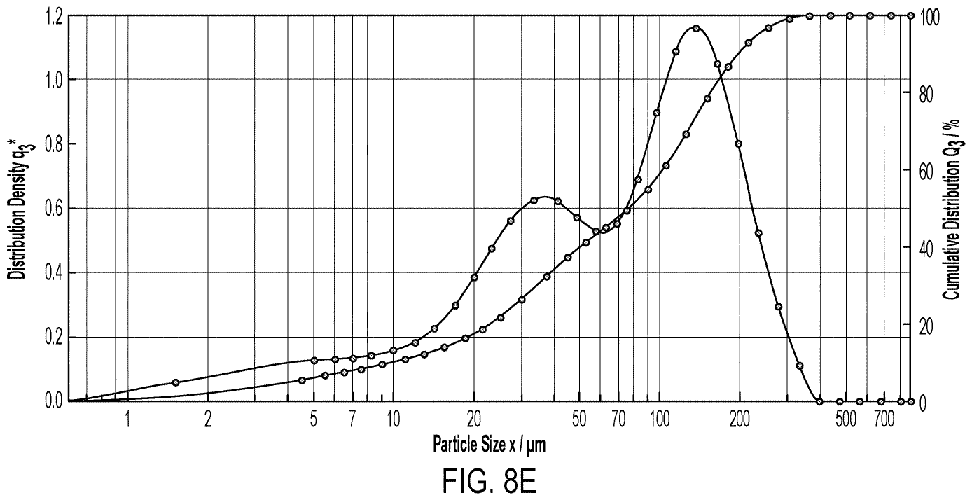

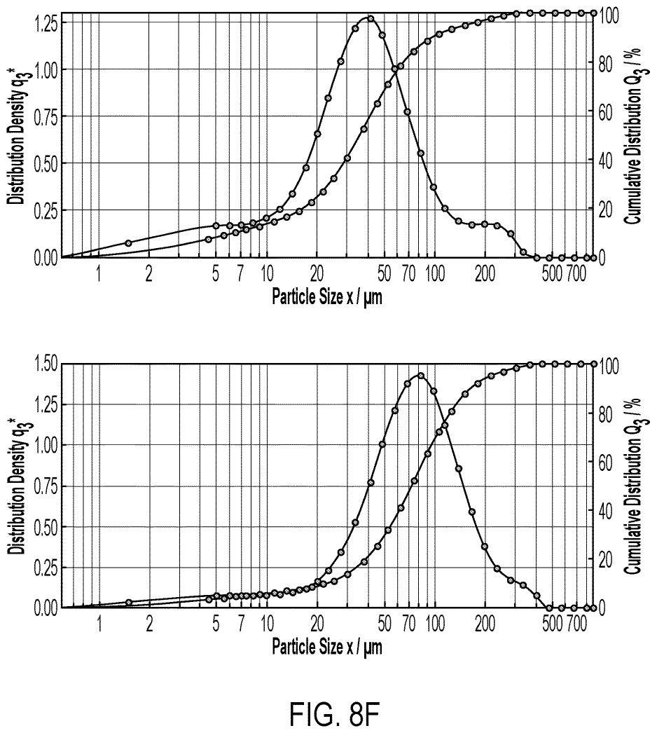

Powder X-Ray Diffraction (PXRD): The powder X-ray diffraction pattern and the peak list of Compound I Co-processed Form 1 with 10 wt % MCC is shown in FIG. 8A and the peaks are listed in the following table.

PXRD peak list for Compound I Co-processed Form 1 with 10 W % MCC

| Angle (°2 Θ ± | Relative |
|---|---|
| 0.2 °2 Θ) | Intensity (%) |
| 6.3 | 4 |
| 9.0 | 4 |
| 9.5 | 50 |
| 10.2 | 32 |
| 10.7 | 24 |
| 12.0 | 4 |
| 12.5 | 11 |
| 13.0 | 5 |
| 13.9 | 6 |
| 15.4 | 5 |
| 16.2 | 29 |
| 16.6 | 17 |
| 17.5 | 5 |
| 18.1 | 100 |
| 18.3 | 14 |
| 18.9 | 45 |
| 19.1 | 38 |
| 20.4 | 7 |
| 20.7 | 5 |
| 21.4 | 18 |
| 22.3 | 8 |
| 22.7 | 12 |
| 23.8 | 11 |
| 24.3 | 9 |
| 24.6 | 9 |
| 25.0 | 11 |
| 26.1 | 10 |
| 26.6 | 5 |
| 27.3 | 4 |
| 27.5 | 5 |
| 27.9 | 3 |
| 30.2 | 5 |
| 32.4 | 4 |
| 33.2 | 5 |
| 33.7 | 3 |

Solid-State Nuclear Magnetic Resonance (ssNMR):

The ¹³C and ¹⁹F ssNMR spectra for Compound I Co-processed Form 1 with 10 wt % MCC are shown in FIGS. 8B and 8C, and the corresponding peak lists are shown in the tables immediately below. The peaks marked by # in FIGS. 8B and 8C are spinning side bands.

TABLE
¹³C solid-state NMR peak list for Compound
I Co-processed Form 1 with 10 wt % MCC

| ¹³C Chemical | Relative |
|---|---|
| Shift (ppm) | Intensity (%) |
| 180.5 | 36 |
| 173.3 | 30 |
| 170.5 | 34 |
| 156.8 | 33 |
| 119.6 | 10 |
| 59.1 | 42 |
| 58.1 | 43 |
| 50.7 | 40 |
| 47.8 | 35 |
| 43.4 | 33 |
| 41.6 | 42 |
| 39.3 | 34 |
| 38.7 | 53 |
| 37.5 | 38 |
| 36.7 | 35 |
| 29.4 | 29 |
| 28.0 | 37 |
| 26.4 | 100 |

TABLE
¹⁹F solid-state NMR peak list for Compound
I Co-processed Form 1 with 10 wt % MCC.

| ¹⁹F Chemical | Relative |
|---|---|
| Shift (ppm) | Intensity (%) |
| −70.9 | 100 |

Particle Size Distribution Data of Compound I Form 1 and Compound I Co-Processed Form 1 with 10 wt % MCC

### PSD Method

These two types of material are measured using the generic method summarized below. This laser diffraction technique disperses the dry sample powder with compressed air. Three parameters are modified in this method to meet certain quality criteria, as defined in the method. The lens is changed to measure the appropriate range of particle sizes. The sample mass and sled speed are two parameters that are modified to achieve an optimal optical concentration.

Generally, additional sample mass is used in the coprocessed Form 1 measurement, however lens selection and sled speed can vary sample to sample.

| Instrument | Dry powder dispersion laser light | |
|---|---|---|
| | diffraction, e.g. Sympatec HELOS | |
| Disperser Type | RODOS/M, RODOS/L, OASISDRY, or | |
| | validated equivalent | |
| Feeder | ASPIROS, or validated equivalent | |
| Lens | Appropriate for particles in the range of | |
| | 0.5 μm to 350 μm, e.g. R4, or appropriate | |
| | for particles in the range of 0.5 μm to 875 | |
| | μm, or validated equivalent | |
| Evaluation Mode | LD | |
| Primary pressure | 1.0 | bar |
| Duration of | 10 | seconds |
| Reference Measurement | | |

| | |
|---|---|
| Trigger Condition | Optical concentration ≥1.0% |

### Preparation of Test Samples

Prepare samples in triplicate.

### Procedure

Invert and rotate vial to ensure material is in the bottom of the vial but is not compacted.

### Calculations

From triplicate determinations, report the mean values and % RSD of D[v,0.1], D[v,0.5], D[v,0.9], and D[4,3] to 2 decimal places.

### Results

A range of particle sizes have been measured for both API (Compound I Form 1) and co-processed Compound I Form 1 material. Several parameters can be used to describe a particle size distribution. Commonly accepted parameters are D[v,0.1], D[v,0.5], and D[v,0.9]. These numbers represent the volume based cumulative distribution. A D[v,0.9] represents 90% of the sample is smaller than the given value. D[v,0.5] and D[v,0.9] values are often used to describe particle size specifications. To quantitatively describe the particle sizes of both the API (Compound I Form 1) and coprocessed Compound I Form 1, the D[v,0.5] and D[v,0.9] values were utilized.

There has been a broad range of particle size observed for the API (Compound I Form 1). The D[v,0.5] ranged from 3 to 94 μm and the D[v,0.9] ranged from 17 to 250 μm. This is a wide spread of particle size values. In addition to the values, the shapes of the distributions also vary. The distributions are monomodal (one peak), bimodal (two peaks), and multimodal (more than two peaks). These particle size distributions are uncontrolled during its manufacture.

In FIGS. 8D-8E, the x-axis is a logarithmic scale of the particle size in microns. The left y-axis is the distribution density (arbitrary units), which is the most common way to view particle size distributions. The right y-axis is the cumulative distribution (percentage units) which directly correspond to the D[v] values referenced here. FIGS. 8D-8E show that the particle size distribution of Compound I, Form 1 in the absence of MCC is uncontrolled.

FIG. 8F shows particle size distributions for two different lots of Compound I, Form 1 co-processed with 10 wt % MCC with D[v,0.5]=36 μm and D[v,0.9]=98 μm (top) and D[v,0.5]=73 μm and D[v,0.9]=165 μm (bottom)—both with a monomodal particle size distribution. The Compound I co-processed Form 1 material (with 10 wt % MCC) particle size is well controlled during its manufacture. The particle size has been intentionally and controllably varied. The D[v,0.5] ranged from 31 to 81 μm and the D[v,0.9] ranged from 97 to 240 μm. The distributions are primarily monomodal and controllable which is advantageous in providing appropriate material for a drug product.

### Example 4

### Preparation of (2S,4R)-1-{(2S)-2-[(methoxycarbonyl)amino]-3,3-dimethylbutanoyl}-4-(trifluoromethyl)pyrrolidine-2-carboxylic acid (Compound 6′) (Step 1-8)

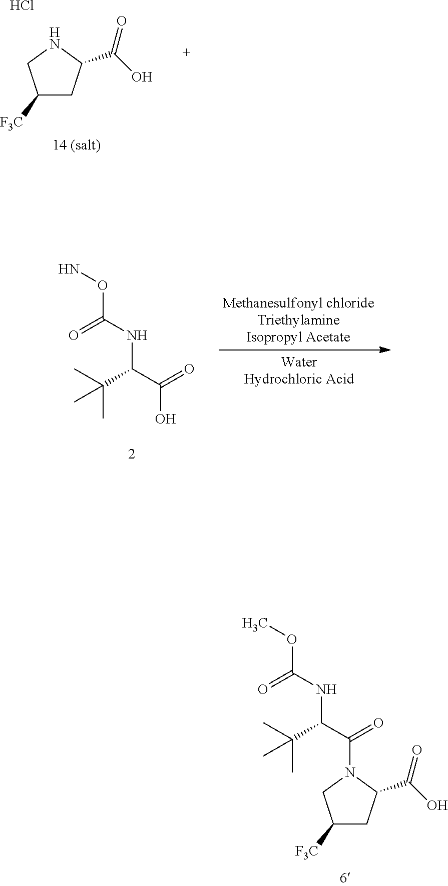 [SMILES: Cl ; O=C(O)[C@@H]1C[C@@H](C(F)(F)F)CN1 ; CC(C)(C)[C@H](NC(=O)ON)C(=O)O ; COC(=O)N[C@H](C(=O)N1C[C@H](C(F)(F)F)C[C@H]1C(=O)O)C(C)(C)C]

Compound 2 (1.41 g, 7.39 mmol, 1.09 equivalents), methanesulfonyl chloride (0.854 g, 7.46 mmol, 1.1 eq.) and isopropyl acetate (22 mL, 15 mL/g of Compound 14) are combined and stirred at 20 °C. Triethylamine (2.36 mL, 16.9 mmol, 2.5 eq.) is charged at a rate such that the reaction temperature does not exceed 25 °C, and the resulting mixture is stirred for 60 minutes. In a separate vessel, (2S,4R)-4-(trifluoromethyl) pyrrolidine-2-carboxylic acid hydrochloride, Compound 14 (1.5 g, 6.8 mmol, 1.0 eq.) and water (1.5 mL, 1 mL/g of Compound 14) are combined, stirred at 40 °C until solids dissolve, and charged to the reaction mixture above at 20 °C. Triethylamine (2.36 mL, 16.9 mmol, 2.5 eq.) and water (1.5 mL, 1 mL/g of Compound 14) are charged to the mixture. The resulting mixture is stirred for 20 hours at 20 °C. A sample is analyzed for reaction completion by UPLC (not more than 3% Compound 14). The reaction mixture is quenched by addition of water (12 mL, 8 mL/g of Compound 14), and the mixture is stirred not less than 10 min, then the layers are allowed to settle. The aqueous phase is removed and collected. The organic phase is extracted with water (12 mL, 8 mL/g of Compound 14). The combined aqueous phases are charged over 4 h to a stirring vessel containing water (23 mL, 15 mL/g of Compound 14), hydrochloric acid (12.2 Mol/L) in water (1.11 mL, 13.5 mmol, 2.0 eq.) and seed of Compound 6 free acid (117 mg, 0.33 mmol, 0.049 eq.) at 25 °C. The mixture is stirred for 16 hours at 25 °C. Solids are collected by filtration and rinsed with water (11 mL, 7 mL/g of Compound 14). The solids are dried at 60 °C in a vacuum oven to provide (2S,4R)-1-{(2S)-2-[(methoxycarbonyl) amino]-3,3-dimethylbutanoyl}-4-(trifluoromethyl)pyrrolidine-2-carboxylic acid, Compound 6 as a crystalline solid.

Compound 6 free acid, Note: in the ¹H, ¹⁹F and ¹³C NMR spectra, 2 sets of resonances were observed due to the presence of both E and Z amide bond rotamers in solution. Only the major rotamer resonances (89%) are listed here. ¹H NMR (400 MHz, DMSO-d₆, 298K): δ 12.76 (s, 1H), 7.24 (d, J=8.8 Hz, 1H), 4.41 (dd, J=9.0, 5.0 Hz, 1H), 4.17 (d, J=8.9 Hz, 1H), 3.94 (d, J=7.1 Hz, 2H), 3.53 (s, 3H), 3.45-3.27 (m, 1H), 2.43-2.28 (m, 1H), 2.26-2.11 (m, 1H), 0.97 (s, 9H). ¹⁹F NMR (376 MHz, DMSO-d₆, 298K): δ −70.15 (d, J=9.1 Hz). ¹³C NMR (101 MHz, DMSO-d₆, 298K): δ 172.46, 169.90, 156.95, 126.94 (q, J=278.1 Hz), 58.84, 57.88, 51.53, 46.36, 41.07 (q, J=27.9 Hz), 34.51, 27.75, 26.15. HRMS: (ESI+) Calcd for C14H22F3N2O5+: 355.1475, Found: 355.1478 (mass deviation+0.8 ppm).

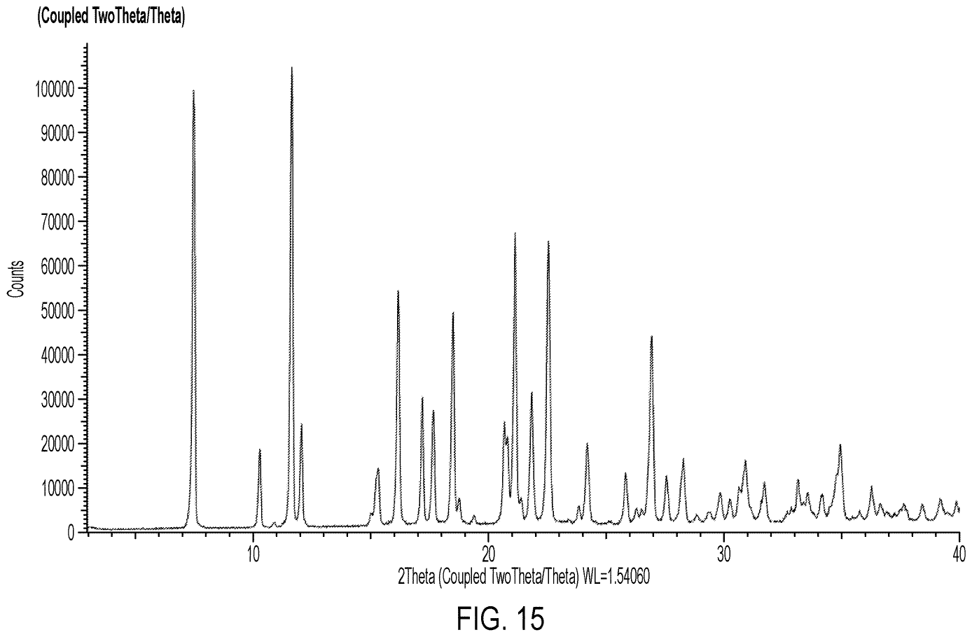

PXRD of crystalline Compound 6 (free acid form) was determined and is provided in FIG. 15 in which the following peaks were observed.

| Angle | |
|---|---|
| (°2 Θ ± | Relative |
| 0.2 °2 Θ) | Intensity |
| 7.5 | 92 |
| 10.3 | 17 |
| 10.9 | 1 |
| 11.4 | 3 |
| 11.7 | 100 |
| 12.1 | 23 |
| 15.1 | 3 |
| 15.3 | 13 |
| 16.2 | 53 |
| 17.2 | 28 |
| 17.7 | 25 |
| 18.5 | 48 |
| 18.8 | 6 |
| 19.4 | 2 |
| 20.7 | 23 |
| 20.8 | 19 |
| 21.2 | 69 |
| 21.4 | 6 |
| 21.9 | 30 |
| 22.6 | 64 |
| 23.4 | 1 |
| 23.9 | 4 |
| 24.2 | 19 |
| 25.9 | 12 |
| 26.3 | 4 |
| 26.5 | 3 |
| 27.0 | 44 |
| 27.6 | 11 |
| 28.3 | 14 |
| 28.9 | 2 |
| 29.4 | 2 |
| 29.9 | 7 |
| 30.3 | 5 |
| 30.7 | 6 |
| 30.9 | 13 |
| 31.1 | 3 |
| 31.6 | 6 |
| 31.8 | 10 |
| 32.7 | 3 |
| 32.9 | 4 |
| 33.2 | 11 |
| 33.4 | 4 |
| 33.6 | 7 |
| 34.2 | 6 |
| 34.5 | 4 |
| 34.8 | 11 |
| 35.0 | 19 |
| 35.8 | 2 |
| 36.3 | 9 |
| 36.7 | 4 |
| 37.5 | 3 |
| 37.7 | 4 |
| 38.5 | 4 |
| 39.2 | 5 |
| 39.9 | 4 |

### Example 5

### Preparation of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate, Compound 8

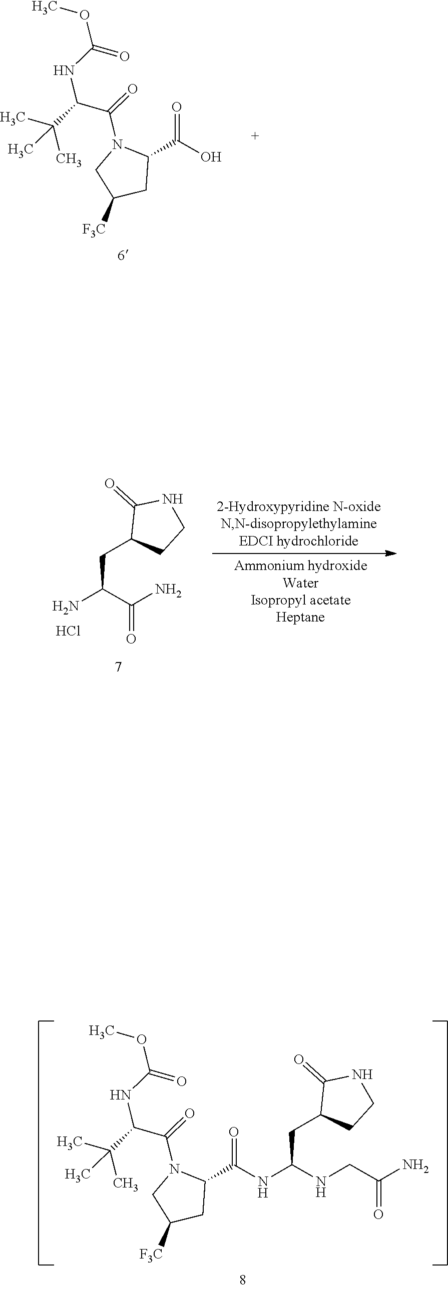

Compound 6′ (25.4 g, 69.7 mmol, 1.0 equivalents) and methyl ethyl ketone (MEK, 200 mL, 8 L/kg Compound 6′) are combined and stirred at 25 °C. 2-Hydroxypyridine N-oxide (7.91 g, 69.7 mmol, 1.0 eq.) and triethylamine (17.6 g, 24.3 mL, 0.174 mol, 2.50 eq.) are added, and the resulting solution is stirred for 5 min. Compound 7 (17.2 g, 80.2 mmol, 1.15 eq.) and 1-(3-dimethylaminopropyl)-3-ethyl-carbodiimide hydrochloride (EDCI hydrochloride, 20.1 g, 0.105 mol, 1.50 eq.) are charged, rinse with water (3.8 mL, 0.15 L/Kg Compound 6′) and methyl ethyl ketone (50 mL, 2 L/kg Compound 6′), and stirring is maintained for 16 h at 25 °C. The reaction is sampled (target of not more than 1% Compound 6′). If the reaction is not complete, the mixture is stirred for additional time. The reaction is quenched by the addition of aqueous NaCl (100 mL of a 14 wt % brine solution, 4.0 L/kg of Compound 6′), and stirring is maintained for 30 min. Stirring is stopped and the layers allowed to settle. The lower aqueous phase is removed, and the organic phase is washed with a second portion of aqueous NaCl (100 mL of a 14 wt % brine solution), following the same protocol. Both aqueous phases are combined and extracted with methyl ethyl ketone (125 mL, 5 L/kg Compound 6′), stirred for 5 min and phases were separated. The organic phase is combined with previous organic layer and aqueous phase was extracted with a second portion of methyl ethyl ketone (125 mL, 5 L/kg Compound 6′), following the same protocol. All organic phases are combined and then concentrated by vacuum distillation at 0.3 bar (internal temperature of reaction mixture ~30 °C) up to reached ~5 L/kg. Then isopropyl acetate (200 mL, 8 L/kg Compound 6′) was added to the mixture and distillation continue to reach ~5 L/kg reaction volume. A second addition of isopropyl acetate (200 mL, 8 L/kg Compound 6′) is added, and the distillation process was repeated following the same protocol, ending the distillation at 5 L/kg. Isopropyl acetate (125 mL, 5 L/kg Compound 6′) was added and stirred at 25 °C. A sample is analyzed for water content (Karl-Fischer) with a target of not more than 0.2 wt % water and not more than 1 wt % of MEK. The resulting organic solution of Compound 8 is used in Step 3 without further purification.

Product Characterization of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate, Compound 8: A sample of the solution containing Compound 8 was concentrated to dryness and purified by column chromatography for characterization purpose using a solvent mixture of dichloromethane:methanol (90:10) and RediSep Gold® Silica Gel Disposable Flash Columns.

Note: in the ¹H and ¹³C NMR spectra, the sample contains two minor rotamers due to the N5-C9 and N15-C16 amide bonds, making up approximately 9% and 7% of the sample, respectively. Where relevant, the less abundant rotamer signals are designated as “minor.” ¹H NMR (600 MHz, DMSO-d₆, 298K): d 5 8.47 (d, J=7.88 Hz, minor), 8.28 (d, J=8.96 Hz, 1H), 7.62 (s, minor), 7.54 (s, 1H), 7.42 (s, minor), 7.33 (s, 1H), 7.25 (d, J=8.96 Hz, 1H), 7.04 (s, 1H), 7.01 (s, minor), 6.83 (br, minor) 6.78 (d, J=9.12 Hz, minor), 4.92 (br t, J=5.93 Hz, minor), 4.51 (dd, J=8.26, 5.29 Hz), 4.26 (ddd, J=12.16, 8.86, 3.55 Hz, 1H), 4.15 (d, J=8.83 Hz, 1H), 4.10 (d, J=9.08 Hz, minor), 4.07 (br, minor) 3.89-3.97 (om, 2H), 3.85 (br, minor), 3.62 (m, minor), 3.52 (s, 3H), 3.49 (s, minor), 3.37 (m, 1H), 3.25 (m, minor), 3.17 (m, minor), 3.12 (t, J=9.15 Hz, 1H), 3.03 (td, J=9.85, 7.23 Hz, 1H), 2.43 (m, 1H), 2.33 (m, minor), 2.24 (m, 1H), 2.09-2.16 (om, 2H), 2.01 (m, minor), 1.94 (ddd, J=13.70, 12.10, 3.57 Hz, 1H), 1.70 (m, minor), 1.62 (m, 1H), 1.54 (m, minor), 1.49 (ddd, J=13.74, 11.96, 3.66 Hz, 1H), 0.95 (s, 9H), 0.89 (s, minor). ¹³C NMR (151 MHz, DMSO-d₆, 298K): d 178.7 (minor), 178.5, 173.4 (minor), 173.3, 170.8, 170.4 (minor), 169.6, 169.2 (minor), 156.9, 156.0 (minor), 127.0 (1JCF=278.4 Hz), 58.8, 58.6, 58.3 (minor), 51.4, 51.3 (minor), 51.2 (minor), 50.3, 46.6, 45.5 (minor), 41.1 (2JCF=28.0 Hz), 39.9, 39.4 (minor, ov), 39.2 (ov), 38.6 (2JCF=28.5 Hz, minor), 37.6 (minor), 37.1, 35.2 (minor), 34.4, 34.0, 33.5 (minor), 30.7 (minor), 28.2, 27.4 (minor), 27.3, 26.1, 26.0 (minor). ¹⁹F NMR (376 MHz, DMSO-d₆, 298K): d −70.07, −70.43 (minor), −70.48 (minor). om=overlapped multiplet, ov=overlapped with solvent, br=broad signal HRMS: (ESI+) Calcd for C₂₁H₃₃F₃N₅O₆⁺: 508.2377, Found: 508.2377 (mass deviation −0.1 ppm)

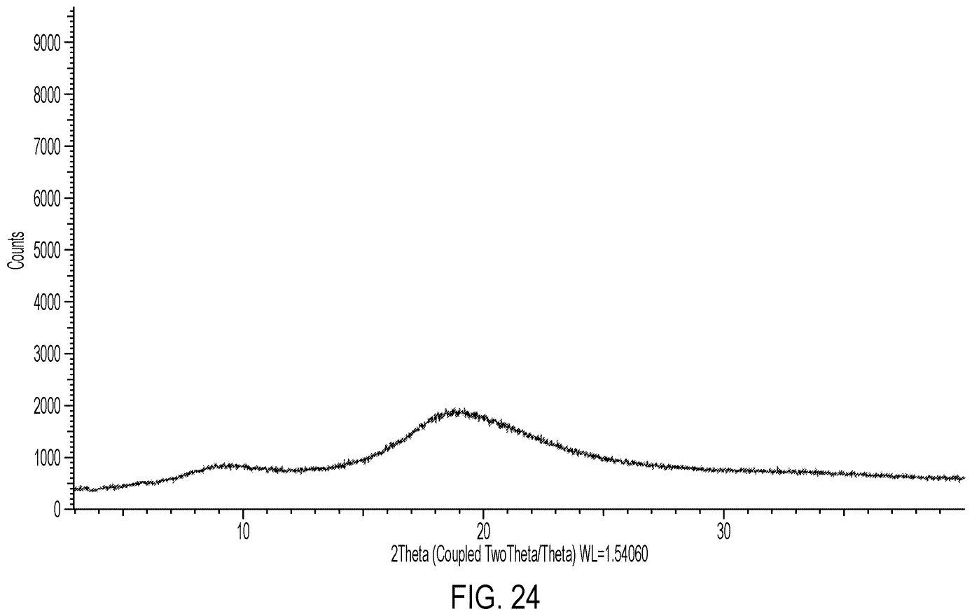

Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate (amorphous form) can be characterized by PXRD and the PXRD pattern is as shown in FIG. 24.

EXAMPLES 5A to 5F provide different co-crystal solid forms of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl) pyrrolidin-1-yl]-3,3-dimethyl-1-oxobutan-2-yl}carbamate with the specified carboxylic acid containing compounds.

### Example 5A

### Preparation of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate tartrate co-crystal

To a 100 mL vessel with overhead stirring was charged a solution of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate Compound 8 in 2-butanone prepared as described above (96 mL solution, 81.6 g with 8.25 wt %, 6.8 g of Compound 8). The solution is concentrated by vacuum distillation (100-200 mbar and (internal temperature 40 °C)) to a volume of ~50 mL (20 mL, 6 L/kg), 2-butanone (20 mL, 3 L/kg) is added, and the solution is concentrated by vacuum distillation to a volume of 50 mL (20 mL, 6 L/kg). A sample is analyzed for water content (Karl-Fisher) with a target of not more than 1 wt % water. Reaction mixture was heated at 45 °C, D-(−)-tartaric acid (4.08 g, 27.2 mmol, 2 equiv.) was charged and stirred at 75 °C to get everything in solution. The solution is cooled to 60 °C at a rate of 1 °C/min then the mixture was seeded at 60 °C using Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate tartrate (30 mg, 0.046 mmol, 0.005 equiv), hold for 1 hour, cooled down to 5 °C at rate 0.3 °C/min. Solids are collected by filtration, rinsed with 2-butanone (20 mL, 3 mL/kg), and dried in a vacuum oven at 50 °C for 8 hours providing 7.76 g of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate tartrate (co-crystal).

Note: This procedure can be done without distillation by using amorphous Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate in 2-butanone (6 L 2-butanone/kg of Compound 8).

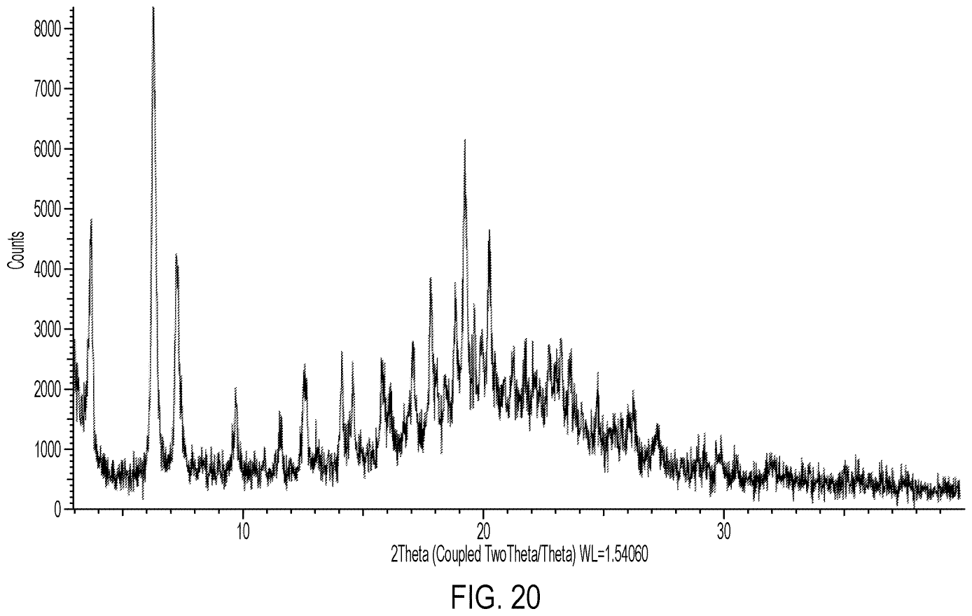

PXRD pattern of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate tartrate co-crystal was determined and is shown in FIG. 20 in which the following peaks were observed.

| Angle (°2 Θ ± | Relative |
|---|---|
| 0.2 °2 Θ) | Intensity |
| 3.7 | 37 |
| 6.3 | 100 |
| 7.3 | 35 |
| 9.7 | 18 |
| 11.6 | 11 |
| 12.6 | 19 |
| 14.1 | 25 |
| 14.6 | 20 |
| 15.8 | 13 |
| 16.2 | 12 |
| 17.1 | 11 |
| 17.9 | 28 |
| 18.5 | 4 |
| 18.9 | 22 |
| 19.2 | 59 |
| 19.6 | 32 |
| 20.0 | 18 |
| 20.3 | 35 |
| 20.9 | 8 |
| 21.2 | 8 |
| 21.7 | 18 |
| 22.8 | 11 |
| 23.2 | 21 |
| 23.6 | 15 |
| 24.7 | 14 |
| 26.1 | 9 |
| 26.2 | 16 |
| 29.8 | 7 |

### Example 5B

### Preparation of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate maleate co-crystal

To a 100 mL vessel with overhead stirring was charged Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl) pyrrolidin-1-yl]-3,3-dimethyl-1-oxobutan-2-yl}carbamate, Compound 8 (5 g, 9.86 mmol) in 2-butanone (5 L/kg) and n-heptane (1.7 L/kg). Reaction mixture was heated at 70 °C, maleic acid (1.45 g, 12.4 mmol, 2 equiv.) was charged and stirred at 70 °C for 6 hours. The solution is cooled to 40 °C at a rate of 0.3 °C/min. The mixture was seeded at 40 °C using Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl) pyrrolidin-1-yl]-3,3-dimethyl-1-oxobutan-2-yl}carbamate maleate (20 mg, 0.005 wt %), hold for 1 hour, cooled down to 5 °C at a rate of 0.3 °C/min. Solids are collected by filtration, rinsed with a mixture 4:1 2-butanone:n-heptane (20 mL, 2 mL/kg), and dried in a vacuum oven at 50 °C for 17 hours providing 1.94 g of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl) pyrrolidin-1-yl]-3,3-dimethyl-1-oxobutan-2-yl}carbamate maleate co-crystal.

This co-crystal can also be formed in 2-butanone or isopropyl acetate.

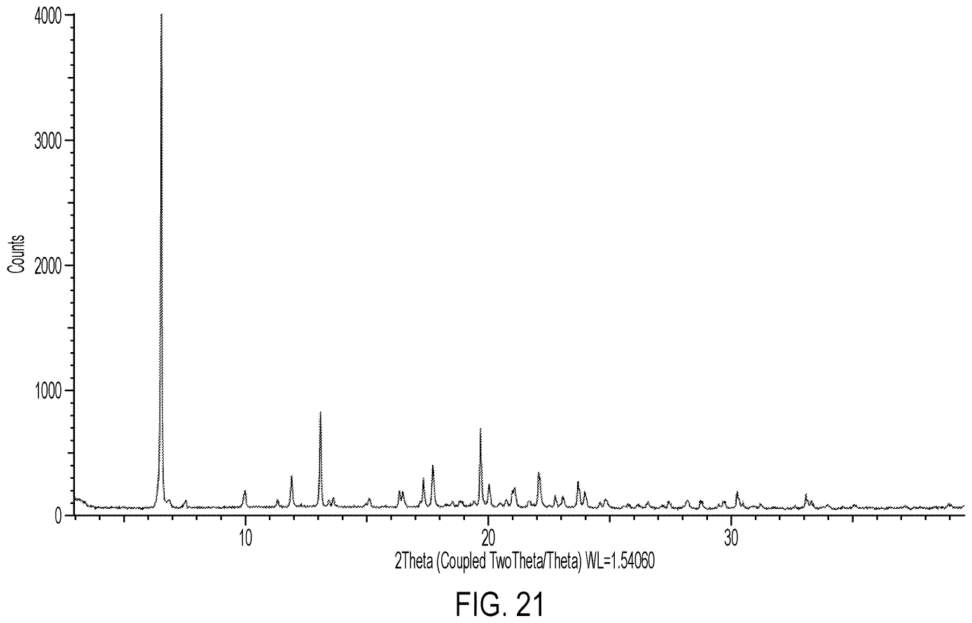

The PXRD pattern of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl) pyrrolidin-1-yl]-3,3-dimethyl-1-oxobutan-2-yl}carbamate maleate co-crystal was determined and is shown in FIG. 21 in which the peaks in the following table were observed.

| Angle (°2 Θ ± | Relative |
|---|---|
| 0.2 °2 Θ) | Intensity |
| 6.6 | 100 |
| 6.9 | 1 |
| 7.5 | 1 |
| 10.0 | 3 |
| 11.3 | 1 |
| 11.9 | 7 |
| 13.1 | 21 |
| 13.4 | 1 |
| 13.6 | 2 |
| 15.1 | 2 |
| 16.4 | 3 |
| 16.5 | 3 |
| 17.2 | 1 |
| 17.3 | 6 |
| 17.7 | 9 |
| 18.5 | 1 |
| 18.9 | 1 |
| 19.4 | 1 |
| 19.7 | 18 |
| 20.0 | 5 |
| 20.5 | 1 |
| 20.7 | 1 |
| 21.1 | 4 |
| 21.7 | 1 |
| 22.1 | 7 |
| 22.8 | 3 |
| 23.1 | 3 |
| 23.7 | 6 |
| 24.0 | 4 |
| 24.6 | 1 |
| 24.8 | 2 |
| 25.8 | 1 |
| 26.2 | 1 |
| 26.6 | 1 |
| 27.2 | 1 |
| 27.4 | 2 |
| 28.2 | 2 |
| 28.7 | 2 |
| 29.5 | 1 |
| 29.7 | 2 |
| 30.3 | 4 |
| 30.5 | 1 |
| 31.2 | 1 |
| 32.6 | 1 |
| 33.1 | 2 |
| 33.3 | 2 |
| 34.0 | 1 |
| 35.1 | 1 |

### Example 5C

### Preparation of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate succinate co-crystal

To a 100 mL vessel with overhead stirring was charged Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl) pyrrolidin-1-yl]-3,3-dimethyl-1-oxobutan-2-yl}carbamate, Compound 8 (5 g, 9.86 mmol) in tetrahydrofuran (15 L/kg). The reaction mixture was heated at 60 °C, succinic acid (1.16 g, 9.8 mmol, 1 equiv.) was charged and stirred at 60 °C for 1 hour. Solution is cooled to 50 °C at a rate of 1 °C/min then n-heptane (10 L/kg) was added via syringe over 6 hours, held for 1 hour, then cooled down to 5 °C at a rate of 0.3 °C/min. Solids are collected by filtration, rinsed with n-heptane (50 mL, 10 L/kg), and dried in a vacuum oven at 50 °C for 17 hours providing 4.26 g of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate succinate (co-crystal, partially crystalline).

Crystallization: Charge Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate succinate (co-crystal, partially crystalline) from above and tetrahydrofuran (0.5 L/kg) was added until solids are nearly dissolved (thick paste). The slurry is allowed to stand at 25 °C for 2 weeks until precipitates appeared. Solids are dried at 25 °C to maintain crystallinity.

Note: crystallization can also occur using acetone, 2-butanone, or 1:1 mixture of methyl tert-butyl ether:ethyl acetate as solvent instead of tetrahydrofuran.

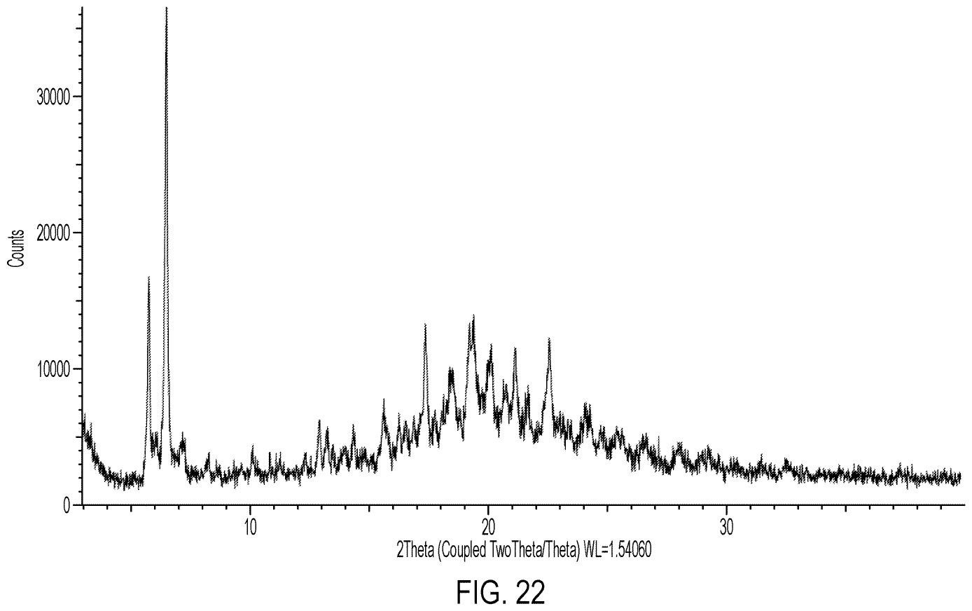

PXRD pattern of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate succinate co-crystal was determined and is shown in FIG. 22 in which the peaks in the following table were observed.

| Angle (°2 Θ ± | Relative |
|---|---|
| 0.2 °2 Θ) | Intensity |
| 5.2 | 3 |
| 5.8 | 43 |
| 6.5 | 100 |
| 7.1 | 8 |
| 8.2 | 4 |
| 10.1 | 2 |
| 11.2 | 4 |
| 12.3 | 4 |
| 12.9 | 11 |
| 13.2 | 8 |
| 13.5 | 5 |
| 14.0 | 5 |
| 14.3 | 10 |
| 15.7 | 9 |
| 16.3 | 4 |
| 16.6 | 6 |
| 16.9 | 6 |
| 17.4 | 25 |
| 17.8 | 7 |
| 18.1 | 11 |
| 18.5 | 12 |
| 19.3 | 25 |
| 20.1 | 14 |
| 20.7 | 12 |
| 21.1 | 20 |
| 21.6 | 5 |
| 22.5 | 23 |
| 24.1 | 4 |
| 24.8 | 3 |
| 25.5 | 6 |
| 26.5 | 2 |
| 28.0 | 4 |
| 29.0 | 4 |
| 29.2 | 7 |
| 29.6 | 2 |
| 29.7 | 4 |
| 30.2 | 5 |
| 30.3 | 3 |
| 30.4 | 3 |
| 30.3 | 3 |
| 31.0 | 4 |
| 31.5 | 6 |
| 32.5 | 4 |
| 33.3 | 4 |
| 37.3 | 4 |

### Example 5D

### Preparation of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate fumarate co-crystal

To a 100 mL vessel with overhead stirring was charged Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl) pyrrolidin-1-yl]-3,3-dimethyl-1-oxobutan-2-yl}carbamate, Compound 8 (5 g, 9.86 mmol) in tetrahydrofuran (25 L/kg). Reaction mixture was heated at 60 °C, fumaric acid (1.14 g, 9.9 mmol, 1 equiv.) was charged and stirred at 60 °C for 16 hour. If it is still a slurry, 4 L/kg of water is added at 60 °C. Solution is cooled to 5 °C over 12 hours. Solution is distilled twice under vacuum at 50 °C until concentration is 6 L/kg of Compound 8. Then n-heptane (6 L/kg) was added over 12 hours, held for 1 hour at 45-50 °C, then cooled down to 5 °C at a rate of 0.3 °C/min. Solids are collected by filtration, rinsed with n-heptane (2 L/kg), and dried in a vacuum oven at 50 °C for 16 hours providing 1.35 g of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl) pyrrolidin-1-yl]-3,3-dimethyl-1-oxobutan-2-yl}carbamate fumarate co-crystal.

Crystallization: Charge Compound 8 succinate from above and tetrahydrofuran (0.5 L/kg) was added until solids are nearly dissolved (thick paste). Slurry is allowed to stand at 25 °C for 2 weeks until precipitates appeared. Solids are dried at 25 °C to maintain crystallinity.

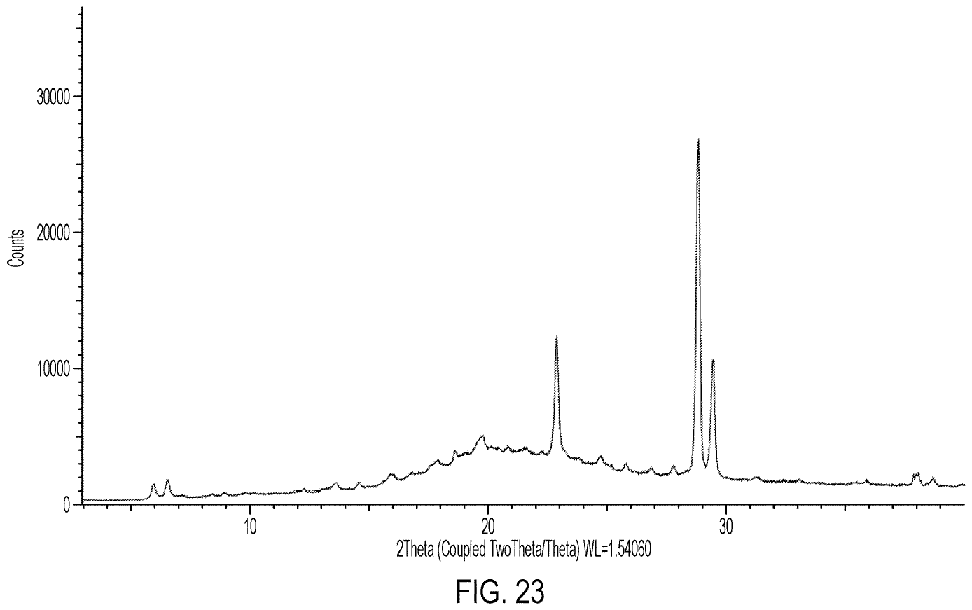

PXRD pattern of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate fumarate co-crystal was determined and is shown in FIG. 23 in which the peaks in the following table were observed.

| Angle (°2 Θ ± | Relative |
|---|---|
| 0.2 °2 Θ) | Intensity |
| 6.0 | 4 |
| 6.5 | 5 |
| 7.2 | 1 |
| 8.4 | 1 |
| 9.0 | 1 |
| 12.3 | 1 |
| 13.6 | 2 |
| 14.6 | 2 |
| 15.6 | 1 |
| 15.9 | 3 |
| 16.0 | 3 |
| 18.6 | 8 |
| 19.7 | 11 |
| 19.8 | 12 |
| 20.4 | 7 |
| 20.8 | 7 |
| 22.3 | 5 |
| 22.9 | 40 |
| 24.7 | 5 |
| 25.2 | 2 |
| 25.8 | 3 |
| 26.8 | 1 |
| 27.8 | 3 |
| 28.8 | 100 |
| 29.4 | 38 |
| 31.3 | 1 |
| 33.1 | 1 |
| 35.5 | 1 |
| 35.9 | 2 |
| 37.9 | 4 |
| 38.0 | 4 |
| 38.1 | 4 |
| 38.7 | 3 |

### Example 5E

### Preparation of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate 4-hydroxybenzoic acid co-crystal

Co-crystals with 4-hydroxybenzoic acid as conformer can be prepared in a manner analogous to Examples 5A-5D and were obtained using a mixture of 1:1 isopropyl acetate:heptane as solvent and using 3 or 4 equivalents of 4-hydroxybenzoic acid.

### Example 5F

### Preparation of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate 2,5-dihydroxybenzoic acid co-crystal

Co-crystals with 2,5-dihydroxybenzoic acid (gentisic) as conformer can be prepared in a manner analogous to Examples 5A-5D and were obtained using a mixture of 1:1 isopropyl acetate:heptane as solvent with 2,5-dihydroxybenzoic acid (gentisic).

### Example 6

### Preparation of Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate methoxycyclopentane solvate, Compound I CPME solvate

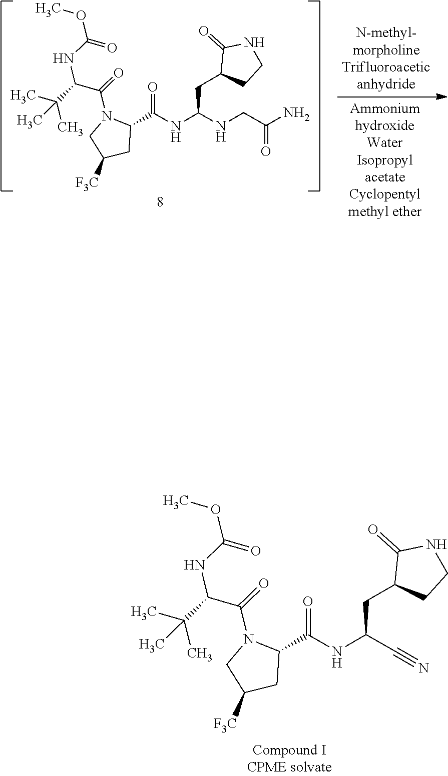

The isopropyl acetate solution of Compound 8 prepared above (assumed quantitate conversion, 69.7 mmol, 1.0 equivalents) is combined with N-methylmorpholine (36.7 g, 40 mL, 0.36 mol, 5.2 equivalents) and stirred at 5-10 °C. Trifluoroacetic anhydride (38.1 g, 25.5 mL, 0.18 mol, 2.6 equivalents) is charged over 30-60 minutes, maintaining the reaction temperature at not more than 15 °C. The resulting mixture is stirred for 1 h. A sample is analyzed for reaction completion (not more than 0.5% Compound 8). If the reaction is not complete, maintain stirring for another 60 min, and charge additional N-methylmorpholine and trifluoroacetic anhydride (maintaining a 2:1 ratio) if needed. The reaction is quenched by addition of aq. ammonium hydroxide (28 wt %) (10.7 mL, 76 mmol, 1.1 equivalents) in water (in 74.1 mL water, 3.0 L/kg of Compound 6 free acid from previous step) or (84.8 mL of a 3.5% wt % ammonium hydroxide solution, 3.34 L/kg of Compound 6 free acid from previous step), stirring is maintained for 30 min, then stopped and the layers allowed to settle. The aqueous phase is removed, and the organic phase is sampled (target of not more than 0.1% methyl ((S)-1-((2S,4R)-2-(((S)-1-cyano-2-((S)-2-oxopyrrolidin-3-yl)ethyl)carbamoyl)-4-(trifluoromethyl)pyrrolidin-1-yl)-3,3-dimethyl-1-oxobutan-2-yl)(2,2,2-trifluoroacetyl)carbamate). If target is not achieved, a second basic wash is required (1.1 equiv. NH₄OH 28 wt % mixed with and 74.1 mL of water (3.0 L/kg), following the same protocol. The organic phase is washed with water (74.1 mL, 3.0 L/kg of Compound 6 free acid from previous step). The organic phase is then concentrated by vacuum distillation (0.3 bar and (internal temperature ~30 °C)) to a volume of 200 mL (8 L/kg of Compound 6 free acid from previous step). Cyclopentyl methyl ether (127 mL, 5.0 L/kg of Compound 6 free acid from previous step) is added, and the solution is concentrated by vacuum distillation to a volume of 200 mL (8 L/kg of Compound 6 free acid from previous step). This cyclopentyl methyl ether addition and distillation process is repeated three more times. A sample is analyzed for water content (Karl-Fischer) with a target of not more than 0.2 wt % water and not more than 1% isopropyl acetate content. If target values were not reached repeat the distillation process following the same protocol. The resulting solution/slurry is stirred at 40 °C for 30-60 minutes and cooled to 10 °C at a rate of 0.1 K/min and stirred at 10 °C for at least 1 h. Solids are collected by filtration, rinsed with 2.5 L/kg and 1 L/kg of CPME, and dried in a vacuum oven at 70 °C for 12 h providing 21.1 g of Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate methoxycyclopentane (CPME) solvate.

### Product Characterization:

Note: The sample is a CPME solvate with API:CPME molar ratio of approximately 5:2. The API contains two minor rotamers due to the N14-C18 and N24-C25 amide bonds, making up approximately 6.7% and 9.2%, respectively. Where relevant, the less abundant rotamer signals are designated as “minor.”

¹H NMR (600 MHz, DMSO-d₆, 298K): δ 9.08 (d, J=7.04 Hz, minor), 9.00 (d, J=8.59 Hz, 1H), 7.70 (s, minor), 7.64 (s, 1H), 7.24 (d, J=8.75 Hz, 1H), 6.91 (d, J=9.08 Hz, minor), 6.83 (br, minor), 4.94 (ddd, J=10.91, 8.58, 5.02 Hz, 1H), 4.89 (q, J=7.53 Hz, minor), 4.80 (dd, J=8.37, 2.93 Hz, minor), 4.37 (dd, J=8.19, 6.19 Hz, 1H), 4.15 (d, J=8.74 Hz, 1H), 4.07 (br, minor), 4.04 (d, J=8.99 Hz, minor), 3.96 (m, 2H), 3.86 (br, minor), 3.74 (m, CPME, 1H), 3.66 (d, J=7.07 Hz, minor), 3.52 (s, 3H), 3.50 (s, minor), 3.40 (m, 1H), 3.27-3.32 (m, overlapped with residual water, minor), 3.12-3.19 (om, minor), 3.15 (s, CPME, 3H), 3.13 (om, 1H), 3.04 (td, J=9.35, 7.07 Hz, 1H), 2.45 (m, 1H), 2.30 (m, 1H), 2.16 (ddd, J=13.60, 11.03, 4.47 Hz, 1H), 2.04-2.13 (om, 2H), 1.84 (m, minor), 1.64-1.75 (om, 2H), 1.51-1.65 (om, CPME, 6H), 1.46 (m, CPME, 2H), 0.94 (s, 9H), 0.89 (s, minor). ¹³C NMR (151 MHz, DMSO-d₆, 298K): d 177.5 (minor), 177.4, 170.8, 170.4 (minor), 169.8, 169.4 (minor), 156.9, 156.3 (minor), 127.1 (1JCF=277.4 Hz, minor), 126.9 (1JCF=277.8 Hz), 119.5, 119.2 (minor), 81.9 (CPME), 58.9, 58.5, 58.4 (minor), 55.5 (CPME), 51.4, 51.3 (minor), 46.7, 45.3 (minor), 41.2 (2JCF=27.9 Hz), 39.2 (overlapped with solvent), 38.6 (2JCF=28.2 Hz, minor), 37.7, 37.3 (minor), 36.6, 35.1 (minor), 34.3, 34.1, 33.2 (minor), 31.3 (CPME), 30.4 (minor), 28.1, 27.3 (minor), 26.8, 26.0, 25.9 (minor), 23.0 (CPME). ¹⁹F NMR (376 MHz, DMSO-d₆, 298K): δ −70.71, −70.99 (minor), −71.12 (minor). om=overlapped multiplet, br=broad signal. HRMS: (ESI+) Calcd for C₂₁H₃₁O₅N₅F₃ 490.2272 Da, found 490.2272 Da.

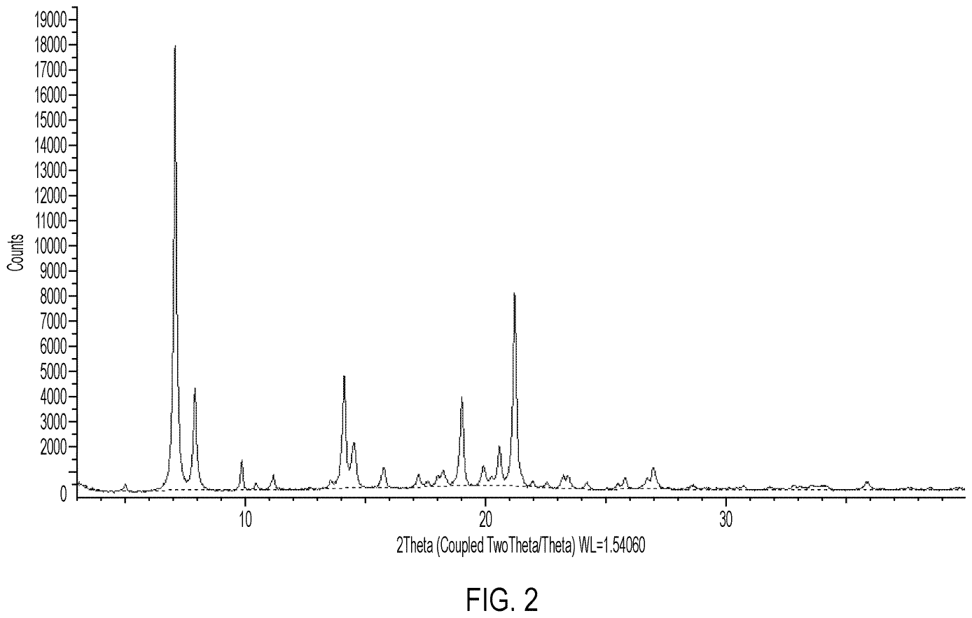

PXRD was determined and is shown in FIG. 2.

### Example 7

### Preparation of Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate propan-2-yl acetate solvate, Compound I IPAc solvate

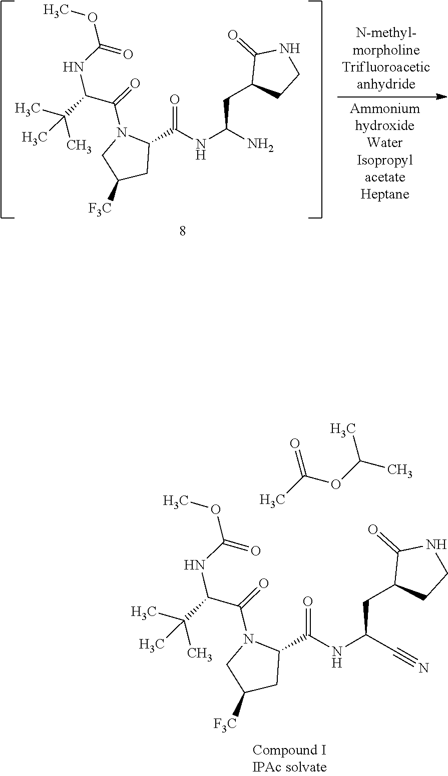

The isopropyl acetate solution of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate, Compound 8 prepared on step 3 (assumed quantitative conversion of 8.2 g Compound 6 free acid, 22.4 mmol, 1.0 eq.) is combined with N-methylmorpholine (11.8 g, 12.8 mL, 0.12 mol, 5.2 eq.) and stirred at 5-10 °C. Trifluoroacetic anhydride (12.2 g, 8.2 mL, 0.06 mol, 2.6 eq.) is charged over 30-60 min, maintaining the reaction temperature at not more than 15 °C. The resulting mixture is stirred for 1 h. A sample is analyzed for reaction completion (not more than 0.5% Compound 8). If the reaction is not complete, maintain stirring for another 60 min, and charge additional N-methylmorpholine and trifluoroacetic anhydride (maintaining a 2:1 ratio) if needed. The reaction is quenched by addition of aq. ammonium hydroxide (28 wt %) (3.4 mL, 24 mmol, 1.1 eq.) in water (in 23.8 mL water, 3.0 L/kg of Compound 6 free acid from step 3) or (27.3 mL of a 3.5% wt % ammonium hydroxide solution, 3.34 L/kg of Compound 6 free acid from step 3), stirring is maintained for 30 min, then stopped and the layers allowed to settle. The aqueous phase is removed, and the organic phase is sampled (target of not more than 0.1% methyl ((S)-1-((2S,4R)-2-(((S)-1-cyano-2-((S)-2-oxopyrrolidin-3-yl)ethyl)carbamoyl)-4-(trifluoromethyl)pyrrolidin-1-yl)-3,3-dimethyl-1-oxobutan-2-yl)(2,2,2-trifluoroacetyl)carbamate). If target is not achieved, a second basic wash is required (1.1 equiv. NH₄OH 28 wt % mixed with and 74.1 mL of water (3.0 L/kg)), following the same protocol. The organic phase is washed with water (23.7 mL, 3.0 L/kg of Compound 6 free acid from step 3). The organic phase is then concentrated by vacuum distillation (0.3 bar and (internal temperature ~30-40 °C)) to a volume of 63.4 mL (8 L/kg of Compound 6 free acid from step 3). Isopropyl acetate (63.4 mL, 8 L/kg of Compound 6 free acid from step 3) is added, and the solution is concentrated by vacuum distillation to a volume of 63.4 mL (8 L/kg of Compound 6 free acid from step 3). Isopropyl acetate (63.4 mL, 8 L/kg of Compound 6 free acid from step 3) is added, and the solution is concentrated by vacuum distillation to a volume of 63.4 mL (8 L/kg of Compound 6 free acid from step 3). A sample is analyzed for water content (Karl-Fischer) with a target of not more than 0.2 wt % water. If target failed water content then continue with the distillation process following the same protocol until met. This solution is stirred at 55 °C (internal temperature) and n-heptane (29.3 mL, 3.7 L/kg of Compound 6 free acid from step 3) is added in 30 min. Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate IPAc solvate seeds (0.21 g, 0.42 mmol, 2.5 wt % of Compound 6 free acid from step 3) are added. If seeds are dissolved add more heptane (0.37 L/Kg of Compound 6 free acid from step 3) and seed again. The internal temperature is held between 50-55 °C for 60 min. n-Heptane (69 mL, 8.7 L/kg of Compound 6 free acid from step 3) at 55 °C (internal temperature) in 8 h. This slurry is stirred at 55 °C for at least 30 minutes, cooled to 10 °C in 5 h, and stirred at 10 °C for at least 2 h. Solids are collected by filtration, rinsed with a pre-cooled mixture of n-heptane (1.2 L/Kg of Compound 6 free acid from step 3) and isopropyl acetate (0.8 L/Kg of Compound 6 free acid from step 3) at 10 °C twice, and dried in a vacuum oven at 50 °C for 12 hours providing 7.58 g of Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate, Isopropyl Acetate (IPAc) solvate.

### Product Characterization:

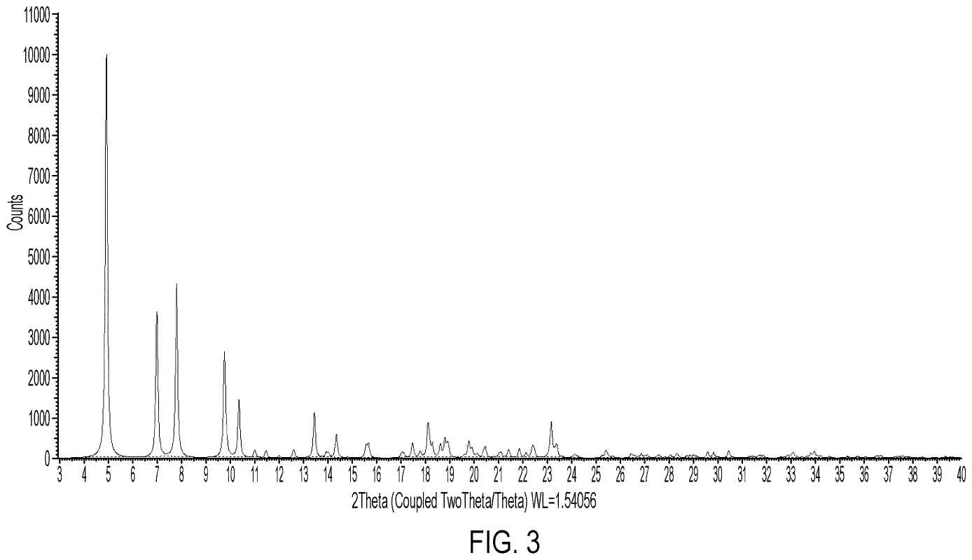

Note: The sample contains two minor rotamers due to the N14-C₁₈ and N24-C₂₅ amide bonds, making up approximately 8% and 14% of the sample, respectively. Isopropyl acetate (IPAc) is present as a solvate in the ratio of approximately 1.1:2 with respect to the API. Where relevant, the less abundant rotamer signals are designated as “minor.” ¹H NMR (600 MHz, DMSO-d₆, 298K): δ 9.09 (d, J=7.00 Hz, minor), 9.02 (d, J=8.59 Hz, 1H), 7.72 (s, minor), 7.66 (s, 1H), 7.27 (d, J=8.67 Hz, 1H), 6.94 (d, J=9.07 Hz, minor), 6.85 (br, minor), 4.95 (ddd, J=10.99, 8.57, 4.98 Hz, 1H), 4.86 (m, IPAc, 1H), 4.80 (dd, J=8.58, 3.10 Hz, minor), 4.36 (t, J=7.29 Hz, 1H), 4.15 (d, J=8.74 Hz, 1H), 4.07 (br, minor), 4.04 (d, J=9.12 Hz, minor), 3.96 (m, 2H), 3.86 (br, minor), 3.66 (d, J=7.13 Hz, minor), 3.52 (s, 3H), 3.50 (s, minor), 3.41 (m, 1H), 3.30 (m, minor), 3.17 (m, minor), 3.13 (t, J=9.09 Hz, 1H), 3.04 (td, J=9.15, 7.04 Hz, 1H), 2.45 (m, 1H), 2.34 (m, minor), 2.30 (m, 1H), 2.24 (m, minor), 2.16 (ddd, J=13.59, 11.05, 4.44 Hz, 1H), 2.06-2.12 (om, 2H), 1.96 (s, IPAc, 3H), 1.84 (m, minor), 1.65-1.73 (om, 2H), 1.17 (d, J=6.22 Hz, IPAc, 6H), 0.94 (s, 9H), 0.89 (s, minor). ¹³C NMR (151 MHz, DMSO-d₆, 298K): d 177.5 (minor), 177.4, 170.8, 170.4 (minor), 169.8, 169.7 (IPAc), 169.4 (minor), 156.9, 156.3 (minor), 127.2 (1JCF=276.9 Hz, minor), 127.0 (1JCF=278.6 Hz), 119.5, 119.2 (minor), 66.9 (IPAc), 58.9, 58.5, 58.4 (minor), 51.4, 51.3 (minor), 46.7, 45.3 (minor), 41.2 (2JCF=28.14 Hz), 40.0 (minor), 39.6 (minor, ov), 39.1 (ov), 38.7 (2JCF=28.71 Hz, minor), 37.6, 37.3 (minor), 36.5, 35.1 (minor), 34.3, 34.1, 33.2 (minor), 30.4 (minor), 28.3 (minor), 28.1, 27.3 (minor), 26.8, 26.0, 25.9 (minor), 21.5 (IPAc), 21.0. ¹⁹F NMR (376 MHz, DMSO-d₆, 298K): d −70.71, −70.99 (minor), −71.12 (minor). om=overlapped multiplet, ov=overlapped with solvent, br=broad signal. HRMS: (ESI) m/z: [M+H]⁺ calculated for C₂₁H₃₁O₅N₅F₃ 490.2272 Da, found 490.2276 Da. PXRD was determined and is provided in FIG. 3.

### Example 8

### Preparation of Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate, anhydrous, Form 1 polymorph

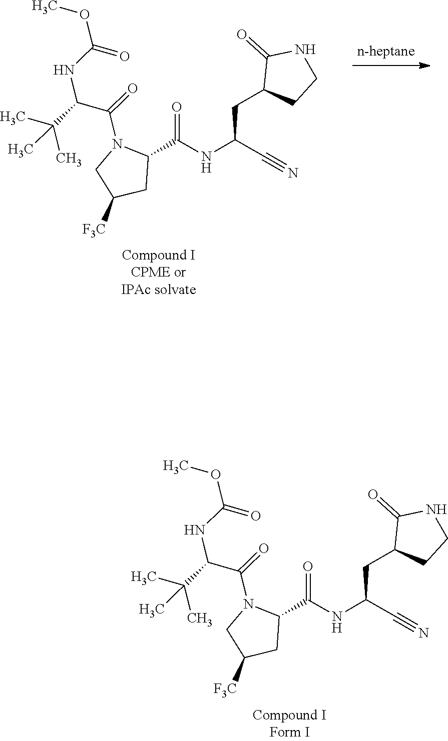 [SMILES: COC(=O)N[C@H](C(=O)N1C[C@H](C(F)(F)F)C[C@H]1C(=O)N[C@H](C#N)C[C@@H]1CCNC1=O)C(C)(C)C ; COC(=O)N[C@H](C(=O)N1C[C@H](C(F)(F)F)C[C@H]1C(=O)N[C@H](C#N)C[C@@H]1CCNC1=O)C(C)(C)C]

Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate (200 g, either the CPME Solvate or IPAc solvate form) is combined with heptane (2000 mL) and stirred at 20 °C. The mixture is then heated to 70 °C and stirred at 70 °C for at least 6 h. The mixture is then cooled to 50 °C and sampled to confirm conversion to the Form 1 polymorph. If the form is not Form 1, then the mixture is heated back to 70 °C for 6 h and the sampling is repeated. If it is Form 1, then the mixture is cooled to 20 °C over a period of 4 h, then held at 20 °C for at least 2 h. The slurry is filtered and washed with heptane (400 mL). The wet filter cake is then dried at 70 °C overnight, and Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate, Form 1 is isolated (96% yield)

### Product Characterization:

Note: The sample contains two minor rotamers due to the N1-C10 and N16-C17 amide bonds, making up approximately 6% and 10% of the sample, respectively. Where relevant, the less abundant rotamer signals are designated as “minor.”

¹H NMR (600 MHz, DMSO-d₆, 298K): δ 9.11 (d, J=6.97 Hz, minor), 9.05 (d, J=8.63 Hz, 1H), 7.75 (s, minor), 7.69 (s, 1H), 7.32 (d, J=8.66 Hz, 1H), 7.01 (d, J=9.16 Hz, minor), 6.90 (br, minor), 4.95 (ddd, J=11.23, 8.63, 4.86 Hz, 1H), 4.88 (q, J=7.53 Hz, minor), 4.80 (dd, J=8.16, 2.90 Hz, minor), 4.35 (t, J=7.53 Hz, 1H), 4.14 (d, J=8.78 Hz, 1H), 4.07 (br, minor), 4.04 (d, J=9.10 Hz, minor), 3.98-3.95 (m, 1H), 3.84 (br, minor), 3.65 (d, J=7.06 Hz, minor), 3.52 (s, 3H), 3.49 (s, minor), 3.41 (m, 1H), 3.31 (m, minor), 3.17 (m, minor), 3.13 (t, J=9.11 Hz, 1H), 3.03 (td, J=9.32, 7.17 Hz, 1H), 2.46 (m, 1H), 2.30 (m, 1H), 2.24 (m, minor), 2.16 (ddd, J=13.51, 11.25, 4.28 Hz, 1H), 2.12-2.05 (om, 2H), 1.84 (m, minor), 1.73-1.65 (om, 2H), 0.94 (s, 9H), 0.89 (s, minor). ¹³C NMR (151 MHz, DMSO-d₆, 298K): d 177.5 (minor), 177.4, 170.8, 170.4 (minor), 169.9 (minor), 169.7, 169.3 (minor), 156.9, 156.3 (minor), 155.5 (minor), 127.0 (1JCF=278.4 Hz), 119.5, 119.2 (minor), 59.4 (minor), 58.9, 58.8 (minor), 58.6 (minor), 58.5, 58.3 (minor), 51.6 (minor), 51.4, 51.3 (minor), 46.8 (minor), 46.7, 45.3 (minor), 41.1 (2JCF=28.0 Hz), 39.5 (minor, ov), 39.1, 39.0 (minor, ov), 38.6 (2JCF=27.8 Hz, minor), 37.6, 37.3 (minor), 36.5, 35.1 (minor), 34.3, 34.2 (minor), 34.1, 33.2 (minor), 30.3 (minor), 28.1, 27.3 (minor), 26.7, 26.0, 25.9 (minor). ¹⁹F NMR (376 MHz, DMSO-d₆, 298K): d −70.17, −70.45 (minor), −70.58 (minor). om=overlapped multiplet, ov=overlapped with solvent, br=broad signal. HRMS: (ESI) m/z: [M+H]⁺ calculated for C₂₁H₃₁O₅N₅F₃ 490.2272 Da, found 490.2271 Da.

### Example 9

### Preparation of Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate, anhydrous, Form 1 polymorph from Compound I, CPME Solvate

Compound I, CPME solvate (30.09 g, 51.03 mmol, 100 mass %) and Heptane (300 mL, 2047.9 mmol, 100 mass %) was added into a 1000 mL two-piece OptiMax reactor with overhead stirring at 350 rpm and baffle. The mixture was stirred at 20 °C and heated to 70 °C. The mixture was stirred at 70 °C for 12 hours then the mixture was cooled to 25 °C in 5 hours and stirred overnight. The resulting slurry was filtered and washed with Heptane (60 mL, 409.58 mmol, 100 mass %). The solids were dried at 50 °C under vacuum overnight to provide the anhydrous free form of N-(Methoxycarbonyl)-3-methyl-L-valyl-(4R)-N-{(1 S)-1-cyano-2-[(3S)-2-oxopyrrolidin-3-yl]ethyl}-4-(trifluoromethyl)-L-prolinamide, Form 1.

PXRD was determined for N-(Methoxycarbonyl)-3-methyl-L-valyl-(4R)-N-{(1S)-1-cyano-2-[(3S)-2-oxopyrrolidin-3-yl]ethyl}-4-(trifluoromethyl)-L-prolinamide, Form 1 and the PXRD pattern is provided in FIG. 1 and peaks are provided in the Table below.

TABLE
PXRD peak list with relative intensities for N-(Methoxycarbonyl)-
3-methyl-L-valyl-(4R)-N-{(1S)-1-cyano-2-[(3S)-2-oxopyrrolidin-
3-yl]ethyl}-4-(trifluoromethyl)-L-prolinamide, Form
1. PXRD peaks are in degrees 2Θ each ±0.2 Θ.

| Angle | Relative |
|---|---|
| (°2Θ) | Intensity (%) |
| 6.3 | 3 |
| 9.1 | 11 |
| 9.6 | 100 |
| 10.3 | 88 |
| 10.7 | 60 |
| 12.0 | 8 |
| 12.6 | 9 |
| 13.1 | 7 |
| 13.9 | 11 |
| 15.5 | 4 |
| 16.2 | 43 |
| 16.7 | 51 |
| 17.6 | 11 |
| 18.1 | 93 |
| 18.4 | 30 |
| 18.9 | 76 |
| 19.2 | 70 |
| 20.4 | 9 |
| 20.8 | 10 |
| 21.5 | 22 |
| 22.3 | 6 |
| 22.8 | 8 |
| 23.8 | 7 |
| 24.3 | 8 |
| 24.7 | 21 |
| 25.1 | 13 |
| 26.2 | 15 |
| 26.7 | 12 |
| 27.3 | 5 |
| 27.6 | 7 |
| 28.0 | 6 |
| 28.9 | 6 |
| 29.8 | 4 |
| 30.2 | 5 |
| 30.4 | 5 |
| 31.1 | 3 |
| 32.4 | 6 |
| 33.3 | 4 |
| 33.7 | 5 |
| 34.2 | 3 |

Single Crystal X-ray Diffraction (SXRD) was determined for N-(Methoxycarbonyl)-3-methyl-L-valyl-(4R)-N-{(1S)-1-cyano-2-[(3S)-2-oxopyrrolidin-3-yl]ethyl}-4-(trifluoromethyl)-L-prolinamide, Form 1 and the data is provided in the Table below.

TABLE
Crystal structure data of crystalline N-(Methoxycarbonyl)-
3-methyl-L-valyl-(4R)-N-{(1S)-1-cyano-2-[(3S)-2-oxopyrrolidin-
3-yl]ethyl}-4-(trifluoromethyl)-L-prolinamide, Form 1.

| | |
|---|---|
| Empirical formula | C₂₁H₃₀F₃N₅O₅ |
| Formula weight | 489.50 |
| Temperature | 298 |
| | K |
| Wavelength | 1.54178 |
| | Å |
| Crystal system | Orthorhombic |
| Space group | C222₁ |

| | | |
|---|---|---|
| Unit cell dimensions | a = 9.6317(4) Å | α = 90° |
| | b = 19.6368(8) Å | β = 90° |
| | c = 28.2776(11) Å | γ = 90° |

| | |
|---|---|
| Volume | 5348.3(4) |
| | Å³ |
| Z | 8 |
| Density (calculated) | 1.216 |
| | Mg/m³ |
| Goodness-of-fit on F² | 1.061 |
| Final R indices [I > 2sigma(I)] | R₁ = 0.0754, wR₂ = 0.2246 |
| R indices (all data) | R₁ = 0.0907, wR₂ = 0.2392 |

¹³C solid-state NMR was determined for N-(Methoxycarbonyl)-3-methyl-L-valyl-(4R)-N-{(1S)-1-cyano-2-[(3S)-2-oxopyrrolidin-3-yl]ethyl}-4-(trifluoromethyl)-L-prolinamide, Form 1.

TABLE
¹³C solid-state NMR peak list for N-(Methoxycarbonyl)-3-methyl-L-
valyl-(4R)-N-{(1S)-1-cyano-2-[(3S)-2-oxopyrrolidin-3-yl]ethyl}-4-
(trifluoromethyl)-L-prolinamide, Form 1. Each peak is ±0.2 ppm.

| ¹³C Chemical | Relative |
|---|---|
| Shift (ppm) | Intensity (%) |
| 180.6 | 29 |
| 173.4 | 28 |
| 170.6 | 28 |
| 156.8 | 27 |
| 126.7 | 5 |
| 119.7 | 9 |
| 59.1 | 30 |
| 58.3 | 24 |
| 50.8 | 34 |
| 47.8 | 12 |
| 43.5 | 21 |
| 41.6 | 22 |
| 38.7 | 33 |
| 37.6 | 27 |
| 36.7 | 34 |
| 29.5 | 13 |
| 28.0 | 20 |
| 26.5 | 100 |

¹⁹F solid-state NMR was determined for N-(Methoxycarbonyl)-3-methyl-L-valyl-(4R)-N-{(1S)-1-cyano-2-[(3S)-2-oxopyrrolidin-3-yl]ethyl}-4-(trifluoromethyl)-L-prolinamide, Form 1.

TABLE
¹⁹F solid-state NMR peak list for N-(Methoxycarbonyl)-3-methyl-
L-valyl-(4R)-N-{(1S)-1-cyano-2-[(3S)-2-oxopyrrolidin-
3-yl]ethyl}-4-(trifluoromethyl)-L-prolinamide, Form 1.

| ¹⁹F Chemical | Relative |
|---|---|
| Shift (ppm) | Intensity (%) |
| −70.7 | 100 |

TABLE
Examples of characterizing identifiers for N-(Methoxycarbonyl)-
3-methyl-L-valyl-(4R)-N-{(1S)-1-cyano-2-[(3S)-2-oxopyrrolidin-
3-yl]ethyl}-4-(trifluoromethyl)-L-prolinamide, Form 1,
using a single method or a combination of instrument methods.

| | ¹⁹F ssNMR | ¹³C ssNMR | PXRD |
|---|---|---|---|
| | (ppm) ± | (ppm) ± | (°2Θ) ± |
| Form 1 | 0.2 ppm | 0.2 ppm | 0.2 °2Θ |
| Single | | 50.8 | |
| Single | | 50.8, 58.3, 43.5 | |
| Combination | −70.7 | 50.8 | |
| Combination | −70.7 | 50.8, 58.3, 43.5 | |
| Combination | | 50.8 | at least one: 9.1, |
| | | | 9.6, 10.3, 16.2 |
| Combination | | 50.8, 58.3, 43.5 | at least one: 9.1, |
| | | | 9.6, 10.3, 16.2 |
| Combination | −70.7 | 50.8 | at least one: 9.1, |
| | | | 9.6, 10.3, 16.2 |
| Combination | −70.7 | 50.8, 58.3, 43.5 | at least one: 9.1, |
| | | | 9.6, 10.3, 16.2 |

1. A process for preparing a (2S,4R)-1-{(2S)-2-[(methoxycarbonyl)amino]-3,3-dimethylbutanoyl}-4-(trifluoromethyl)pyrrolidine-2-carboxylate M⁺ salt (Compound 6a) of formula: 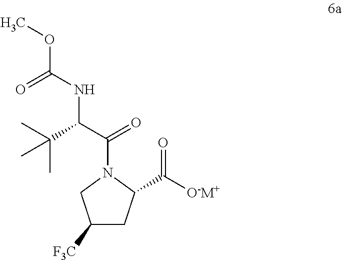
comprising the steps of
a) combining tert-butyl (2R,4R)-1-{(2S)-2-[(methoxycarbonyl)amino]-3,3-dimethylbutanoyl}-4-(trifluoromethyl)pyrrolidine-2-carboxylate (Compound 4) a solvent and water; and
b) adding a base comprising M⁺ to the mixture from step a) to generate intermediate tert-Butyl (2S,4R)-1-{(2S)-2-[(methoxycarbonyl)amino]-3,3-dimethylbutanoyl}-4-(trifluoromethyl)pyrrolidine-2-carboxylate, Compound 5, in situ which further reacts to provide the (2S,4R)-1-{(2S)-2-[(methoxycarbonyl)amino]-3,3-dimethylbutanoyl}-4-(trifluoromethyl)pyrrolidine-2-carboxylate M⁺ salt, Compound 6a 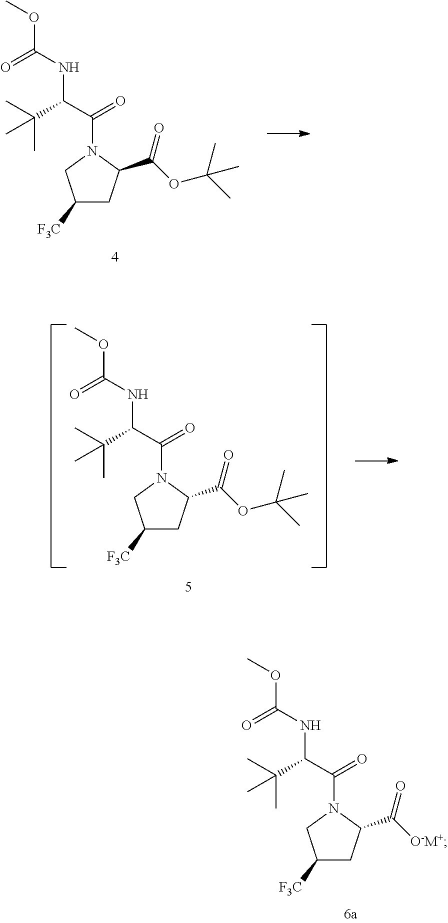
wherein M⁺ is selected from Li⁺, Na⁺ and K⁺.

2. The process of claim 1 wherein the base comprising M⁺ is selected from sodium tert-butoxide, sodium tert-amylate, potassium methoxide, potassium hydroxide, potassium tert-butoxide, potassium tert-amylate and potassium hexamethyldisilazide and the solvent is selected from the group consisting of methyl tert-butyl ether, tetrahydrofuran, isopropyl alcohol, tert-amyl alcohol, acetonitrile, tert-butanol and 2-methyl tetrahydrofuran.

3. The process of claim 1 for preparing potassium (2S,4R)-1-{(2S)-2-[(methoxycarbonyl)amino]-3,3-dimethylbutanoyl}-4-(trifluoromethyl)pyrrolidine-2-carboxylate, Compound 6 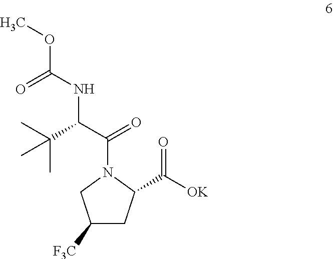
a) combining tert-Butyl (2R,4R)-1-{(2S)-2-[(methoxycarbonyl)amino]-3,3-dimethylbutanoyl}-4-(trifluoromethyl)pyrrolidine-2-carboxylate, Compound 4, a solvent and water; and
b) adding a base comprising K⁺ to the mixture from step a) to generate intermediate tert-Butyl (2S,4R)-1-{(2S)-2-[(methoxycarbonyl)amino]-3,3-dimethylbutanoyl}-4-(trifluoromethyl)pyrrolidine-2-carboxylate, Compound 5, in situ which further reacts to provide potassium (2S,4R)-1-{(2S)-2-[(methoxycarbonyl)amino]-3,3-dimethylbutanoyl}-4-(trifluoromethyl)pyrrolidine-2-carboxylate, Compound 6

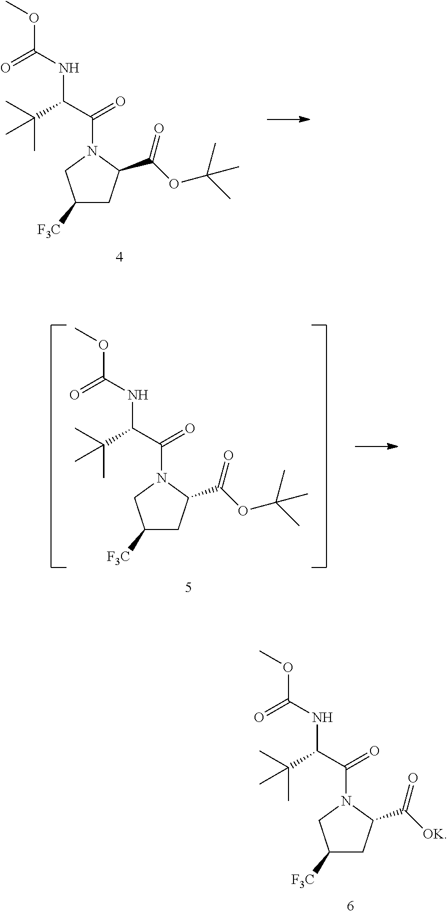

4. The process of claim 3 wherein the base comprising K⁺ is selected from the group consisting of potassium tert-butoxide, potassium tert-amylate, potassium methoxide, potassium hydroxide and potassium hexamethyldisilazide and the solvent is selected from the group consisting of methyl tert-butyl ether, tetrahydrofuran, isopropyl alcohol, tert-amyl alcohol, acetonitrile, tert-butanol and 2-methyl tetrahydrofuran.

5. The process of claim 4 wherein the base comprising K⁺ is potassium tert-butoxide.

6. The process of claim 5 wherein in step a) 1.0 equivalent of tert-Butyl (2R,4R)-1-{(2S)-2-[(methoxycarbonyl)amino]-3,3-dimethylbutanoyl}-4-(trifluoromethyl)pyrrolidine-2-carboxylate, Compound 4 is combined with 2.5 volumes of the solvent methyl tert-butyl ether (MTBE) and 1.2 equivalents of water at ambient temperature and in step b) 1.2 equivalents of potassium tert-butoxide (20 weight % in THF) is added at ambient temperature.

7. The process of claim 6 further comprising the steps c)-g) wherein
step c) stirring the reaction mixture obtained from step b) at ambient temperature for a period of at least 12 hours;
step d) addition of 0.5 volumes of methanol with stirring after step c) to provide a reaction mixture slurry;
step e) filtering the reaction mixture slurry obtained from step d) to isolate a solid comprising potassium (2S,4R)-1-{(2S)-2-[(methoxycarbonyl)amino]-3,3-dimethylbutanoyl}-4-(trifluoromethyl)pyrrolidine-2-carboxylate, Compound 6;
step f) washing the solid comprising potassium (2S,4R)-1-{(2S)-2-[(methoxycarbonyl)amino]-3,3-dimethylbutanoyl}-4-(trifluoromethyl)pyrrolidine-2-carboxylate, Compound 6 obtained from step e) with a mixture of MTBE (1.7 volumes) and methanol (0.3 volumes); and
step g) drying the solid obtained in step f) comprising potassium (2S,4R)-1-{(2S)-2-[(methoxycarbonyl)amino]-3,3-dimethylbutanoyl}-4-(trifluoromethyl) pyrrolidine-2-carboxylate, Compound 6.

8. The process of claim 7 wherein the isolated solid comprises greater than or equal to 95% by weight of crystalline potassium (2S,4R)-1-{(2S)-2-[(methoxycarbonyl)amino]-3,3-dimethylbutanoyl}-4-(trifluoromethyl)pyrrolidine-2-carboxylate, Compound 6.

9. The process of claim 8 wherein the isolated solid comprises less than 5% by weight of potassium (2R,4R)-1-{(2S)-2-[(methoxycarbonyl)amino]-3,3-dimethyl butanoyl}-4-(trifluoromethyl)pyrrolidine-2-carboxylate.

10. A process for preparing a solvate of Methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate (Compound I) 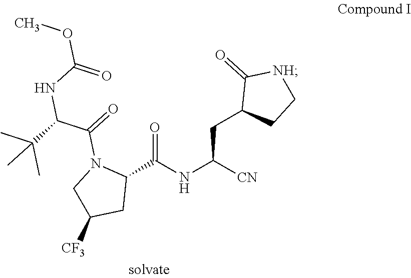 [SMILES: COC(=O)N[C@H](C(=O)N1C[C@H](C(F)(F)F)C[C@H]1C(=O)N[C@H](C#N)C[C@@H]1CCNC1=O)C(C)(C)C]
comprising the steps of
h) reacting (2S,4R)-1-{(2S)-2-[(methoxycarbonyl)amino]-3,3-dimethylbutanoyl}-4-(trifluoromethyl)pyrrolidine-2-carboxylic acid, Compound 6′, or a pharmaceutically acceptable salt thereof with (2S)-2-Amino-3-[(3S)-2-oxopyrrolidin-3-yl]propanamide, Compound 7′, or a pharmaceutically acceptable salt thereof in the presence of a peptide coupling reagent, a base and a solvent 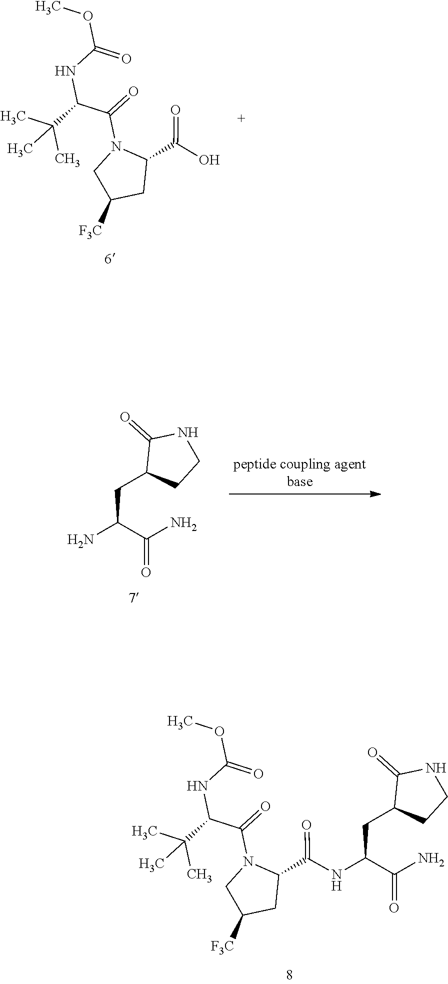 [SMILES: COC(=O)N[C@H](C(=O)N1C[C@H](C(F)(F)F)C[C@H]1C(=O)O)C(C)(C)C ; NC(=O)[C@@H](N)C[C@@H]1CCNC1=O ; COC(=O)N[C@H](C(=O)N1C[C@H](C(F)(F)F)C[C@H]1C(=O)N[C@@H](C[C@@H]1CCNC1=O)C(N)=O)C(C)(C)C]
to provide methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate, Compound 8; and
i) reacting Compound 8 with a dehydrating reagent 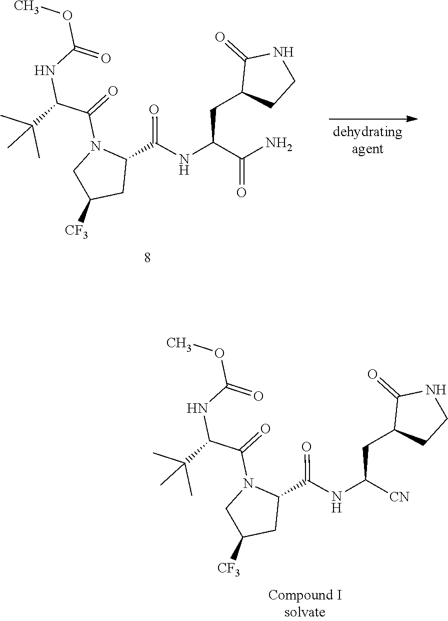 [SMILES: COC(=O)N[C@H](C(=O)N1C[C@H](C(F)(F)F)C[C@H]1C(=O)N[C@@H](C[C@@H]1CCNC1=O)C(N)=O)C(C)(C)C ; COC(=O)N[C@H](C(=O)N1C[C@H](C(F)(F)F)C[C@H]1C(=O)N[C@H](C#N)C[C@@H]1CCNC1=O)C(C)(C)C]
to provide a solvate of methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate, Compound I solvate.

11. The process of claim 10 wherein in step h) the peptide coupling reagent is selected from 2-chloro-1-methylpyridinium p-toluenesulfonate, a combination of 2-hydroxy pyridine N-oxide and 1-ethyl-3-(3-dimethylaminopropyl)carbodiimide and a combination of 2-hydroxypyridine N-oxide and 1-ethyl-3-(3-dimethylaminopropyl) carbodiimide hydrochloride and the base is triethylamine, N-methyl morpholine or N,N-diisopropylethylamine.

12. The process of claim 11 wherein the solvent in step h) is methyl ethyl ketone or isopropyl acetate.

13. The process of claim 12 wherein in step i) the dehydrating agent is selected from trifluoroacetic anhydride, propane phosphonic acid anhydride, triphenylphosphite, diethyl chlorophosphate, ethyl dichlorophosphate, phosphorus trichloride and tris-(dimethylamino)phosphine in the presence of a base selected from N-methylimidazole, N-methyl morpholine, diethylamine, triethylamine and 1,8-Diazabicyclo(5.4.0)undec-7-ene.

14. The process of claim 13 wherein in step i) the dehydrating agent is trifluoroacetic anhydride or propane phosphonic acid anhydride.

15. The process of claim 14 wherein isopropyl acetate is used as the solvent in steps h) and i) and the reaction mixture from step i) is concentrated and to it is added heptane to provide methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate isopropyl acetate solvate, Compound I IPAc solvate.

16. The process of claim 13 wherein in step h) the solvent is methyl ethyl ketone, the peptide coupling agent is a combination of 2-hydroxypyridine N-oxide and 1-ethyl-3-(3-dimethylaminopropyl)carbodiimide and the base is triethylamine.

17. The process of claim 16 wherein after the coupling reaction is complete the reaction mixture from step h) the solvent is exchanged to isopropyl acetate by addition of isopropyl acetate and distillation and in step i) the dehydrating agent is trifluoroacetic anhydride and the base is N-methyl morpholine.

18. The process of claim 17 wherein the reaction mixture from step i) is quenched with aqueous ammonium hydroxide, the layers are separated and the isopropyl acetate layer is solvent exchanged into cyclopentyl methyl ether by addition of cyclopentyl methyl ether and distillation.

19. The process of claim 18 wherein the cyclopentyl methyl ether mixture from step i) is cooled to 10 °C and stirred at 10 °C for one hour or longer and methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate cyclopentyl methyl ether solvate, Compound I CPME solvate is isolated.

20.-22. (canceled)

23. A process for preparing methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate ethyl acetate solvate (Compound I, ethyl acetate solvate) 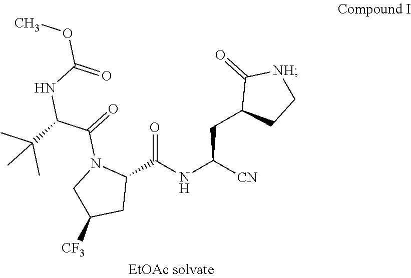 [SMILES: COC(=O)N[C@H](C(=O)N1C[C@H](C(F)(F)F)C[C@H]1C(=O)N[C@H](C#N)C[C@@H]1CCNC1=O)C(C)(C)C]
comprising the steps of
h′) reacting potassium (2S,4R)-1-{(2S)-2-[(methoxycarbonyl)amino]-3,3-dimethylbutanoyl}-4-(trifluoromethyl)pyrrolidine-2-carboxylate, Compound 6 with (2S)-2-amino-3-[(3S)-2-oxopyrrolidin-3-yl]propanamide hydrochloride (1:1), Compound 7 in the presence of 2-chloro-1-methylpyridinium p-toluenesulfonate and a base 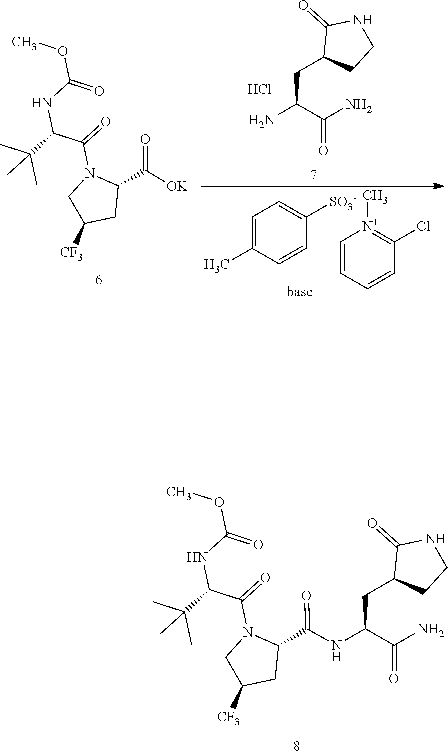
to provide methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate, Compound 8; and
i′) reacting Compound 8 with trifluoroacetic anhydride in the presence of N-methylimidazole in ethyl acetate 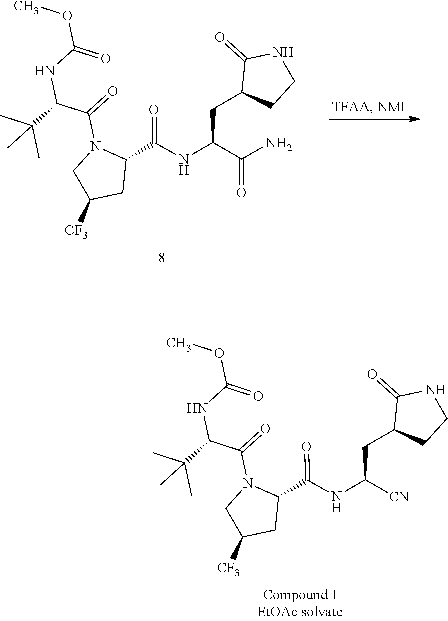 [SMILES: COC(=O)N[C@H](C(=O)N1C[C@H](C(F)(F)F)C[C@H]1C(=O)N[C@@H](C[C@@H]1CCNC1=O)C(N)=O)C(C)(C)C ; COC(=O)N[C@H](C(=O)N1C[C@H](C(F)(F)F)C[C@H]1C(=O)N[C@H](C#N)C[C@@H]1CCNC1=O)C(C)(C)C]
to provide methyl {(2S)-1-[(2S,4R)-2-({(1S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate ethyl acetate solvate, Compound I EtOAc solvate.

24. The process of claim 23 wherein step h′) is carried out in methyl ethyl ketone and water.

25. The process of claim 24 wherein 1.0 equivalent of potassium (2S,4R)-1-{(2S)-2-[(methoxycarbonyl)amino]-3,3-dimethylbutanoyl}-4-(trifluoromethyl)pyrrolidine-2-carboxylate, Compound 6 is reacted with 1.15 equivalents of (2S)-2-Amino-3-[(3S)-2-oxopyrrolidin-3-yl]propanamide hydrochloride (1:1), Compound 7 in the presence of 1.2 equivalents of 2-chloro-1-methylpyridinium p-toluenesulfonate and the base is 3.0 equivalents of N-methyl morpholine.

26. The process of claim 25 where the reaction mixture in step h′) is stirred for 4 hours at 20 °C; the crude reaction mixture from step h′) is solvent exchanged into ethyl acetate by vacuum distillation until the water content is less than 0.2% by weight to provide a slurry of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate, Compound 8 in ethyl acetate; and in step i′) the slurry of Methyl {(2S)-1-[(2S,4R)-2-({(2S)-1-amino-1-oxo-3-[(3S)-2-oxopyrrolidin-3-yl]propan-2-yl}carbamoyl)-4-(trifluoromethyl)pyrrolidin-1-yl]-3,3-dimethyl-1-oxobutan-2-yl}carbamate, Compound 8 in ethyl acetate is cooled to 0 °C and 5.0 equivalents of N-methylimidazole is added followed by addition of 2.5 equivalents of trifluoroacetic anhydride over 30 minutes then the reaction mixture is stirred at 0 °C for 1 hour.

27. The process of claim 26 further comprising step j) quenching the reaction mixture from step i′) with an aqueous mixture of citric acid monohydrate and sodium chloride and the resulting layers are separated and the aqueous layer is back extracted with ethyl acetate and the combined organic ethyl acetate layers are concentrated in vacuo to approximately half the original volume, heptane is added then the methyl {(2S)-1-[(2S,4R)-2-({(1 S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate ethyl acetate solvate is isolated by filtration.

28. The process of claim 27 further comprising the step of combining the methyl {(2S)-1-[(2S,4R)-2-({(1S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate ethyl acetate solvate with heptane and the mixture is stirred at 60 °C to 80 °C for a period of 6 hours to 24 hours to provide methyl {(2S)-1-[(2S,4R)-2-({(1 S)-1-cyano-2-[(3S)-2-oxopyrrolidin-3-yl]ethyl}carbamoyl)-4-(trifluoromethyl)pyrrolidine-1-yl]-3,3-dimethyl-1-oxobutan-2-yl}carbamate, Compound I Form 1 wherein the Compound I Form 1 is characterized by ¹³C solid state NMR peaks at 50.8 ppm, 58.3 ppm and 43.5 ppm wherein each peak is ±0.2 ppm, a ¹⁹F solid state NMR peak at −70.7 ppm±0.2 ppm and one to four powder X-ray diffraction peaks (Cu Kα radiation) selected from the group consisting of peaks at 9.1, 9.6, 10.3 and 16.2 degrees 2θ wherein each peak is ±0.2 degrees 2θ.

29. The process of claim 28 wherein the mixture is stirred at about 70 °C for 6 hours to 12 hours then the mixture is cooled to 20 °C over 4 hours and held at 20 °C for 2 hours then the methyl {(2S)-1-[(2S,4R)-2-({(1 S)-1-cyano-2-[(3S)-2-oxopyrrolidin-3-yl]ethyl}carbamoyl)-4-(trifluoromethyl) pyrrolidine-1-yl]-3,3-dimethyl-1-oxobutan-2-yl}carbamate, Compound I Form 1 is isolated.

30.-32. (canceled)

33. The compound (2S,4R)-1-{(2S)-2-[(methoxycarbonyl)amino]-3,3-dimethylbutanoyl}-4-(trifluoromethyl)pyrrolidine-2-carboxylic acid; or a salt thereof.

34. The compound of claim 33 wherein the salt is selected from lithium, sodium and potassium.

35. The compound of claim 34 which is potassium (2S,4R)-1-{(2S)-2-[(methoxycarbonyl) amino]-3,3-dimethylbutanoyl}-4-(trifluoromethyl)pyrrolidine-2-carboxylate.

36.-38. (canceled)
